(19) United States
(12) Patent Application Publication
(10) Pub. No.: US 2022/0409281 A1
(43) Pub. Date: Dec. 29, 2022

(54) SYSTEMS AND METHODS FOR VENTRICLE PROCEDURES

(71) Applicant: The Brigham and Women's Hospital, Inc., Boston, MA (US)

(72) Inventors: William B. Gormley, Boston, MA (US); Junichi Tokuda, Boston, MA (US); Faith C. Robertson, Boston, MA (US); Paola Calvachi Prieto, Boston, MA (US); Franklin King, Boston, MA (US); Steve Pieper, Boston, MA (US)

(21) Appl. No.: 17/778,722

(22) PCT Filed: Nov. 23, 2020
(86) PCT No.: PCT/US2020/061840

§ 371 Date: May 20, 2022

Related U.S. Application Data
(60) Provisional application No. 62/939,310, filed on Nov. 22, 2019.

(51) Int. Cl. A61B 34/10; A61B 34/00; A61B 90/00

(52) U.S. Cl. CPC A61B 34/10; A61B 34/25; A61B 90/36; A61B 2034/107; A61B 2034/105; A61B 2090/103

(57) ABSTRACT

A computing device having a first processor configured to receive three-dimensional imaging data acquired by an imaging system, the three-dimensional imaging data being from ahead of a subject. The first processor can be configured to determine vascular structures from the three-dimensional imaging data, generate a three-dimensional model of the head of the subject from the three-dimensional imaging data, the three-dimensional model of the head including a three-dimensional model of the vascular structures and a three-dimensional model of a portion of a frontal horn of the subject, determine an entry point on the three-dimensional model of the head of the subject, determine a plurality of trajectories, each trajectory intersecting the three-dimensional model of the portion of the frontal horn and does not intersect the three-dimensional model of the vascular structures, and each trajectory is linear, and select a final trajectory from the plurality of trajectories.

### CROSS-REFERENCE TO RELATED APPLICATIONS

This application claims priority to U.S. Patent Application No. 62/939,310 filed Nov. 22, 2019, and entitled, “Systems and Methods for External Ventriculostomy Guidance,” which is hereby incorporated by reference in its entirety.

### STATEMENT REGARDING FEDERALLY SPONSORED RESEARCH

N/A

### BACKGROUND

Typically, the need to access the ventricular system of the brain is extremely common during the treatment of hydrocephalus. For example, the immediate presentation of hydrocephalus related symptoms is often accompanied by, or caused by, other disease states. In some instances, a brain hemorrhage within the ventricular system can cause obstruction of flow of the Cerebral Spinal Fluid (“CSF”), a key symptom of hydrocephalus. In other instances, obstruction of CSF can be caused by a tumor or mass, while in still further instances, obstruction of CSF can be caused by cerebral trauma or swelling (e.g., from an infection).

Conventionally, a ventriculostomy (a neurosurgical procedure for the treatment of acute hydrocephalus) is performed with an unguided, freehand, technique to place an External Ventricular Device (“EVD”) within the ventricle of the subject's brain. This manual placement of an EVD (e.g., a catheter) without any technological assistance, is common in the hospital setting at least because of the time-sensitive nature of the oncoming symptoms. In other words, an EVD is typically placed manually at the bedside because the subject is most likely suffering from related symptoms (e.g., a brain hemorrhage), which is likely to cause the CSF obstruction to become exacerbated in a relatively short period of time.

Unfortunately, the manual placement of an EVD is often errant and requires multiple attempts. It is estimated that accurate placement is accomplished between 40-60 percent of the time. Thus, although this procedure is performed frequently and presumed to be simple, it is considered the neurosurgical procedure with the highest complication rate, which can include hemorrhage, and EVD misplacement.

Considering that over 200,000 new cases of hydrocephalus sprout up each year in the United States alone, it would be desirable to have improved systems and methods for EVD guidance, including improved systems and methods for ventricle procedures.

### SUMMARY OF THE DISCLOSURE

Some non-limiting examples of the disclosure provide improved surgical systems for treating disease states of a ventricle of a brain of a subject. For example, some non-limiting examples can determine a desired trajectory for a medical instrument to travel through the brain of a subject. In some cases, once the ideal trajectory has been established for entry into the ventricular system, and the medical device (e.g., an expandable ventricular catheter) has been deployed, then the medical device can expand to create a conduit for which further surgical interventions can be conducted through. This can provide reduced surgical dissection and thus lower surgical risk. In this sense, the placement of a desiredly placed ventricular catheter (or along the ideal trajectory) serves for the purposes inherent of all such catheters, the draining of CSF, but additionally serves for subsequent surgery to be carried out through that surgical corridor to provide a minimally invasive corridor for further and future surgical interventions.

Some non-limiting examples of the disclosure provide improved surgical systems for deployment of a medical instrument along a desired trajectory. For example, once the ideal surgical trajectory has been determined (e.g., from a plurality of options), deployment systems that can include robotics can assist with bedside placement of the medical instrument (e.g., the ventricular catheter) along this ideal trajectory. Importantly, this can be accomplished for a patient that is otherwise not sedated, not immobilized, in other words, mobile in a setting where extensive optical tracking and surgical assistance is not available such as an operating room setting.

In some non-limiting examples, once this trajectory has been determined, the medical instrument can be deployed. For example, computer assisted, robotic guidance of the medical instrument to a target location can cause the medical device to follow a complex path that includes a linear component (e.g., a three-dimensional line that is the ideal trajectory) and a non-linear component that extends from an end of the three-dimensional line. Once advanced along the complex path and to the desired location within the ventricle, the medical device can be expanded to create an internal volume that defines a “structured surgical corridor” along the three-dimensional line and beyond the tip of the ventricular catheter to the desired site. This “structured surgical corridor” can allow for any site of the brain to be accessible though the spinal fluid containing spaces of the brain including the ventricle and sub arachnoid space.

Some non-limiting examples of the disclosure provide a system and method for guiding a medical instrument. The system includes a computing device having a first processor configured to receive three-dimensional imaging data acquired by an imaging system, the three-dimensional imaging data being from a head of a subject. The first processor can be configured to determine vascular structures from the three-dimensional imaging data, and generate a three-dimensional model of the head of the subject from the three-dimensional imaging data. The three-dimensional model of the head can include a three-dimensional model of the vascular structures and a three-dimensional model of a portion of a frontal horn of the subject. The first processor can be configured to determine an entry point on the three-dimensional model of the head of the subject, and determine a plurality of trajectories for the medical instrument. Each trajectory within the plurality of trajectories can intersect the three-dimensional model of the portion of the frontal horn and does not intersect the three-dimensional model of the vascular structure. Each trajectory within the plurality of trajectories can be linear. The first processor can be configured to select a final trajectory for the medical instrument from the plurality of trajectories.

Some non-limiting examples of the disclosure provide a trajectory determination system for guiding a medical instrument. The system can include a computing device, the computing device having a first processor. The first processor can be configured to receive three-dimensional imaging data acquired by an imaging system, the three-dimensional imaging data being from a head of a subject, determine vascular structures from the three-dimensional imaging data, and generate a three-dimensional model of the head of the subject from the three-dimensional imaging data, the three-dimensional model of the head including a three-dimensional model of the vascular structures and a three-dimensional model of a portion of a frontal horn of the subject. The first processor can be configured to determine an entry point on the three-dimensional model of the head of the subject and determine a plurality of trajectories for the medical instrument, wherein each trajectory within the plurality of trajectories intersects the three-dimensional model of the portion of the frontal horn and does not intersect the three-dimensional model of the vascular structures, and wherein each trajectory within the plurality of trajectories is linear. The first processor can be configured to select a final trajectory for the medical instrument from the plurality of trajectories.

In some non-limiting examples, each trajectory is a three-dimensional linear trajectory.

In some non-limiting examples, the three-dimensional imaging data includes a plurality of images of the head of the subject, and a machine learning model identifies the vascular structures from the plurality of images. In some non-limiting examples, the first processor is configured to generate the three-dimensional model of the vascular structures from the identification of the vascular structures by the machine learning model.

In some non-limiting examples, the first processor can be further configured to refine the plurality of trajectories to determine a final trajectory being a single trajectory within the plurality of trajectories.

In some non-limiting examples, the first processor can be further configured to determine for each given trajectory, the closest distance between the given trajectory and the three-dimensional model of the vascular structures, and determine the final trajectory by identifying the trajectory with the smallest closest distance between the given trajectory and the three-dimensional model of the vascular structures.

In some non-limiting examples, the first processor can be further configured to generate a three-dimensional model of a mask to be worn by the subject, based on the final trajectory.

In some non-limiting examples, the mask can include an anatomical feature engaging region, and a bore directed through the mask, the bore aligning with the final trajectory when the mask interfaces with a head of the subject.

In some non-limiting examples, a central point of the bore aligns with the entry point of the trajectory, and the mask further includes an extruded cylinder surrounding the bore, the extruded cylinder aligning with the final trajectory.

In some non-limiting examples, the first processor is in communication with a three-dimensional printer. In some non-limiting examples, the first processor can be further configured to transmit the three-dimensional model of the mask to the three-dimensional printer, and the three-dimensional printer can be configured to print the mask.

In some non-limiting examples, the-dimensional printer is configured to print the mask within a time period, the time period being an amount of time necessary to prepare the subject for a surgical procedure.

In some non-limiting examples, the time period is under 60 minutes.

In some non-limiting examples, the mask is dimensioned, such that the three-dimensional printer is configured to print the mask within the time period. In some non-limiting examples, these dimensions can include a thickness of the mask, and a surface area of the mask that engages with the subject's head.

In some non-limiting examples, the trajectory determination system can include a cranial screw having a hole therethrough, the cranial screw being dimensioned to be received within the bore of the mask.

In some non-limiting examples, the trajectory determination system can include an external ventricular device including a catheter, the catheter being configured to be received through the hole of the cranial screw.

In some non-limiting examples, the trajectory determination system can include a surgical system including a guidewire, the guidewire being configured to be received through the catheter of the external ventricular device.

In some non-limiting examples, the trajectory determination system can include a guidance system. The guidance system can include a second processor in communication with the first processor, a first camera and a second camera, the first camera and the second camera being in communication with the second processor. The guidance system can include a projector in communication with the second processor, a mounting unit coupled to the first camera and the second camera. The second processor can be configured to receive the final trajectory from the computing device, cause the first camera to acquire a first image of the head of the subject, and the second camera to acquire a second image of the head of the subject, construct a three-dimensional surface map of the head of the subject from the first image and the second image, and register the three-dimensional surface map relative to the three-dimensional model of the head of the subject to track the guidance system relative to the subject.

In some non-limiting examples, the projector is coupled to the mounting unit. In some non-limiting examples, the second processor can be further configured to cause the first camera to acquire a third image of the head of the subject and the second camera to acquire a fourth image of the head of the subject when the subject has been draped and an optical marker has been placed on the head of the subject, the third image and the fourth image containing the optical marker, and track the position of the guidance system relative to the subject based on the location of the optical marker within the third and fourth images.

In some non-limiting examples, the second processor can be further configured to cause the projector to emit an illumination pattern towards the subject's head, the illumination pattern aligning with the final trajectory.

In some non-limiting examples, the processor can be further configured to adjust an angle the illumination pattern is emitted from the guidance system, such that the illumination pattern remains aligned with the final trajectory.

In some non-limiting examples, the trajectory determination system can include an augmented reality headset in communication with the second processor of the guidance system, the augmented reality headset can be configured to project a 3D image scene to a user wearing the augmented reality headset, the 3D image scene including the three-dimensional model of the head of the subject and the final trajectory displayed relative to the three-dimensional model of the head. The augmented reality headset can be configured to track the position of the augmented reality headset relative to the guidance system, and adjust the projection of the 3D image scene to the user, based on tracked position of the augmented reality headset relative to the guidance system.

In some non-limiting examples, the trajectory determination system can include a pivotable screw, the pivotable screw having a main body and a pivotable member received within the main body, the pivotable member being configured to adjust an orientation of the pivotable member relative to the main body. In some non-limiting examples, the pivotable screw is configured to receive a catheter of an external ventricular device.

In some non-limiting examples, the pivotable screw includes a locking member, the locking member being configured to fix the orientation of the pivotable member relative to the main body.

In some non-limiting examples, the first processor of the computing device can be further configured to display the final trajectory and the three-dimensional model of the head of the subject on a display in communication with the computing device.

Some non-limiting examples of the disclosure provide a surgical system. The surgical system can include a cranial screw, and a sheath system. The sheath system can include a body having a first conduit and a second conduit extending therethrough, the first conduit and the second conduit being in fluid communication, and an adapter coupled to the body and the cranial screw. The surgical system can include a catheter being configured to extend through the first conduit, into the adapter, and through the cranial screw. In some non-limiting examples, the catheter is configured to receive a surgical instrument. In some non-limiting examples, the adapter is a telescopic adapter. In some non-limiting examples, one end of the adapter is removably coupled to the body and an opposite end of the adapter is removably coupled to the cranial screw.

In some non-limiting examples, the surgical system can include at least one of a camera, an endoscope, a computed tomography system, a magnetic resonance imaging system, or an ultrasonography system, to guide the surgical instrument through the catheter and into the subject.

In some non-limiting examples, when the catheter is implanted in a subject, cerebrospinal fluid of the subject travels through the catheter and into the second conduit.

In some non-limiting examples, when the catheter is implanted in a subject and cerebrospinal fluid of the subject travels through the catheter and into the second conduit, the catheter is configured to receive the surgical instrument to perform an intraventricular surgical procedure.

Some non-limiting examples of the disclosure include a robotic surgical system. The robotic surgical system can include a housing defining an axial axis, a shaft rotatable about the axial axis, a first motor configured to rotate the shaft, a surgical scaffold having an end including a wire coupled thereto and extending along a longitudinal extent of the surgical scaffold, the end of the surgical scaffold defining a tip of the surgical scaffold, a second motor configured to provide tension to the wire of the surgical scaffold, and a computing device in communication with the first motor and the second motor. The computing device can be configured to receive a final trajectory for the surgical scaffold, the final trajectory being a three-dimensional (3D) line, cause the second motor to pull the wire of the surgical scaffold thereby deflecting the tip of the surgical scaffold towards the housing, and cause the first motor to rotate the shaft about the axial axis thereby rotating the surgical scaffold about the axial axis. In some non-limiting examples, activation of the first and second motors aligns the tip of the surgical scaffold along the 3D line.

In some non-limiting examples, the computing device can be configured to cause the second motor to pull the wire of the surgical scaffold to deflect the tip until a two-dimensional (2D) orientation of the tip of the surgical scaffold aligns with the corresponding dimensions of the 3D line, and cause the first motor to rotate the shaft about the axial axis until the 2D orientation of the tip of the surgical scaffold aligns with the 3D line.

In some non-limiting examples, the first motor forms part of a linear actuator, the linear actuator can include a lead screw angled relative to the axial axis, and a nut threadingly engaged with the lead screw. In some non-limiting examples, an end of the wire is coupled to the nut so that rotation of the first motor translates the nut to thereby increase or decrease tension on the wire.

In some non-limiting examples, the robotic surgical system can include an advancer configured to translationally move the surgical scaffold along the axial axis. In some non-limiting examples, the computing device is in communication with the advancer to cause the advancer to advance or retreat the surgical scaffold along the axial axis.

In some non-limiting examples, the advancer is a linear actuator. In some non-limiting examples, a component of the surgical scaffold is coupled to the linear actuator so that rotation of the linear actuator translationally moves the component of the surgical scaffold thereby translationally moving the surgical scaffold.

In some non-limiting examples, the component of the surgical scaffold includes a plunger assembly that is configured to be removably coupled to a surgical scaffold.

In some non-limiting examples, the computing device can be configured to after aligning the tip of the surgical scaffold along the 3D line, causing the advancer to advance the surgical scaffold away from the housing and along the 3D line.

In some non-limiting examples, the robotic surgical system can include a robot arm having six degrees of freedom, and an end effector. In some non-limiting examples, the housing is the end effector of the robot arm.

In some non-limiting examples, the computing device can be configured to receive 3D coordinates of an entry point on the subject, and cause the robot arm to move to align the shaft with the entry point.

In some non-limiting examples, the computing device can be configured to move the robot arm to align the shaft with the 3D line.

Some non-limiting examples of the disclosure provide a surgical system for a ventricle of a brain of a subject. The surgical system can include an expandable catheter that is a ventricular catheter, the expandable catheter is configured to radially expand from a compressed state to an expanded state, and a surgical scaffold configured to be coaxially received within the expandable catheter when the surgical scaffold is in a compressed state, the surgical scaffold being configured to radially expand from the compressed state to an expanded state.

In some non-limiting examples, the surgical system can include an instrument that is configured to be received through the surgical scaffold and the expandable catheter.

In some non-limiting examples, the instrument is at least one of an aspirator, a laser, a tissue vaporizer, a coagulation device, a hemostatic device, or a cutting device.

Some non-limiting examples of the disclosure provide a computer-implemented method for conducting a neurosurgical procedure. The method can include receiving, using one or more computing devices, a final trajectory that is a 3D line, projecting, using the one or more computing device, an illumination pattern that extends along the 3D line, aligning, using the one or more computing devices, a portion of an medical instrument so that the portion of the medical instrument aligns with the 3D line, and advancing, using the one or more computing devices, the medical instrument to extend farther along the 3D line and into a ventricle of a subject.

### BRIEF DESCRIPTION OF THE DRAWINGS

FIG. 1A is an example of an x-ray computed tomography imaging system.

FIG. 1B is a schematic illustration showing specifics of the x-ray computed tomography imaging system of FIG. 1A.

FIG. 2 is an example of a generalized system and process architecture for improved EVD placement.

FIG. 3 is a schematic illustration of an example of a determine trajectory process within the improved EVD placement system and process architecture of FIG. 2.

FIG. 4 is an example of a 3D model of the head of the subject, showing the determined Naasion and Kocher points.

FIG. 5 is an axial slice of an image of the subject's head, with boundaries defined to generate the 3D model of the frontal horn.

FIG. 6 is an example of a display of the system described in FIG. 2 running a machine learning model that identifies and segments cortical vascular structures.

FIG. 7 is a snapshot of the display of the system described in FIG. 2, illustrating the plurality of trajectories.

FIG. 8 is a schematic illustration of another guidance system, which is a specific implementation of the guidance system described in FIG. 2.

FIG. 9 is a front perspective view of a digitally rendered custom mask interfacing with the 3D model of FIG. 7.

FIG. 10 is a rear perspective view of the digitally rendered custom mask of FIG. 9.

FIG. 11 is a front perspective view of the custom mask of FIG. 9, printed from a 3D printer.

FIG. 12 is another front perspective view of the custom mask of FIG. 11.

FIG. 13 is an example of a mask attachment, which interfaces with the custom mask of FIG. 11.

FIG. 14 is a top perspective view of the mask attachment of FIG. 13 installed with the custom mask of FIG. 11.

FIG. 15 is a front perspective view of the mask attachment of FIG. 13 installed with the custom mask of FIG. 11.

FIG. 16 is an illustration of another custom mask, which is an alternative non-limiting example of the custom mask of FIG. 11.

FIG. 17 is an illustration of the custom mask of FIG. 16, with an installed port.

FIG. 18 is a front perspective view of an example of a cranial screw.

FIG. 19 is a schematic illustration of a sheath system interfacing with the cranial screw of FIG. 18.

FIG. 20 is a front view of a telescopic adapter of the sheath system of FIG. 19, in a fully extended configuration.

FIG. 21 is a cross-sectional view of the telescopic adapter of FIG. 20.

FIG. 22 is a front view of the telescopic adapter of FIG. 20, in a fully retracted configuration.

FIG. 23 is an illustration of the sheath system of FIG. 19 coupled to the cranial screw of FIG. 18, with a surgical system deployed through the sheath system.

FIG. 24 is an illustration of an example of the catheter of FIG. 19.

FIG. 25 is a schematic illustration of another non-limiting example of a guidance system, which is a specific implementation of the guidance system is as described in FIG. 2.

FIG. 26 is a schematic illustration of another example of a guidance system, which is a specific implementation of the guidance system as described in FIG. 2.

FIG. 27 is front view illustration of a specific implementation of the guidance system of FIG. 25.

FIG. 28 is a process schematic, which can be implemented using the previously described guidance systems.

FIG. 29 is an illustration of the implementation of a step within the process of FIG. 28 to generate a 3D surface model of the head of the subject.

FIG. 30 is an illustration of an example of the implementation of another two steps within the process of FIG. 28, where an optical marker has been placed on the subject, and the guidance system tracks the subject using the optical marker.

FIG. 31 is an illustration of an example of the implementation of another step within the process of FIG. 28, where the subject has been draped, an optical marker has been placed on the subject, and the light pattern has been illuminated.

FIG. 32 is an illustration of an example of an alternative implementation of the step of FIG. 31, where the subject has been draped, an optical marker has been placed on the subject, and the image scene has been projected to the surgeon.

FIG. 33 is a side view of a pivotable cranial screw.

FIG. 34 is a cross-sectional view of the pivotable cranial screw of FIG. 33, illustrated with locking members.

FIG. 35 is a perspective view of the pivotable cranial screw of FIG. 35, components within the cranial screw, and a cap configured to be coupled to the pivotable cranial screw.

FIG. 36 is an illustration of the guidance system of FIG. 27 and the pivotable cranial screw of FIG. 35.

FIG. 37 is a schematic illustration of the sheath system of FIG. 19 interfacing with the pivotable cranial screw of FIG. 35.

FIG. 38 is one schematic illustration of a surgical scaffold in a compressed state and another illustration of the surgical scaffold in an expanded state.

FIG. 39 is one schematic illustration of another surgical scaffold in a compressed state and another illustration of the surgical scaffold in an expanded state.

FIG. 40A is a schematic illustration of an expandable catheter in a compressed state.

FIG. 40B is a schematic illustration of an expandable catheter in an expanded state.

FIG. 40C is a schematic illustration of a top view of the expandable catheter of FIG. 40B in the expanded state.

FIG. 41 is an isometric view of an example of a robotic system for deployment of a surgical scaffold for ventricle procedures.

FIG. 42 is another isometric view of the robotic system of FIG. 41 with a portion of the housing of the robotic assembly removed for visual clarity.

FIG. 43 is a schematic illustration of a simplified cross-sectional view of the robotic system of FIG. 41 taken along the axial axis of FIG. 42.

FIG. 44 is a schematic illustration of a top view of another advancer that can be replaced with the advancer of the robotic system of FIG. 41 to advance the expandable catheter.

FIG. 45 is a schematic illustration of a robot arm.

FIG. 46 is an example of a flowchart of a process for implementing a neurosurgical procedure.

FIG. 47 are illustrations of an anterior view and a lateral view of a ventricle of a brain of a subject prior to the deployment of any surgical system.

FIG. 48 are illustrations of an anterior view and a lateral view of the ventricle of the brain of the subject of FIG. 47 with an expandable catheter deployed in a compressed configuration.

FIG. 49 are illustrations of an anterior view and a lateral view of the ventricle of the brain of the subject of FIG. 48 with the expandable catheter just prior to expansion.

FIG. 50 are illustrations of an anterior view and a lateral view of the ventricle of the brain of the subject of FIG. 48 with the expandable catheter in an expanded configuration.

FIG. 51 are illustrations of an anterior view and a lateral view of the ventricle of the brain of the subject of FIG. 48 with a surgical scaffold that is in the compressed configuration and that is positioned within the expandable catheter that is in the expanded configuration.

FIG. 52 are illustrations of an anterior view and a lateral view of the ventricle of the brain of the subject of FIG. 48 with a surgical scaffold that is in the expanded configuration and that is positioned within the expandable catheter that is in the expanded configuration.

FIG. 53 are illustrations of an anterior view and a lateral view of the ventricle of the brain of the subject of FIG. 48 with a surgical scaffold that is in the expanded configuration and that is positioned within the expandable catheter that is in the expanded configuration. An ultrasonic aspirator has been advanced through and past the surgical scaffold that is in the expanded configuration.

FIG. 54 are illustrations of an anterior view and a lateral view of a ventricle of the brain of a subject with another expandable catheter that is a ventricular catheter that is in an expanded configuration.

FIG. 55 are illustrations of an anterior view and a lateral view of the ventricle of the brain of the subject of FIG. 54 with a second instrument that is deployed through the expandable catheter that is in the expanded configuration.

FIG. 56 is an image of a display of a digitally rendered 3D mask and a 3D model of the subject's head.

FIG. 57 is an image of a display of a post-procedure CT image, verifying the placement of the catheter through the stylet and cranial screw of FIG. 59.

### DETAILED DESCRIPTION

As noted above, placement of an EVD to treat a CSF obstruction is typically completed manually, using a freehand technique. More specifically, the surgeon visually locates surface landmarks to determine an entry point to ultimately place the EVD device. Unfortunately, this placement can be relatively blind and unreliable, as the surgeon cannot definitively know the tissue underneath the entry point. Not surprisingly, EVD placement is typically accompanied by multiple complications, which can include catheter misplacement, injury to surrounding brain tissue, intracerebral hemorrhage, and sometimes even death. The worst complication, aside from death, is massive intracranial hemorrhage, typically from injury to an intracranial blood vessel (e.g., a vein), during placement. Additionally, in some cases, multiple failed attempts when placing the EVD during a blind procedure can result in injury to normal brain tissue. Unfortunately, these above-mentioned complications are widely recognized and even accepted.

Interestingly, even when considering the above complications, the placement of an EVD is presumed to be a simple and straightforward procedure. In fact, EVD placement is completed not just in a neurosurgical setting, but also in various other hospital settings including the emergency room, the critical care setting, in the operating room, and most commonly in the Intensive Care Unit (“ICU”) setting. Thus, it is not surprising that the number of EVD placement complications is relatively high. A recent study investigated the hemorrhagic risk by evaluating possible repercussions from implementing traditionally accepted trajectories. The study evaluated the traditional trajectory against imaging of cortical vascular structures (e.g., by using computed tomographic angiograms with venogram (“CTA/CTV”) data) to identify whether, or to what extent, the current trajectory could intersect a cortical vascular structure. Ultimately, the study demonstrated that all vessel injuries may have been avoided by moving the trajectory less than 1.0 cm laterally and less than 1.0 cm along the anterior/posterior axis. This study, and others, suggest that empirical measures are suboptimal when placing an EVD.

As detailed above, typical placement of an EVD is likely to be sub-optimal, if not misplaced. Thus, the previously used trajectory (and entry points) to place an EVD is likely not utilized for subsequent surgeries through them. In other words, if the previously used trajectory was utilized for subsequent surgeries, further damage to the patient's adjacent brain tissue, or cortical vascular structures could be contacted and damaged. As such, a new trajectory (with a new entry point) is utilized for subsequent surgeries. This failure to utilize the previous trajectory introduces compounding problems. First, hydrocephalus is typically caused by other rapidly progressing disease states (e.g., a tumor, swelling, a brain hemorrhage, etc.). Thus, a failure to utilize the previous trajectory forces the surgeon to waste precious time to introduce a second trajectory. This heavy increase in operating time is especially detrimental for these rapidly progressing disease states. Thus, the faster a surgical intervention is implemented, the less likely corresponding complications arise. Second, the introduction of a second trajectory inherently introduces other problems. For example, introducing a second trajectory can damage cortical vascular structures and adjacent brain tissue. Thus, sometimes, both trajectories can independently damage cortical vascular structures and adjacent brain tissue, or in some cases, together can damage cortical vascular structures and adjacent brain tissue (e.g., where the second trajectory exacerbates problems created by the first trajectory). Furthermore, the number of placement attempts has been correlated with ventriculostomy-related infection, such as ventriculitis and meningitis. Overall, the neurosurgical field requires improved mechanisms for optimal catheter placement that avoids both tissue and vascular injury with a single pass.

Some previous approaches have attempted to improve placement of an EVD by using highly sophisticated optical image guidance. Although these systems have been available and may improve current traditional methods, they are rarely used. In fact, these sophisticated guidance systems are extremely complex, and thus cannot be realistically utilized in the emergency setting. For example, these systems require both excessive time, and resources (e.g., equipment, assistance, etc.), which is unavailable for the emergency scenario. As another example, these navigation systems are extremely expensive, often times costing well over one million dollars. Thus, it is not surprising that these systems have not been widely implemented. As a result, these guidance systems have not had any significant influence on improving EVD placement.

Considering the above problems and lack of available solutions, there is a real need for a novel system for placing EVD catheters. More specifically, there is a need for a cost-effective approach that can be implemented in the emergent setting.

Some non-limiting examples of the disclosure can provide improvements over previous approaches, while addressing problems not considered, or ultimately introduced by the previous approaches. For example, some systems and methods according to some non-limiting examples, can determine patient-specific trajectories based on patient-specific structures. More specifically, CTA/CVA (or other imaging modalities) imaging data (or images) can be utilized and inputted into a trajectory determination system that determines a custom, patient-specific trajectory to accurately place the catheter. The custom, patient-specific trajectory avoids intersecting cortical vessels, which lessens the risk of intracranial hemorrhage.

To realize the custom, patient-specific trajectory, some non-limiting examples of the disclosure provide systems and methods that translate the custom, patient-specific trajectory into hardware, allowing for the improved placement of the catheter. For example, in some non-limiting examples, a 3D model of a mask is generated, which includes a bore aligning with the custom, patient-specific trajectory. In this case, when the 3D model of the mask is 3D printed, and the subject is wearing the 3D printed mask, a cranial screw can be placed into the bore, such that the cranial screw aligns with the custom, patient-specific trajectory. Other non-limiting examples of the disclosure can realize the custom, patient-specific trajectory in other ways. For example, rather than a 3D model of a mask, the custom trajectory can be transmitted to a guidance system that at least includes a projector and a camera. The camera can image the subject and generate a 3D surface model of the subject's head, which can be used and co-registered with a previously generated 3D model of the subject's head. The projector can then project an illumination pattern, which aligns with the custom trajectory. A pivotal cranial screw can be inserted at the custom entry point, and pivoted until a portion of the screw aligns with the custom trajectory.

Regardless of the methodology used to realize the custom, patient-specific trajectory, once the EVD (e.g., a catheter) is placed, the catheter can be subsequently used for other surgical interventions. For example, due to the catheter aligning with the custom trajectory, the surgeon knows that that the custom trajectory is likely not prone to introduce other problems or complications. Thus, the EVD can be utilized for other surgical interventions. Considering that CSF obstruction is likely caused by disease states requiring surgical intervention, the dual-ability of the EVD is especially appealing.

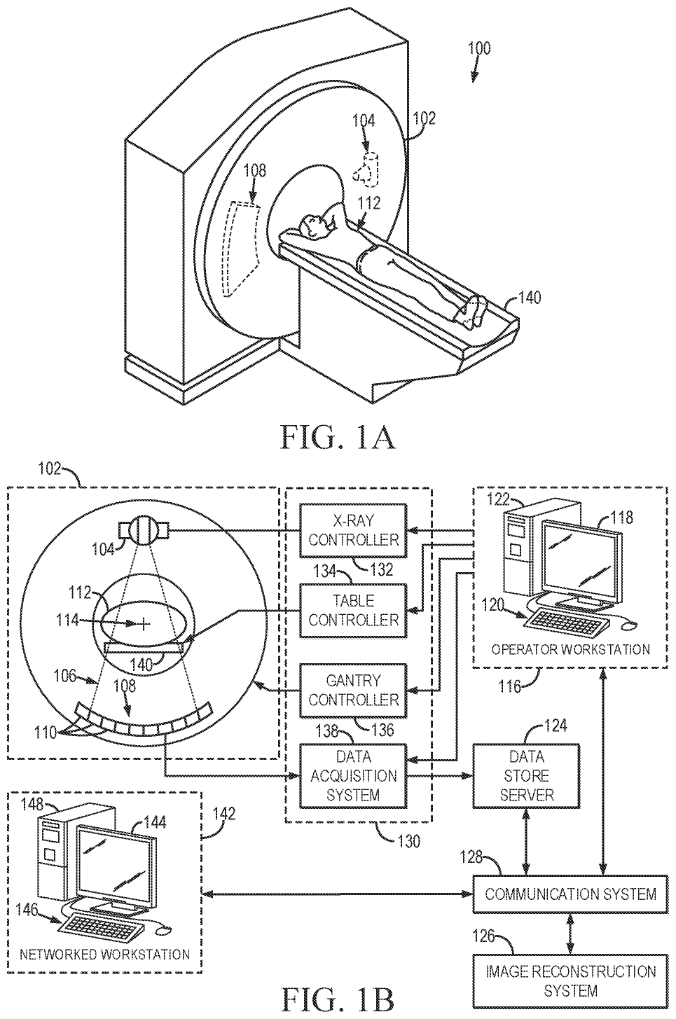

FIGS. 1A and 1B show an example of an x-ray computed tomography (“CT”) imaging system 100. The CT system includes a gantry 102, to which at least one x-ray source 104 is coupled. The x-ray source 104 projects an x-ray beam 106, which may be a fan-beam or cone-beam of x-rays, towards a detector array 108 on the opposite side of the gantry 102. The detector array 108 includes a number of x-ray detector elements 110. Together, the x-ray detector elements 110 sense the projected x-rays 106 that pass through a subject 112, such as a medical patient or an object undergoing examination, that is positioned in the CT system 100. Each x-ray detector element 110 produces an electrical signal that may represent the intensity of an impinging x-ray beam and, hence, the attenuation of the beam as it passes through the subject 112. In some configurations, each x-ray detector 110 is capable of counting the number of x-ray photons that impinge upon the detector 110. During a scan to acquire x-ray projection data, the gantry 102 and the components mounted thereon rotate about a center of rotation 114 located within the CT system 100.

The CT system 100 also includes an operator workstation 116, which typically includes a display 118; one or more input devices 120, such as a keyboard and mouse; and a computer processor 122. The computer processor 122 may include a commercially available programmable machine running a commercially available operating system. The operator workstation 116 provides the operator interface that enables scanning control parameters to be entered into the CT system 100. In general, the operator workstation 116 is in communication with a data store server 124 and an image reconstruction system 126. By way of example, the operator workstation 116, data store server 124, and image reconstruction system 126 may be connected via a communication system 128, which may include any suitable network connection, whether wired, wireless, or a combination of both. As an example, the communication system 128 may include both proprietary or dedicated networks, as well as open networks, such as the internet.

The operator workstation 116 is also in communication with a control system 130 that controls operation of the CT system 100. The control system 130 generally includes an x-ray controller 132, a table controller 134, a gantry controller 136, and a data acquisition system 138. The x-ray controller 132 provides power and timing signals to the x-ray source 104 and the gantry controller 136 controls the rotational speed and position of the gantry 102. The table controller 134 controls a table 140 to position the subject 112 in the gantry 102 of the CT system 100.

The DAS 138 samples data from the detector elements 110 and converts the data to digital signals for subsequent processing. For instance, digitized x-ray data is communicated from the DAS 138 to the data store server 124. The image reconstruction system 126 then retrieves the x-ray data from the data store server 124 and reconstructs an image therefrom. The image reconstruction system 126 may include a commercially available computer processor, or may be a highly parallel computer architecture, such as a system that includes multiple-core processors and massively parallel, high-density computing devices. Optionally, image reconstruction can also be performed on the processor 122 in the operator workstation 116. Reconstructed images can then be communicated back to the data store server 124 for storage or to the operator workstation 116 to be displayed to the operator or clinician.

The CT system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 116, or in a different facility, such as a different healthcare institution or clinic.

The networked workstation 142, whether within the same facility or in a different facility as the operator workstation 116, may gain remote access to the data store server 124 and/or the image reconstruction system 126 via the communication system 128. Accordingly, multiple networked workstations 142 may have access to the data store server 124 and/or image reconstruction system 126. In this manner, x-ray data, reconstructed images, or other data may be exchanged between the data store server 124, the image reconstruction system 126, and the networked workstations 142, such that the data or images may be remotely processed by a networked workstation 142. This data may be exchanged in any suitable format, such as in accordance with the transmission control protocol (“TCP”), the internet protocol (“IP”), or other known or suitable protocols.

The utilization of the CT imaging system 100 is only intended to be an example. The CT imaging system 100 shows, in one particular example, how imaging data may be obtained from a subject. However, in alternative non-limiting examples, other imaging modalities may be used. For example, imaging data used in the description below, may be derived from magnetic resonance imaging (“MRI”), x-ray imaging, ultrasound imaging, positron emission tomography (“PET”), or other imaging systems known in the art.

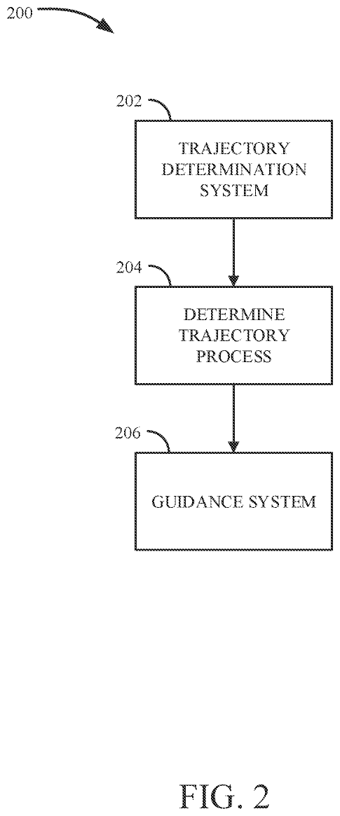

FIG. 2 shows a generalized system and process architecture 200 for improved EVD placement. The architecture 200 includes a trajectory determination system 202, a determine trajectory process 204, and a guidance system 206. The trajectory determination system 202 can embody many different forms. For example, the trajectory determination system 202 can be a computing device (e.g., the networked workstation 142), a server, or simply a processor. In some cases where the trajectory determination system 202 is implemented as a computing device, the computing device can include typical components used in the art to interact with the computing device, such as, for example, a display (e.g., the display 144), an input device (e.g., the input devices 146), a keyboard, a mouse, a graphical user interface, a touch-screen display, etc.

The determine trajectory process 204 is implemented on the trajectory determination system 202, which will be discussed in more detail below. Generally, the trajectory determination system 202 is used to implement the determine trajectory process 204, which when completed, determines the custom, patient-specific trajectory of the EVD. The guidance system 206 utilizes and realizes the determined patient-specific trajectory in various ways. For example, in some non-limiting examples, the guidance system 206 can include a three-dimensional (“3D”) printer that receives from the trajectory determination system 202 a patient-specific 3D model of the mask that includes the custom, patient-specific trajectory (e.g., in the form of a bore through the mask). In this implementation, the guidance system 206 can include a straight cranial screw that when interfacing with the mask, aligns with the custom trajectory on the patient.

As another example, the guidance system 206 can include a camera and a projector, among other components. The camera can image the subject to generate a 3D surface model of the subject. The 3D surface model of the subject can be co-registered with a previously generated 3D model of the subject (e.g., from an imaging modality). In some cases, the guidance system 206 can be tracked relative to the subject from the co-registration of anatomical features between the 3D models. The guidance system 206 can receive the custom trajectory coordinates and move the guidance system 206 to align the projector with the custom trajectory. Once sufficiently aligned, the projector can generate an illumination pattern onto the subject that aligns with the custom trajectory. In this implementation, the guidance system 206 can include an optical marker that can be placed on the subject. The optical marker allows for effective tracking by the camera, even when anatomical features used in registration are hidden from view (e.g., during surgical draping). In this case, the guidance system 206 can include a pivotal screw that the surgeon can implant at the illuminated entry point on the subject. Once implanted, a pivotal portion of the screw can be aligned with the illuminated trajectory, and can thus be locked in place.

As a further example, the guidance system 206 can include a camera and a projector. As discussed above, the camera can generate a 3D surface model, can co-register the 3D models, and can track the subject with the optical marker. In this implementation, the guidance system 206 can also include a augmented reality headset, which the surgeon can wear. The augmented reality headset can project the co-registered 3D model along with the custom trajectory. The augmented reality headset can be tracked relative to the subject using methods typically known in the art. This implementation can also include the previously discussed pivotal cranial screw, and a augmented reality hand interface component. The custom trajectory is projected to the surgeon and the hand interface component is tracked relative to the augmented reality headset. Thus, using the hand interface component in the same hand as the pivotal screw, the surgeon can implant the screw at the entry point, and can align and lock the pivotal portion of the screw to align with the custom trajectory.

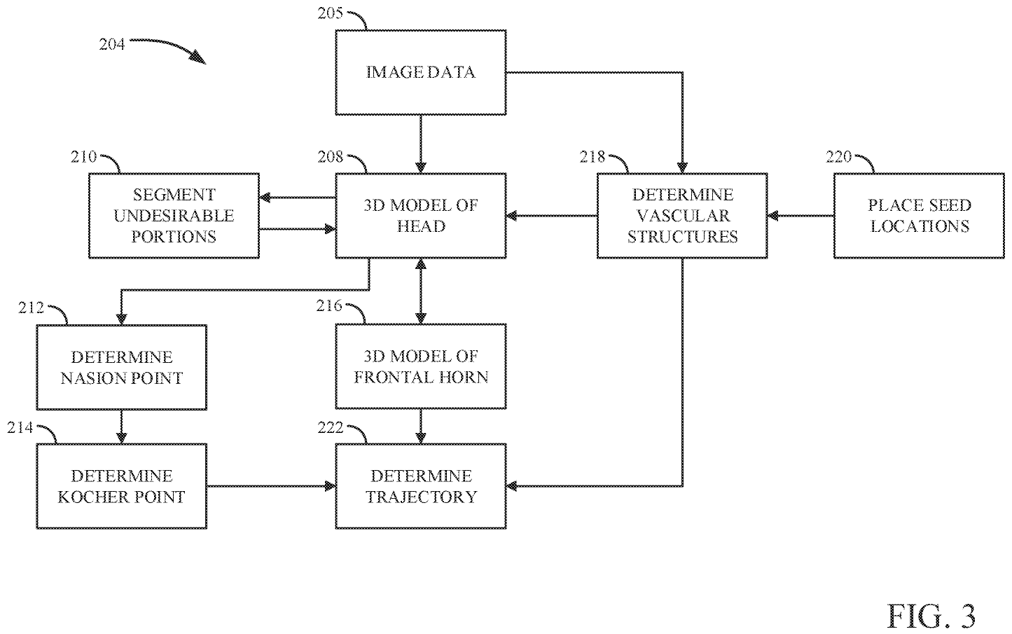

FIG. 3 is a schematic illustration of the determine trajectory process 204, which as discussed above, can be implemented on the trajectory determination system 202 (or one or more computing devices). The process 204 includes first acquiring imaging data 205. The imaging data 205 can be transmitted to the system 202 as pre-processed or post-processed (e.g., filtered, segmented, etc.), and in some cases, can be transmitted as pre-formed images in specific formats (e.g., Digital Imaging and Communications in Medicine (“DICOM”), etc.). The imaging data 205 is derived from the subject's head, and includes sufficient data (e.g., taken at multiple planes), such that 3D models of the subject's head can be generated. As indicated above, the imaging data 205 can be derived from various imaging modalities (e.g., CT, MRI, ultrasound, etc.), however, the remaining disclosure will describe the specific usage of CT imaging data as the image data 205.

Once the imaging data 205 is transmitted, a 3D model of the subject's head 208 is generated on the trajectory determination system 202. The 3D model of the subject's head 208 can be formed from typical processes known in the art (e.g., the marching cube algorithm). Similar methods can be used to model the nasion, frontal horn of the lateral ventricles, and intracranial vasculature. For example, a 3D model can be generated from at least two medical images taken at different planes, while in other cases the 3D model can be generated by adjoining together many slices of images taken along the same plane. The head surface can be segmented by applying a threshold filter followed by a fill-hole filter. The segmented head can then be converted to a 3D surface model using a marching cube algorithm. In some cases, once the 3D model 208 is generated, undesirable potions of the model can be segmented or removed at step 210. As a specific example, the trajectory determination system 202 can automatically remove at least a portion of the skull surface, while in other cases, a user can manually segment undesirable portions by interacting with a display (e.g., the display 144) and an input (e.g., the input devices 146). In some non-limiting examples, the user (or system 202) can ensure accurate generation of the model by placing original images (from the image data 205) over the model 208. The model 208 is typically visualized on the display as a surface model, where the 3D surface topology of the subject's head can be seen, and manipulated on the display (e.g., rotated).

After generation and sufficient segmentation of the 3D model of the head (e.g., steps 208 and 210), the trajectory determination system 202 can determine the Nasion point 212 on the model 208. This can be accomplished, for example, by locating the spatial position of the subject's eyes and nose from various projections. Then, as an example, the system 202 can determine the Nasion point as being the spatial location on the model 208 equidistant from the subject's eyes and at the minima of the nasal bone (as viewed from the side). In other non-limiting examples, a user can, via a user selection, select the Nasion point on the model 208 by interacting with a display and an input device. Once the Nasion point has been selected at 212 (e.g., automatically, or by a user selection), the system 202 can determine Kocher's point 214. In some cases, Kocher's point 214 is determined (via the system 202) by first generating a median plane that intersects the determined Nasion point (at 212). Then, the system 202 beginning at the Nasion point and moving backwards towards the back of the head, traverses the outer surface of the model 208 until a path length reaches 10 centimeters (or a different value). Then, the system 202 traverses the surface of the model 208 in a direction perpendicularly to the median plane, and leftwardly relative to the front view of the subject (e.g., the subject's right side) for three centimeters (or a different value). The system 202 then saves this coordinate as the Kocher's point and indicates this point with a fiducial marker on the model 208. In some cases, a user may interact with the model 208 (with the display) to select (using an input) Kocher's point, or to adjust the paths chosen, or planes chosen, by the system 202.

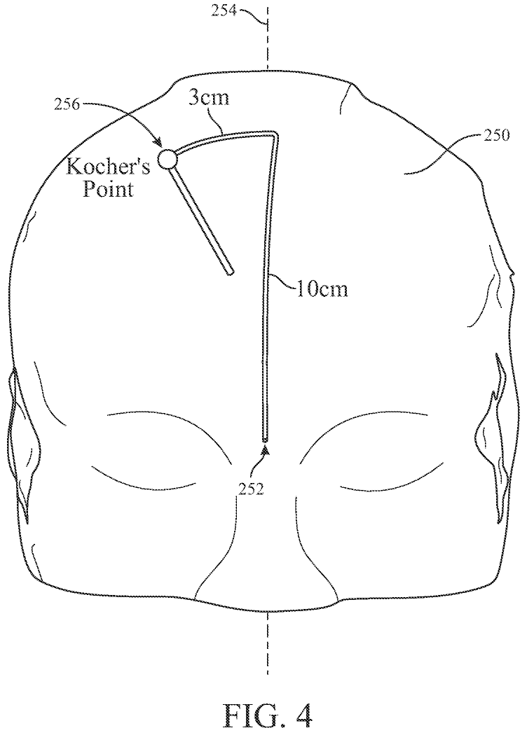

FIG. 4 shows an example of the determined Nasion and Kocher points on a 3D model of the head 250 (e.g., from step 208). As shown, after the Nasion point 252 has been determined and the coordinates saved, a median plane 254 is generated that intersects the Nasion point 252. Kocher's point 256 is determined relative to the median plane 254.

Referring back to FIG. 3, the 3D model of the frontal horn 216 can be determined by the system 202. In some cases, the system 202 can locate where the frontal horn resides within the model 208 by inputting slices (e.g., axial slices) into a machine learning model to segment the frontal horn out of the 3D model 208. A 3D model of the frontal horn can then be generated by compiling the slices of the frontal horn together. In other cases, the system 202 can generate the 3D model of the frontal horn 216 to be a generalized 3D shape aligning with a portion of the frontal horn (e.g., the general orientation of the frontal horn closest to the entry point). For example, a user can interact with the model 208 to select a transverse (axial) slice, which contains the frontal horn. Then, the user can select a dorsal point and a ventral point to define the bounds of the generalized 3D shape. The user can then adjust the thickness (e.g., perpendicular relative to the direction defined between the dorsal and ventral points). Once the points have been selected, the user can generate the generalized 3D shape. In some cases, the generalized 3D shape is a cylinder with a radius of 20 millimeters. In other cases, the generalized 3D shape can be a cone, with a decreasing cross-section as a reference point moves away from Kocher's point (e.g., a larger cross-section closer to Kocher's point). In other non-limiting examples, other generalized 3D shapes can be realized (e.g., a rectangular prism, a cube, a polyhedron, other 3D polygons, etc.). Once the 3D model of the frontal horn 216 is determined, the spatial position and size can be highlighted, and can be saved within the model 208.

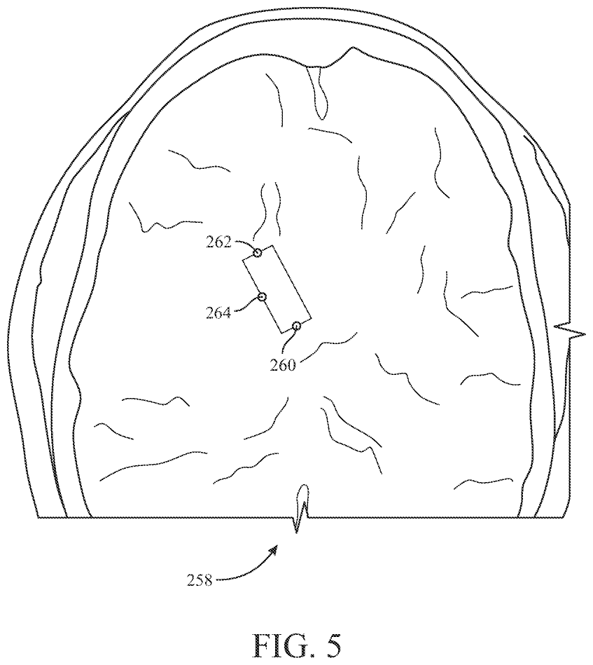

FIG. 5 shows an example of a user interacting with an axial slice 258 of an image of the subject, which in some cases, can be an axial slice of the model 208. As detailed above, a user can select a dorsal point 260 and a ventral point 262 within the axial slice 258, which defines the length of the 3D cylindrical model of the frontal horn (e.g., the 3D model 216). Then, the user can select a previously generated line spanning between the dorsal point 260 and the ventral point 262, indicated on FIG. 5 as reference point 264, to expand the width of the 3D cylindrical model of the frontal horn. In some cases, the system 202 requires that the 3D model 216 not exceed a particular volume. Thus, in this scenario, the system 202 may prevent further expansion of the width of the cylindrical model (e.g., when interacting with reference point 264). Once the desired dimensions of the 3D cylindrical model are selected, the system 202 can generate the 3D model 216 (e.g., by a user selection), and the dimensions and coordinates of the 3D model 216 (e.g., relative to the surface topology of the 3D model 208) can be saved within system 202.

Referring back to FIG. 3, the system 202 can determine cortical vascular structures at step 218. In some cases, a user can manually interact with imaging slices (e.g., from many imaging slices taken from the same plane throughout the entirety of the subject's head), or with slices of the model 208 (which can include images within its surface topology). The user can, via the system 202, highlight the boundaries of the vascular structures within each slice, and the system 202 can fill in the area for each slice, between the indicated boundaries. The system 202 can then generate a 3D model of the vascular structures by adjoining the areas (with a predefined imaging thicknesses) of adjacent slices. The 3D model of the vascular structures can be saved (or updated) within the 3D model 208.

In an alternative approach, a user can place one or more seed locations at step 220 within an imaging slice (or slice of the model 208) at portions of vascular structures within the imaging slice. Then, upon a user selection (or automatically), a machine learning model (stored on system 202, or in communication with system 202) can segment neighboring vascular structures (e.g., cortical veins) by utilizing a region growing process from the seed locations. Once the area within each image slice has been determined, and upon a user selection (or automatically), the system 202 can adjoin the adjacent areas (with a predefined imaging thickness) to generate a 3D model of the vascular structures. The dimensions and coordinates (e.g., relative to the surface topology of the 3D model 208) can be saved within (or updated within) the 3D model 208.

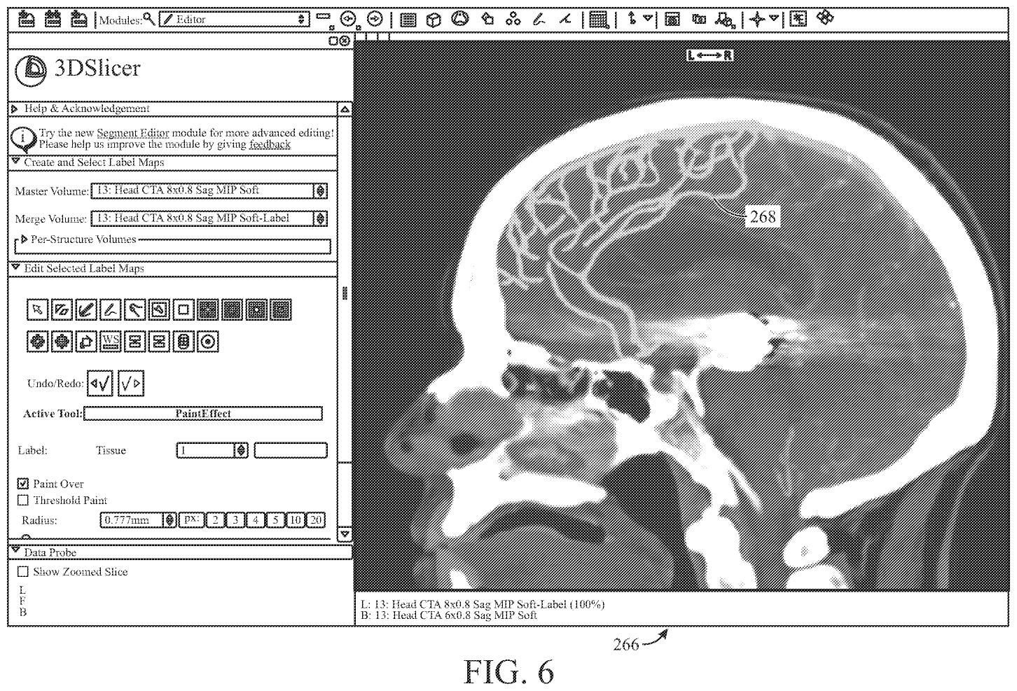

FIG. 6 shows an example of the machine learning model automatically identifying cortical veins. As shown, a sagittal plane CT slice 266 can be viewed on the display of the system 202. For this process, the vascular structures are defined using automatic vascular segmentation as a grant true in the convolutional neuronal networking (“CNN”). All images of the dataset are pre-processed by being skull-stripped and cropped to the frontal half of the head. The network is implemented using NiftyNet, a TensorFlow-based CNN platform. Training is performed on a graphics processing unit with an Adam optimizer with a learning rate of 1.0×10⁻³, batch size of 1, decay rate of 1.0×10⁻⁷, a maximal iteration of 10000, dice loss function, randomization of rotation in the range of −10° to 10° , and a window size of 512×192×96. Segmentations produced by the network are output as probabilistic label maps to allow for tuning of the result to be performed during later steps in the workflow by adjusting parameters for binning. Results are evaluated using Dice score, Hausdorff distance, specificity, and sensitivity, with preference given towards specificity over sensitivity owing to the nature of the use case. When completed, the machine learning model identifies the cortical veins pixels 268 for each slice, which can be used to generate the 3D model of the vascular structures (e.g., at step 218).

In some non-limiting examples, the system 202 can determine the trajectory at step 222 from various parameters. In some cases, the previously defined Kocher's point (e.g., at 214) is used by the system 202 as a starting entry point. Then, the system 202 determines a plurality of possible trajectories for the medical device. Each trajectory is a three-dimensional line, which intersects the 3D model of the frontal horn and which avoids intersecting with the 3D model of the vascular structures.

In some non-limiting examples, the system 202 can present to a user the trajectories, which avoid intersecting with the 3D model of the vascular structures by a threshold distance (e.g., 2 centimeters, 1 centimeter, etc.). For example, suppose a point on a given trajectory was less than the threshold distance. Then, this particular trajectory would be hidden (or removed) from view on the display. In some non-limiting examples, the trajectory is assumed as having a specific three-dimensional thickness (e.g., each trajectory is an idealized cylinder), which can be used to determine the plurality of trajectories. Generally, the trajectories can be determined due to the known spatial relationship between structures within the 3D model 208 (e.g., the 3D model of the vascular structures, the 3D model of the frontal horn, the surface topology of the 3D model 208).

After the plurality of possible trajectories are determined, the trajectories can be refined until a single, final trajectory remains. In some cases, a user can select the final trajectory from the plurality of trajectories by interacting with the display in communication with the system 202 (e.g., selecting the specific trajectory via a user input). In other cases, the final trajectory can be determined by optimizing specific parameters for the trajectories. For example, the system 202 can determine which trajectory, within the plurality of trajectories, has the largest distance between a reference point on the given trajectory and the 3D model of the vascular structures (where the reference point is the point on the given trajectory closest to the 3D model of the vasculature). In other words, the system 202 can determine a reference point on each trajectory that is closest to the 3D model of the vasculature. Then, the system 202 can select the trajectory with the greatest distance between the given reference point and the 3D model of the vasculature. As another example, the system 202 can select the final trajectory based on the closest distance between a reference point on a given trajectory and the Kocher's point. In some non-limiting examples, if desired, the user can compare the final trajectory with originally captured images from an imaging system (e.g., CT images) to validate the final trajectory. Once the user deems the final trajectory sufficient (or automatically in the case of the system 202's determination), the user can initiate a user selection that saves the coordinates, spatial position, etc., of the final trajectory relative to the model 208. Additionally, once saved, the trajectory can be displayed relative to the 3D model 208.

In some non-limiting examples, the system 202 can save the final trajectory and the corresponding information (e.g., the images, models, and patient sensitive information, etc.) related to the patient. In some cases, this information, such as information including the patient's name, sex, age, medical record number, and other protected health information (“PHI”) included in the image data will be removed from them before saving the data. The system 202, when saving the information, can apply a unique trajectory identifier for the specific case. In some scenarios, only the authorized personnel (e.g., the surgeon) can identify the patient by the trajectory identifier.

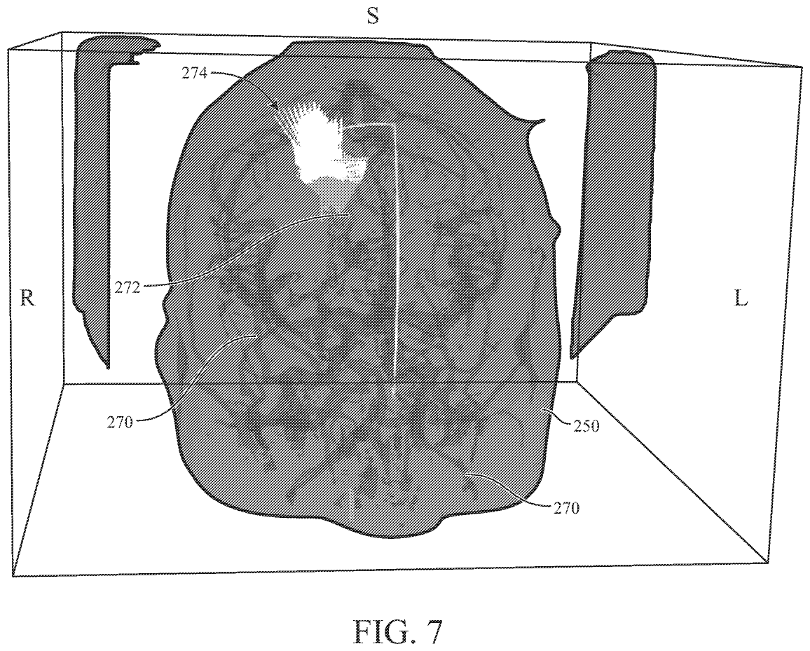

FIG. 7 shows a snapshot of a display of the 3D model 250 with a plurality of trajectories. As shown, the model 250 includes a 3D model of the vasculature 270 and a 3D model of the frontal horn 272, each disposed within the surface topology of the model 250. A plurality of trajectories 274 are displayed relative to the 3D model 250.

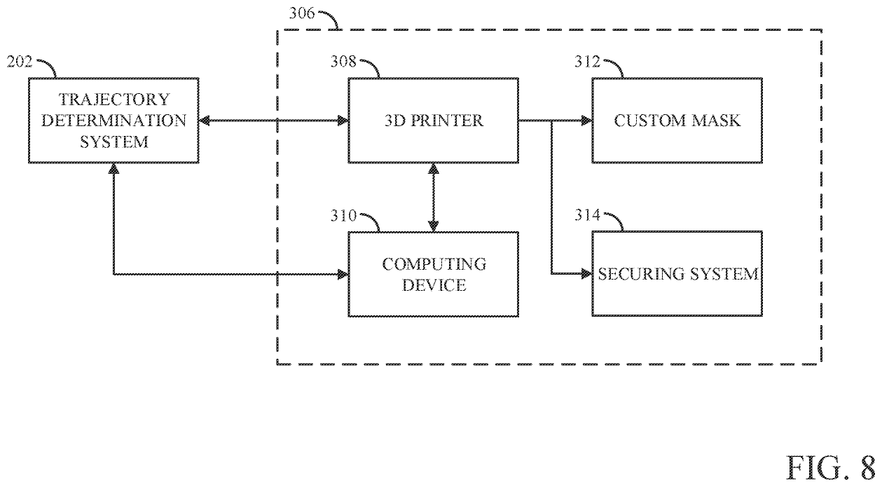

As discussed above, once the final trajectory is determined (e.g., using step 204), the dimensions, coordinates, spatial relationships, etc., between components and features can be transmitted and realized by the guidance system 206. In one non-limiting example, the guidance system 306 can be a specific implementation of the guidance system 206, as shown in FIG. 8. For example, the guidance system 306 can include a 3D printer 308, a computing device 310, a custom mask 312, and a securing system 314. The 3D printer 308 can include and can be typically structured as known in the art. For example, the 3D printer 308 can have a display, an extruder, a print plate, a processor (or controller), an extrudable material, etc. The 3D printer 308 is shown being in communication with the trajectory determination system 202 and the computing device 310. The computing device 310 can include components typically used in the art, such as, for example, a processor, memory, input(s), a display, etc. The computing device 310 is also shown being in communication (e.g., wired, wireless, serial, etc.) with the trajectory determination system 202 and the 3D printer 308. Generally, the securing system 314 functions to realize the final trajectory, as formed in the custom mask 312. For example, as will be discussed in more detail below, the securing system 314 can include screws (cranial), adapters, extension members, etc., so as to allow a catheter to align with the final trajectory as formed in the custom mask 312.

Once the trajectory determination system 202 has determined the final trajectory, the trajectory determination system 202 (or the computing device 310) digitally generates a custom, 3D patient specific mask 312. The digital custom, patient specific mask 312 contours the surface topology of the model 208, on at least a surface of the mask (e.g., the surface intended to interface with the patient). The boundaries of the mask 312 can be predetermined (e.g., set by the determination of anatomical features, such as the bridge of the nose), or can be adjusted and set by the user interacting with the system 202 (or the computing device 310). For example, the user can selectively adjust the dimensions of a cube that circumscribes the boundaries of the mask 312. Importantly, the boundaries of the mask 312 should be set, such that the custom mask 312 includes the entry point on the model 208 determined by the final trajectory. Thus, the custom mask 312 includes a bore that aligns with the final trajectory. In other words, the final trajectory performs an extruded cut on the custom mask 312 to generate the bore.

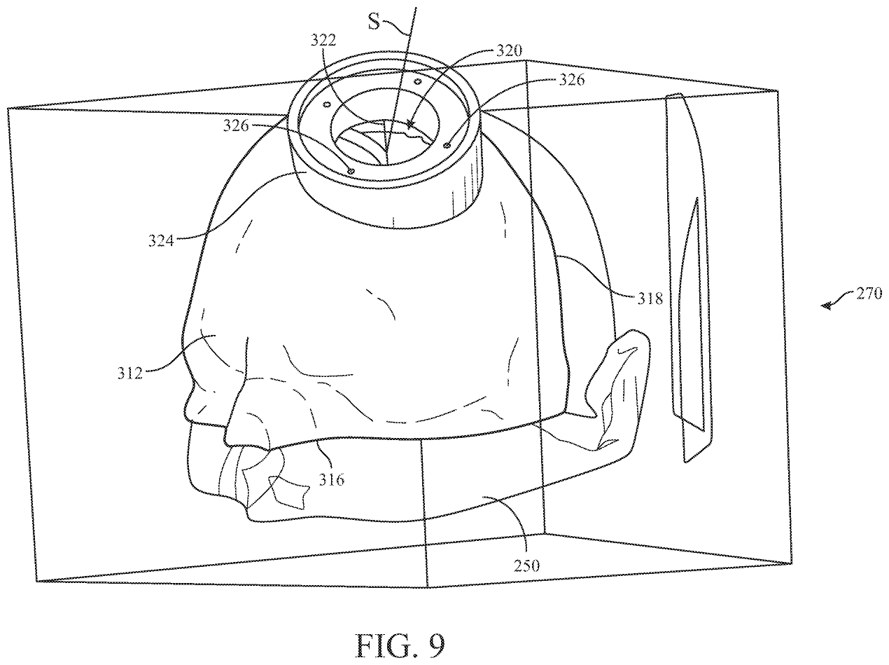

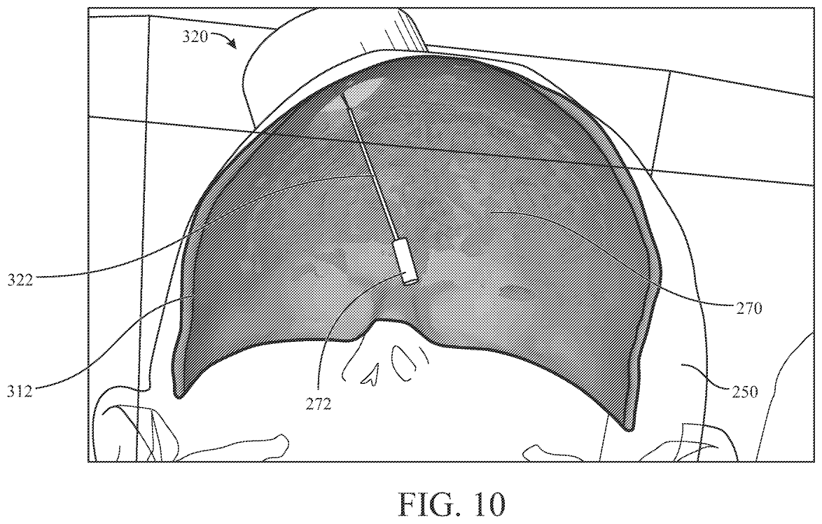

FIGS. 9 and 10 show a snapshot of a display of the 3D model 250 with the digitally rendered custom mask 312. As shown, the custom mask 312 has opposing surfaces that contour the surface topology of the 3D model 250. The custom mask 312 also includes edges 316, 318, which can be set, and restricted by the processes above. The custom mask 312 includes a bore 320 that aligns with the final trajectory 322. In the illustrated non-limiting example, the final trajectory 322 is idealized as a 3D cylinder having a specified radius, which can coincide with the radius of a typical EVD. The custom mask 312 can include an extruded cylinder 324 that surrounds the bore 320, and extends out of the custom mask 312 to align with the final trajectory 322. The extruded cylinder 324 can have holes 326 that are directed into a surface of the extruded cylinder 324, which can be dimensioned to receive fasteners (e.g., screws).

Once the system 202 has generated the digital, custom, patient specific mask, the system 202 (or the computing device 310) can transmit the dimensional, structural, data to the 3D printer 308 to create the custom mask 312. In some cases, the system 202 can communicate with the 3D printer 308 to determine the time required for printing the custom mask 312. For example, as detailed above, placing the EVD should be completed as soon as possible. Thus, the time required for printing the custom mask 312 should be completed under a threshold time (e.g., the time duration when the patient is being prepared for surgery, such as an hour). If the system 202 determines that the printing time is larger than the threshold time, the parameters of the mask 312, and the parameters of the 3D printer 308 can be adjusted to satisfy the printing time condition. For example, the dimensions of the mask 312 (e.g., the boundaries, thickness, etc.) can be adjusted, and the support material, extrusion movement, printing density of the 3D printer 308 can be adjusted.

In some non-limiting examples, for example when the practitioner has ample time, rather than 3D printing the custom mask 312, the custom mask 312 can be machined from a material (e.g., plastic, metal, etc.). For example, a computing device can transmit the 3D model of the digitally rendered mask 312 to a computer numeric control (“CNC”) machine for machining of the mask.

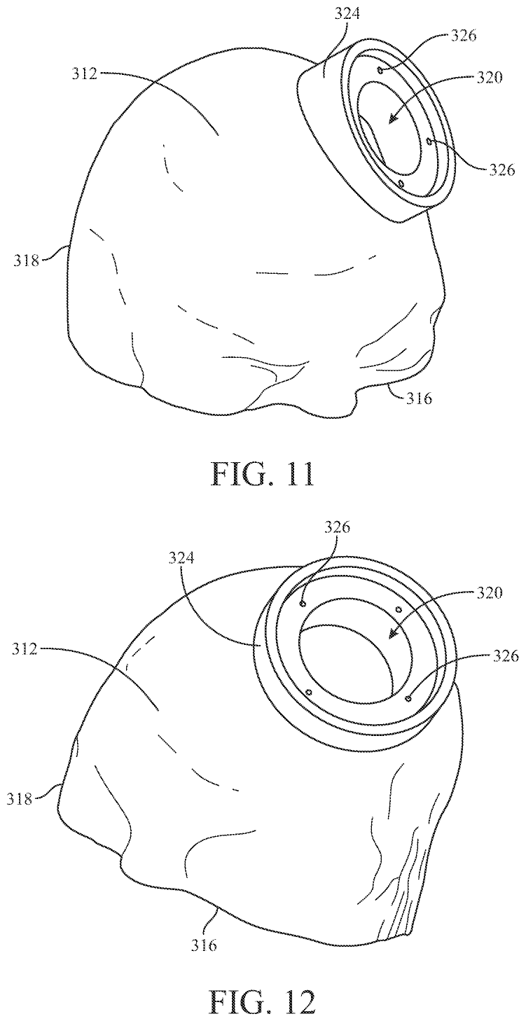

FIGS. 11 and 12 show the custom mask 312 after being 3D printed by the 3D printer 308. When the custom mask 312 is placed on the subject, the bore 320 will align with the previously determined final trajectory 322.

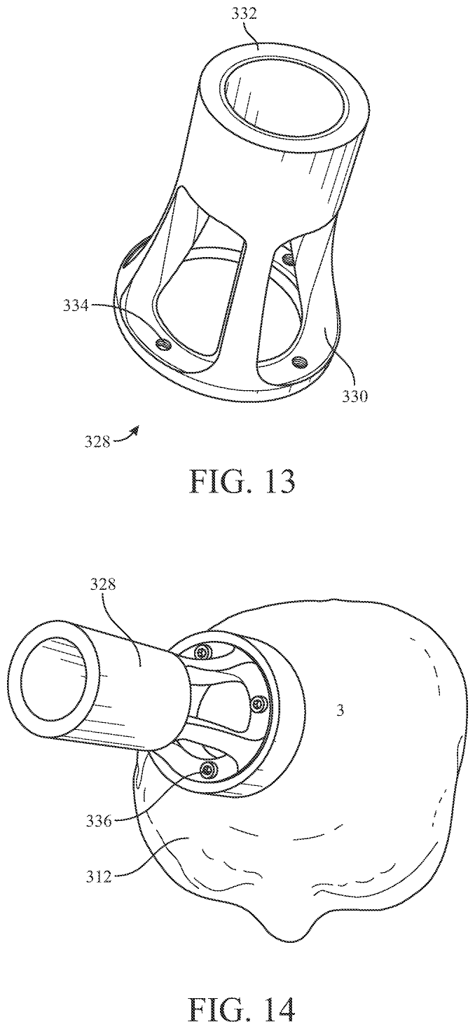

Referring back to FIG. 8, the custom mask 312 can include other interfacing components. For example, a mask attachment 328 shown in FIG. 13 can be coupled to the extruded cylinder 324 of the custom mask 312. More specifically, the mask attachment 328 includes two axially displaced circular members 330, 332. As illustrated, the lower circular member 330 and have apertures 334 directed through the lower circular member 330, which can correspond with the holes 326 of the mask 312. In some non-limiting examples, the dimensions of the circular member 330 (e.g., the radius) can correspond with the dimensions of the extruded cylinder 324. As shown, the mask attachment 328 includes an axial bore centrally directed into the mask attachment 328. Although the mask attachment 328 can be printed on the 3D printer 308, the dimensions of the mask attachment 328 are intended to be universal (e.g., the ability to be used with any custom mask).

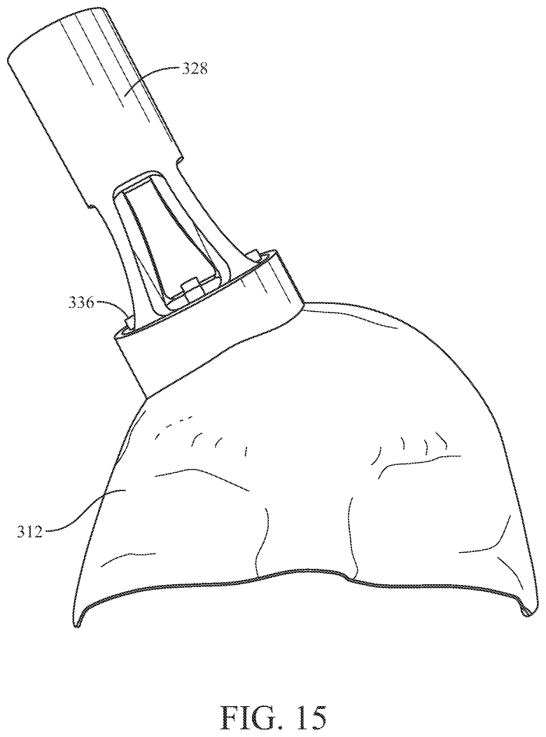

FIGS. 14 and 15 show the mask attachment 328 installed with the custom mask 312. As illustrated, the circular member 330 interfaces with the extruded cylinder 324 and the apertures 334 align with the holes 326. Then, each of the fasteners 336 (e.g., screws) can be received through an aperture 334 and a corresponding hole 326 to secure the mask attachment 328 to the custom mask 312. In some cases, the custom mask 312 can be installed with the mask attachment 328 prior to placement on the subject. In other cases, the mask attachment 328 is installed on the custom mask 312, after the custom mask 312 is placed on the subject.

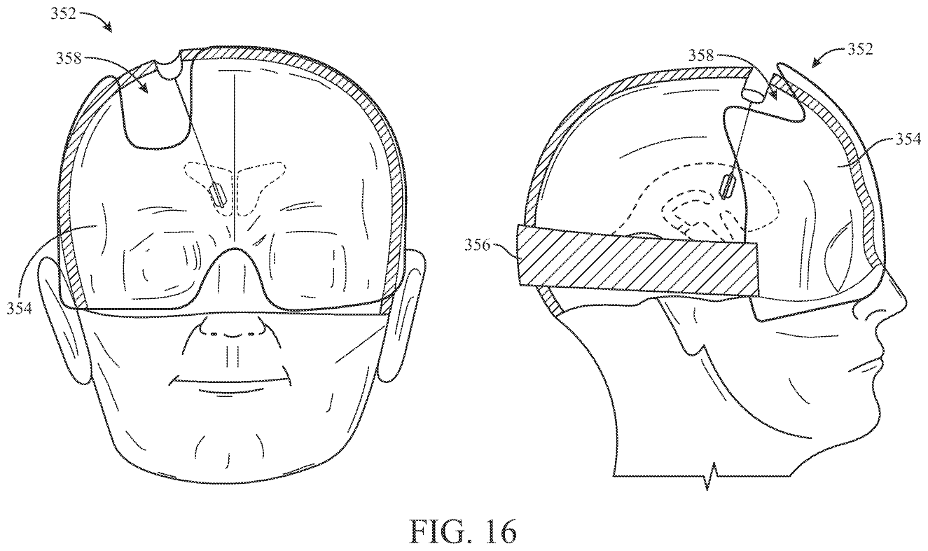

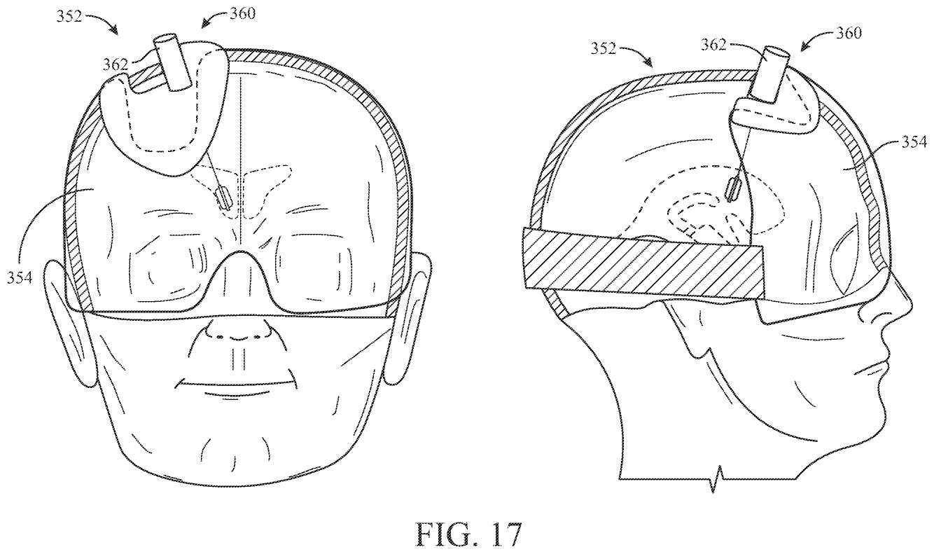

FIG. 16 shows a custom mask 352, which is an alternative non-limiting example of the custom mask 312. The custom mask 352 is formed in similar ways as the custom mask 312. Thus, the previous discussion of the custom mask 312 also pertains to the custom mask 352. Generally, the custom mask 352 has a main body 354 having at least one surface that conforms to the surface topology of the 3D model of the head. The mask 352 also includes a retaining strap 356, which can be coupled to the main body 354 following formation of the main body 354 (e.g., after 3D printing). In some cases, the retaining strap 356 can be slotted through the main body 354 and secured with hook and loop fasteners (e.g., on the retaining strap 356). The main body 354 includes a trajectory slot 358, which is dimensioned according to the final trajectory (e.g., the final trajectory 322). For example, the entry point defined by the final trajectory can be received within the boundary of the trajectory slot 358. Importantly, the mask 352 also includes a mask platform 360. The mask platform 360 is also dimensioned according to the final trajectory, such that when the mask platform 360 interfaces with the trajectory slot 358, a port 362 of the mask platform 360 aligns with the final trajectory. Although the trajectory slot 358 and the mask platform 360 are shown as having specific shapes, it can be appreciated that this is only intended to be an example. For example, the trajectory slot 358 and the mask platform 360 interface may be shaped differently, while still allowing the port 362 to align with the final trajectory.

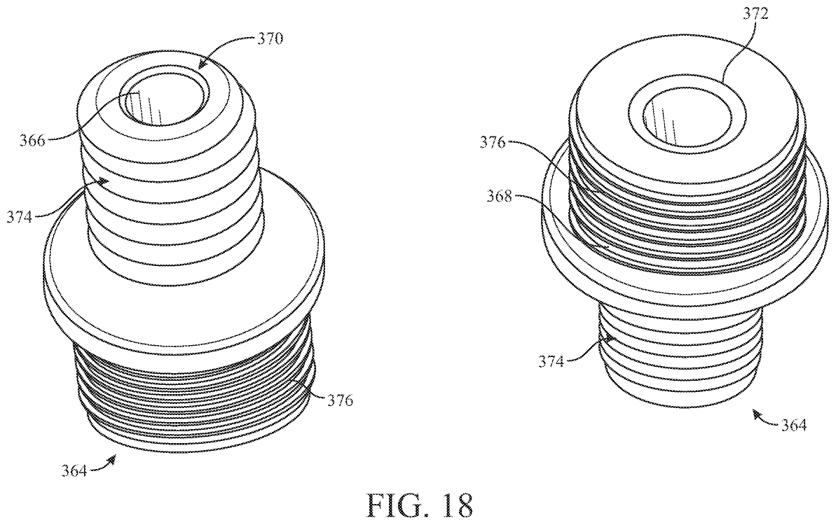

Once the custom mask has been created (e.g., the custom mask 312, 352), the custom mask is placed on the subject. Then, depending on the custom mask used, the bore 320 or the port 362 are used to guide the implantation of a cranial screw 364 (e.g., see FIG. 18), which can be a specific component of the securement system 314. More specifically, the bore 320 and the port 362 are dimensioned to receive the cranial screw 364. Thus, the skin on the head of the subject defined within the bore 320 (or the port 362) is excised away (e.g., with a scalpel). Then, the bore 320 (or the port 362) is used to guide a drill to create the cranial burr hole. Once sufficiently drilled, the cranial screw 364 is placed in the cranial burr hole.

As shown in FIG. 18, the cranial screw 364 includes an interior surface 366 and an opposing exterior surface 368. The internal surface 366, which includes a bore 370 that extends axially through the cranial screw 364 can include threads 372. The exterior surface 368 can have a first threaded region 374, which interfaces with the skull of the subject when implanted. Additionally or alternatively, in some non-limiting examples, the exterior surface 368 at the head 376 of the screw can include threads. Although, the head 376 of the cranial screw 364 is illustrated as being circular in shape, in other non-limiting examples the head 376 could be hexagonal (e.g., an a hexagonal prism) or other shapes to secure to other components of the securement system 314. Depending on the structure of a component of the securement system 314, the component can threadingly engage either or both of the threads (e.g., the threads 372) to secure to the cranial screw 364.

After the cranial screw 364 has been implanted, the custom mask can be removed and other components (e.g., included in the securement system 314), instruments, etc., can be installed. For example, to ensure that the catheter maintains the intended trajectory angle and depth, a guide wire can be housed within the catheter during placement to provide rigidity. Additionally, to ensure the catheter is inserted to the correct depth, there can be a stopper device that will prevent users from an insertion that is too shallow or too deep.

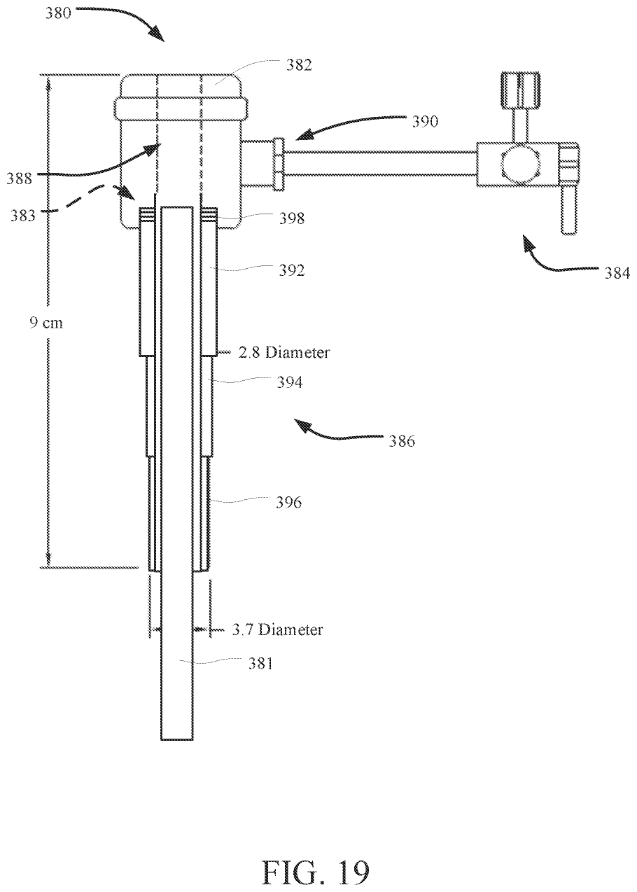

FIG. 19 shows sheath system 380, which is part of the securement system 314, and which is coupled to the cranial screw 364. The sheath system 380 includes a body 382, a side port 384, and a telescopic adapter 386 coupled to and extending from the body 382. The body 382 has two separate conduits 388, 390 although both conduits 388, 390 are in fluid communication with each other. Conduit 388 extends axially through the body 382 and allows for fluid communication through the telescopic adapter 386. This allows a catheter 381 to be inserted through the conduit 388, through the telescopic adapter 386, and through the axial bore 370 of the cranial screw 364. The conduit 390 extends generally perpendicularly relative to and interfaces with the conduit 388. Thus, the conduit 390 is also in fluid communication with the catheter 381. The conduit 390 is illustrated as interfacing with a side port 384, which can include components typically used in the art (e.g., valves, flow adjusters, etc.). Generally, a suction source (e.g., a pump) can be connected to the port 384 to draw CSF fluid out of the catheter 381, through the conduit 390 and out the port 384, which will be discussed in more detail below.

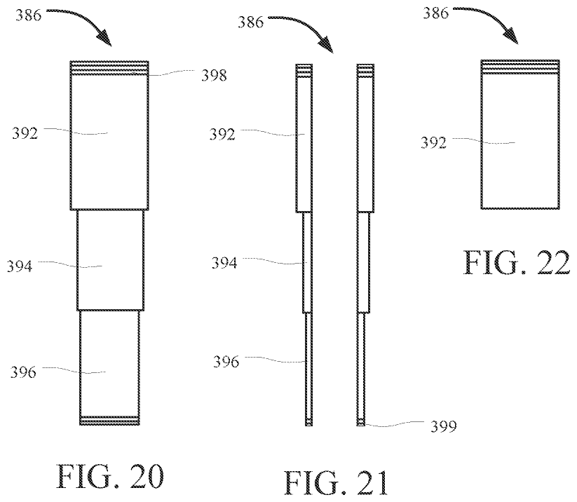

As shown in the illustrated non-limiting example of FIGS. 19-22, the telescopic adapter 386 includes telescoping members 392, 394, 396 that are cylindrical, which can be extended or retracted to adjust the total axial length of the telescopic adapter 386. The member 392 includes threads 398 that threadingly engage threads of a circular aperture 383 of the body 382. This can, for example, allow for the securement of the telescopic adapter 386 to the body 382, however, in alternative non-limiting examples, other components and methods for securement known in the art could be used. The member 394 is coaxially received within at least a portion of the member 392. Similarly, the member 396 is coaxially received within at least a portion of the member 394. Each of the members 392, 394, 396, may have circular flanges on respective ends, such as, to block further extension of a particular member.

As shown in FIG. 21, the member 396 has internal threads 399 that are configured to threadingly engage the threads 376 or 372 of the cranial screw 364, depending on the dimensions or structure of the member 396. This way, the telescopic adapter 386 can be secured to the cranial screw 364. Referring back to FIG. 19, the entire axial length of the telescopic adapter can be adjusted to accommodate for a particular length of the catheter 381 outside of the subject. For example, if the intracranial component of the EVD (e.g., catheter 381) is 6.5 centimeters (“cm”) from the skull surface, a fixed-length 15 cm EVD catheter will have 9.5 cm in extracranial length. Thus, the axial length of the telescopic adapter 386 can be adjusted to the remaining 7.5 cm. This can be accomplished in many different configurations. For example, in one configuration, the members 394 can be rotated to free an engagement between a protrusion (or axial slot) in the member 394 that locks with an axial slot (or protrusion) of the member 392. Then, the member 394 can be advanced or extended to the desired distance and rotated back to lock the member 394 relative to the member 392. Similarly, in this configuration, the member 396 can also be rotated to free an engagement between a protrusion (or axial slot) in the member 396 that locks with an axial slot (or protrusion) of the member 394. Then, the member 396 can be advanced or extended to the desired distance and rotated back to lock the member 396 relative to the member 394. In other non-limiting examples, other mechanisms are contemplated to lock the members together.

As another example, suppose the intracranial EVD depth was 5.5 cm. The axial length of telescopic adapter 386 could be adjusted to 8.5 cm. As a still further example, if the intracranial depth was 7.5 cm for the EVD, the axial length of the telescopic adapter 386 could be adjusted to 6.5 cm, etc. In some non-limiting examples, it is contemplated to have various dimensions of the members 392, 394, 396, and in some cases, additional members (e.g., 4, 5, 6, etc.) could be used. For example, the diameter of the bore for each of the members 392, 394, 396 can be at least the diameter of the catheter 381, such that the catheter 381 (and other surgical instruments) can be received through the telescoping adapter 386. As another example, the length of the member 392 can be shorter than the other members, so as to allow for increased adjustment for the axial length of the telescoping member 386. In some non-limiting examples, the axial adjustability of the telescoping member 386 prevents the need to cut the catheter 381. Typically, once a catheter has been placed, the portion exterior to the patient is cut. However, if cut too short, for example, the catheter can recede through the screw and must be retrieved. The adjustability of the telescoping member 386 prevents the need to cut the catheter, and thus mitigates some of the potential problems.

In some non-limiting examples, the telescoping member 386 is formed out of a rigid material (e.g., a metal, or hard plastic, etc.), to provide a rigid structure that aligns with the predetermined or final trajectory. This way, a guidewire (and the catheter 381) and other surgical instruments, are kept with alignment with the final trajectory.

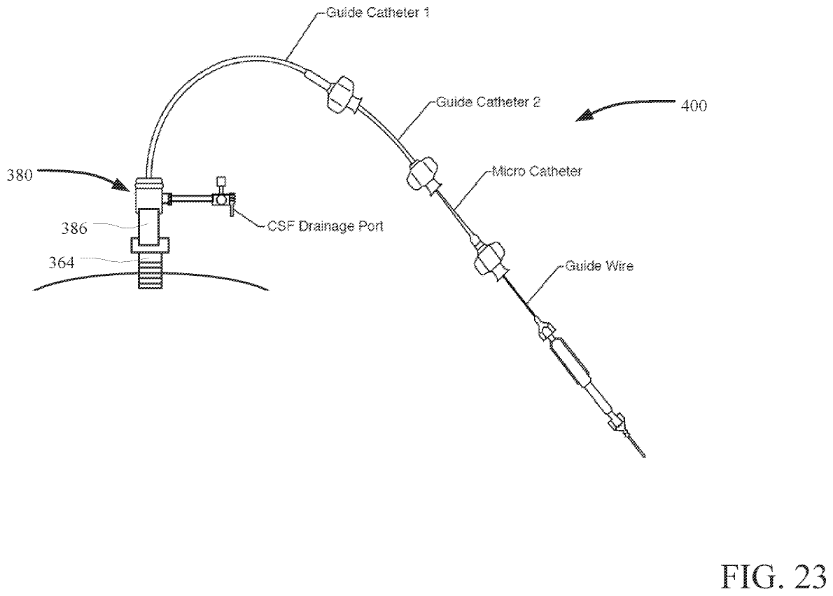

FIG. 23 shows the telescoping adapter 386 installed with the cranial screw 364 and the sheath system 380. In particular, one end of the telescopic adapter 386 is removably coupled to the cranial screw 364 and second opposite end of the telescopic adapter 386 is removably coupled to the sheath system 380 (e.g., the body 382 of the sheath system 380). After the cranial screw 364 has been implanted and secured, the axial length of the telescoping member 386 is adjusted to the particular length determined by the final trajectory. For example, in some non-limiting examples, a surgeon (or other practitioner) can determine (e.g., from the trajectory determination system) the intracranial depth of the catheter determined by the final trajectory. In some embodiments, the intracranial depth is defined by the magnitude of the 3D line that defines the final trajectory. Then, the surgeon (or other practitioner) can use the length of the catheter 381 (e.g., 15 cm), subtract from the intracranial depth, and adjust the axial length of the telescoping member 386 to the subtracted distance. Once the desired length has been achieved, the telescoping member 386 can be locked and secured to the cranial screw 364 (e.g., by threaded engagement between the threads 399 and the threads 376). Then, the body 382 can be secured to the telescoping member 386 (e.g., by threaded engagement between the threads 398 and the threads of the circular aperture 383). Subsequently, a surgical system 400 can be deployed through the conduit 388, into the telescoping member 386, through the bore 370 of the cranial screw 364, and into the subject.

In the illustrated non-limiting example, the surgical system 400 can include a first guide catheter, a second guide catheter coaxially received within the first guide catheter, a micro catheter coaxially received within the second guide catheter, and a guidewire coaxially received within the micro catheter. Generally, each component within the surgical system 400 is configured to be received through the conduit 388, through the telescoping adapter 386, and into the bore 370 of the cranial screw 364. In some non-limiting examples, once the catheter 381 is secured to the body 382, subsequent surgery can be completed through the catheter 381. In some non-limiting examples, the catheter 381 will come joined to the body 382 in what is effectively one piece. Thus, the sheath system 380 can be provided in an operative surgical equipment kit, such that the surgeon will not be required to do any assembly at the bedside (e.g., secure the catheter 381 to the body 382). For example, depending on the associated disease state, the surgical system 400 can deliver drugs (e.g., tumor suppressing drugs, cytotoxic agents, anticoagulants, etc.), and other instruments (e.g., excising instruments) to treat the underlying disease state.

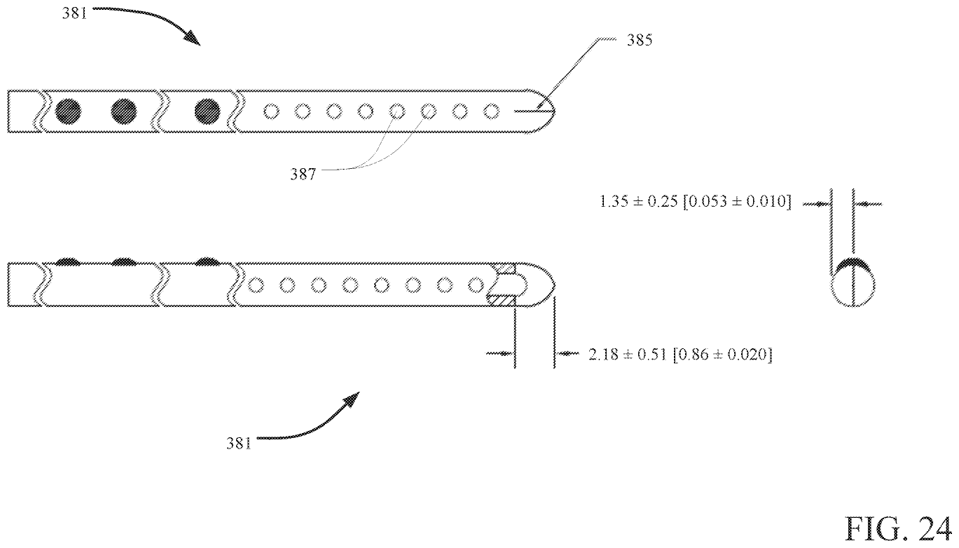

In some non-limiting examples, the catheter 381 must be sized appropriately to be able to receive the instruments within the surgical system 400. For example, the catheter must have a diameter wide enough to fit and advance the instruments. Additionally, as illustrated in FIG. 24, the catheter 381 can have a slit 385 on a distal end, such that an instrument within the surgical system 400 can advance through the distal end of the catheter 381. In alternative non-limiting examples, however, a distal end of the catheter 381 can have an open end, such that an instrument can advance through the distal end of the catheter 381. The catheter 381 can include a plurality of holes 387 directed through the catheter 381 and disposed on a distal end thereof. As shown, the plurality of holes 387 are aligned along an axial axis defined by an elongate direction of the catheter 381, and are aligned with the slit 385. Although only one lateral side of the catheter 381 has the plurality of holes 387, the opposing lateral side of the catheter 381 can include another plurality of holes. In some cases, each of these plurality of holes can be aligned with a corresponding hole of the plurality of holes 387. Although the holes 387 are illustrated as being circular in shape, in other configurations they can have different shapes (e.g., squares, rectangles, etc.). The holes 387 generally facilitate the drawing of CSF away from the ventricle and through the catheter 381.

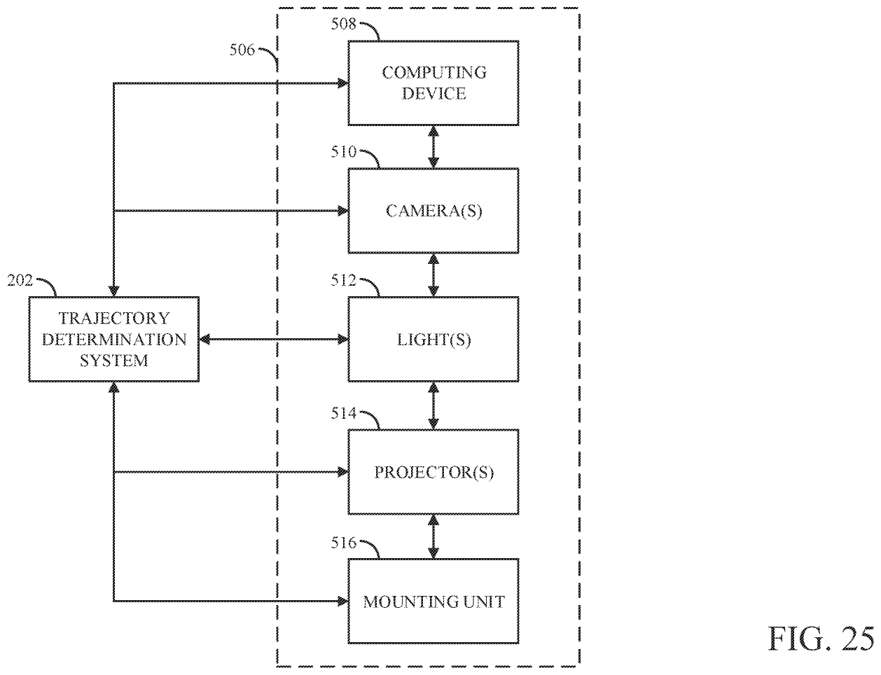

FIG. 25 shows in a second non-limiting example, the guidance system 506 being another implementation of the guidance system 206. For example, the guidance system 506 can include a computing device 508, cameras 510, lights 512, projectors 514, and a mounting unit 516. The computing device 508 can include components typically used in the art, such as, for example, a processor, memory, input(s), a display, etc. The computing device 508 is also shown being in communication (e.g., wired, wireless, serial, etc.) with the trajectory determination system 202, and all of the other components within the guidance system 506. The computing device 508 can embody the components previously discussed above with regard to the trajectory determination system 202 and the computing device 310. For example, the computing device 508 can be implemented simply as a processor. Generally, the computing device 508 can receive information from, and can instruct (or cause) components within the guidance system 506 to complete a particular task. As an example, the computing device 508 can instruct the cameras 510 to acquire images.

The cameras 510 can be typically structured as known in the art, including lenses, image sensors (e.g., a charge-coupled device, an active-pixel sensor, etc.), filters, etc. The following description of the cameras 510 will be described as being implemented as having two cameras, however in alternative non-limiting examples, a single, or different multiples of cameras can be used. The lights 512 can also be typically structured as known in the art. The lights 512 can be implemented as a light emitting diode (“LED”), an LED array, a bulb, etc. The lights 512 are configured to provide sufficient lighting, such that the cameras 510 can acquire images of sufficient clarity.

In some non-limiting examples, the projectors 514 can embody many different forms as typically used in the art. For example, the projector 514 can be implemented as an image projector, which can be configured to project an image onto an imaging plane. In other non-limiting examples, the projector 514 can be implemented as a typically structured laser (e.g., including a light (LED) and a gain medium, etc.). Additionally, in some cases, the projector 514 can include other optical components (e.g., lenses, mirrors, etc.), which can be electronically controlled (e.g., by the computing device 508). This way, the angle that the laser or image projection is emitted can be adjusted.

In some non-limiting examples, the mounting unit 516 mechanically couples all of the components within the guidance system 506 together. For example, the mounting unit can have a housing, which is structured to receive the computing device 508, the cameras 510, the lights 512, and the projectors 514. The mounting unit 516 can also have other movable mechanisms typically used in the art. For example, the mounting unit 516 can have a single actuatable joint (e.g., a pivotable joint having three rotational degrees of freedom) to orient the housing of the mounting unit 516 at a particular orientation. Additionally, the mounting unit 516 can include other actuatable joints to move the housing of the mounting unit 516 along the remaining three degrees of freedom. More specifically, the mounting unit 516 can have a first actuatable arm coupled to a first actuator, a second actuatable arm coupled to a second actuator, and third actuatable arm coupled to a third actuator. In this scenario, movement of the first, second, and third actuators in a particular order can orient the housing of the mounting unit 516 at a particular angle, and distance away from a reference object. Although the mounting unit 516 has been referenced with specific examples, it can be appreciated that the mounting unit 516 can include other robotic components to orient the housing of the mounting unit 516 at a particular angle, and a distance away from a reference object.

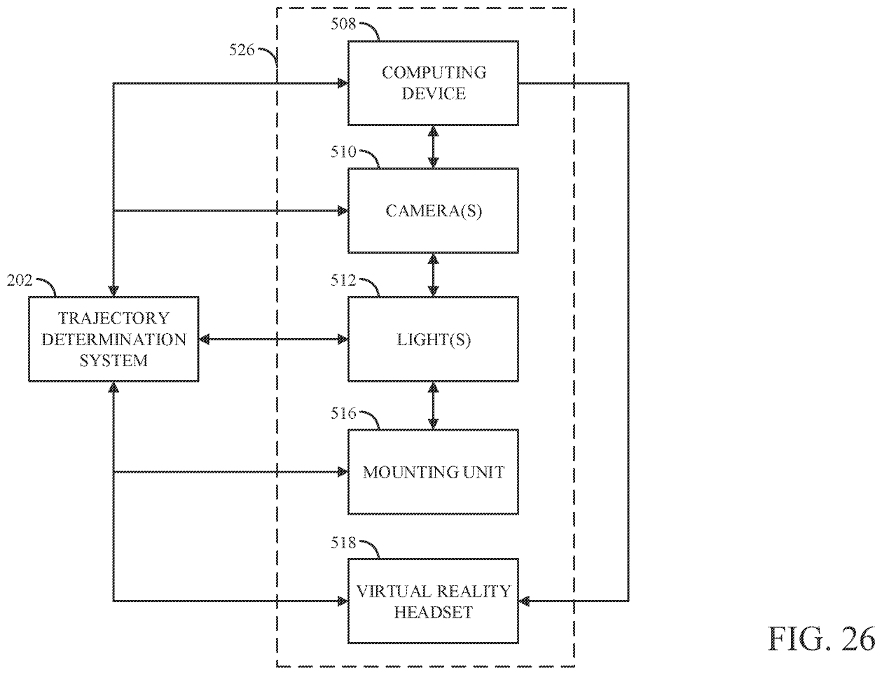

FIG. 26 shows a third shows in a third non-limiting example, the guidance system 526 being another implementation of the guidance system 206. The guidance system 526 includes many of components previously discussed with regard to the guidance system 506. Thus, what pertains to the guidance system 506 also pertains to the guidance system 526. One difference between the guidance system 506 and the guidance system 526 is the substitution of the augmented reality headset 518 for the projector 514. The augmented reality headset 518 can be include typical components, and can be typically structured as known in the art. For example, the augmented reality headset 518 can include a stereoscopic head-mounted display to project to a user a 3D image scene. Similarly, the augmented reality headset 518 can include motion tracking sensors (e.g., gyroscopes, accelerometers, magnetometers, etc.) to track the position and orientation of the augmented reality headset 518 relative to a reference object. Typically, the augmented reality headset 518 can include a processor (or other computational device) to implement the above functional characteristics (e.g., projecting images, tracking positions, etc.). As shown the augmented reality headset 518 is in communication with the computing device 508 and the trajectory determination system 202 (e.g., by wireless communication).

In some non-limiting examples, the augmented reality headset 518 can include wireless or tethered interfacing components. As an example, an interfacing component can be a glove that include sensors on the fingers (or fingertips), which can be tracked relative to the head mounting structure of the augmented reality headset 518. This way, ultimately the glove can be tracked relative to the reference object.

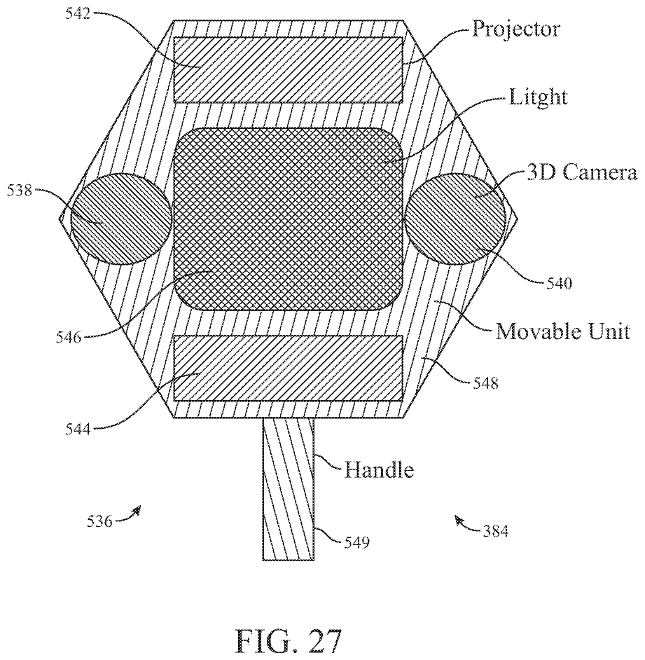

FIG. 27 shows an example of a guidance system 536, which is a specific implementation of the guidance system 526. The guidance system 536 includes cameras 538, 540, projectors 542, 544, a light 546, a housing 548, and a mounting unit 549. The cameras 538, 540, can be similar to the camera 510, the projectors 542, 544 can be similar to the projector 514, the light 546 can be similar to the light 512, and the mounting unit 549 can be similar to the mounting unit 516. As shown, the cameras 538, 540 are stereoscopically positioned on the housing 548, such that when images are acquired from the cameras 538, 540, a 3D image scene can be reconstructed, and the distances between objects (or the distance between the mounting unit 549 and an object) can be determined. Although in this non-limiting example, there are two projectors 542, 544, in alternative non-limiting examples there may be only a single projector. The following description will describe the two projectors 542, 544 being implemented as both an image projector, both lasers, or one being a laser and one being an image projector. In some non-limiting examples, although not illustrated in FIG. 27, the guidance system 536 can include a computing device (e.g., the computing device 508).

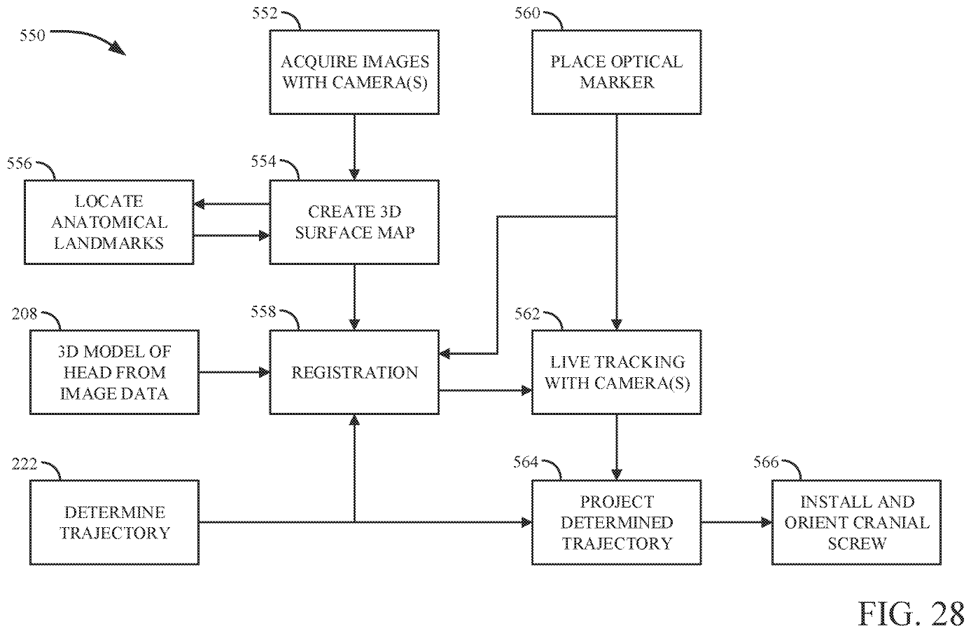

FIG. 28 shows a process 550 that can be implemented using any of the guidance systems 506, 526, and 536. Thus, the process 500 can be implemented using a computing device corresponding with the particular guidance system (or one or more computing devices). The process 550 can begin with acquiring images with cameras at step 552. In some cases where there is sufficient lighting, the cameras can acquire (e.g., via instruction by the computing device) images of the head of the subject necessary to generate a 3D model of the subject's head. For example, the mounting structure can be electrically actuated (e.g., via actuatable joints) to change the ordination and position of the mounting unit (or housing) relative to the subject's head, such that the cameras can capture images at different positions and orientations. This way, the cameras can generate a more accurate 3D representation of the subject's head. In other cases, rather than the mounting structure being electrically actuated, in some non-limiting examples, the user can change the orientation and the position of the mounting unit (or housing) relative to the subject's head to acquire images from different perspectives. In the cases where the lighting is insufficient, the light can be activated to illuminate the image scene.

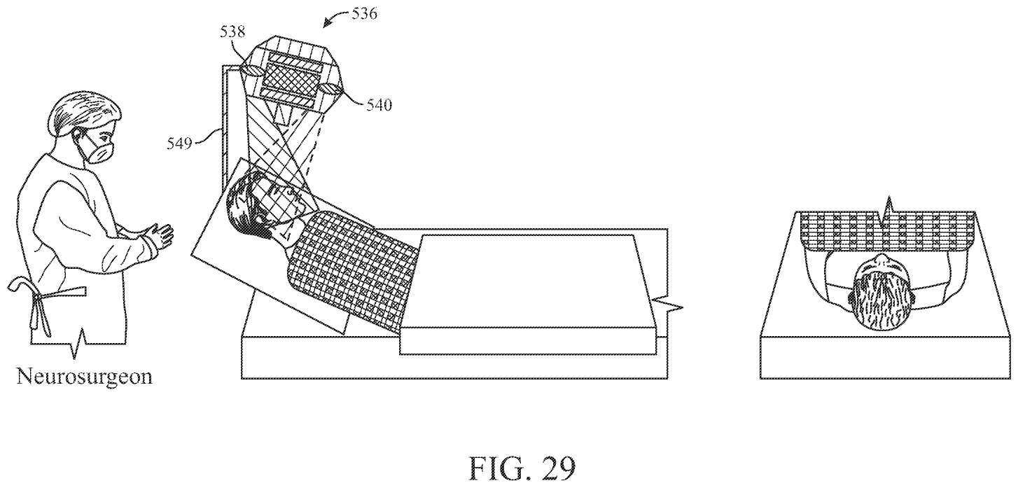

FIG. 29 shows an example of the implementation of step 552. As shown, the guidance system 536 is in the process of acquiring images (using the cameras 538, 540) of the subject's head while in the operating room. The process 550 can be initiated at the bedside of the subject, or while the subject is in the operating room.

Referring back to FIG. 28, once sufficient images are acquired at step 552, the computing device can generate a 3D surface map of the subject's head at step 554. Due to the stereoscopic positioning of the cameras 538, 540, the imaging data (e.g., images) can be used to generate a 3D surface model of the subject's head. The coordinates, shapes, etc., of the 3D surface map can be saved within the computing device. Then, once the 3D surface map is generated at step 554, anatomical features can be extracted at step 556. The anatomical features can be determined based on optical properties of specific features (e.g., being highly reflective). The anatomical features can include, for example, bony structures including but not limited to the nasion, orbits, maxillary prominence, zygomatic arch, external auditory canal of the subject. In some non-limiting examples, the anatomical landmark coordinates can be saved or highlighted within the 3D surface model.

After the anatomical features have been located at step 556, the registration process at step 558 can proceed. A previously generated 3D model of the subject's head (e.g., generated at step 208) is co-registered with the 3D surface map generated from the images. The registration process at step 558 allows the guidance system to track the location and orientation of the user's head in real-time, with a high level of detail (e.g., from the utilization of the 3D model at step 208). Once the registration process at step 558 is completed, a user (e.g., the surgeon) can place an optical marker on the subject at step 560. The optical marker can be a pattern that is easily discernable to the cameras. For example, in some non-limiting examples, the optical marker can be a two-dimensional (“2D”) barcode. The 2D barcode can be sterile, and can have an adhesive backing for adherence to the subject's head. In some cases, the optical marker is preferably placed on the forehead of the subject. Once the optical marker has been placed at step 560, the registration process 558 can occur again. This way, the co-registered 3D model of the head can be tracked live at step 562 relative to the easily discernable optical marker. In some embodiments, such as with usage of other imaging guidance systems (e.g., CT imaging, MRI imaging, or other imaging modalities), the optical marker can rather be an imaging fiducial visible by the corresponding imaging system.

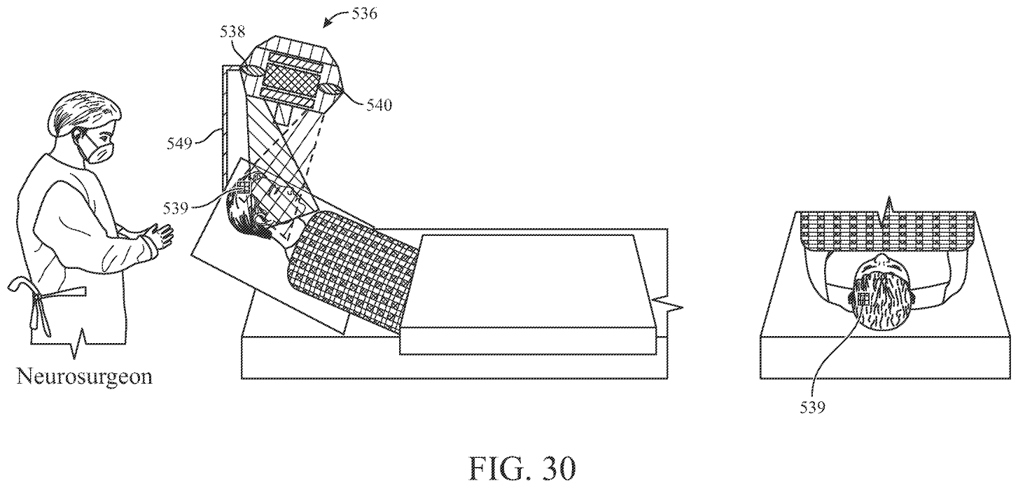

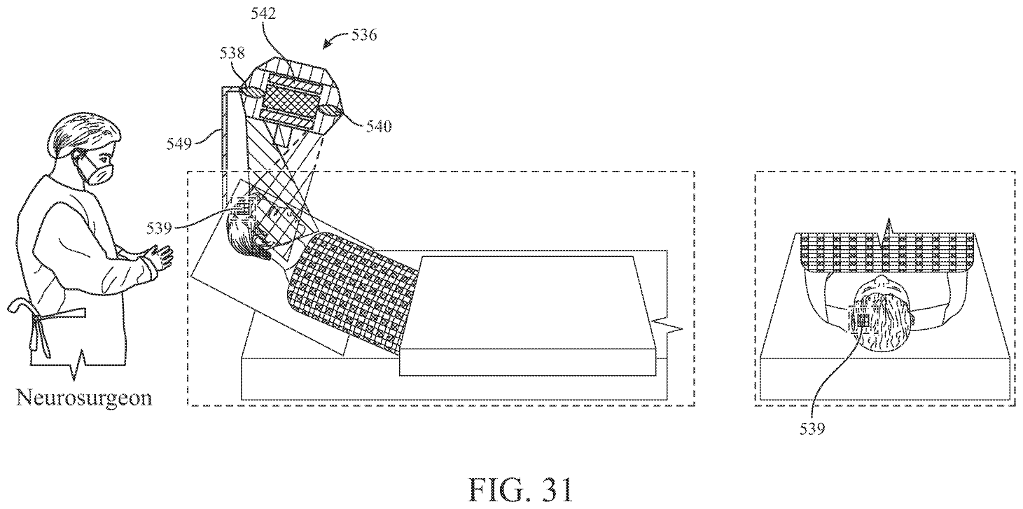

FIG. 30 shows an example of the implementation of steps 558 and 560. As shown, a 2D barcode 539 has been placed on the subject's head. Once the 2D barcode has been placed, the guidance system performs the registration process again. This allows the guidance system to continuously track the location and orientation of the subject's head relative to the guidance system, via the 2D barcode 539. Importantly, once the surgeon drapes the patient (e.g., utilizing a standard of care surgical technique), covering at least some of the optically extractable anatomical features, the guidance system can still track the location and orientation of the patient due to the 2D barcode 539. In some non-limiting examples, allowing the surgeon to drape the patient, allows for absolute sterility, and does not force the surgeon to change their typical practices. The draping of the patient is illustrated in FIG. 31. This ability of the guidance system to track even when the patient is draped, is important because the surgeon does not need to fixate the patients head (e.g., cranial immobilization). In other words, slight movement of the patient's head can be tracked by the guidance system, and thus this procedure can be completed on a subject that is non-immobilized and non-anesthetized.

Referring back to FIG. 28, once the optical tracker has been placed (step 560) and the guidance system has been registered again (step 558), the guidance system can project the determined trajectory at step 564. As discussed above, the trajectory determination system 202 has previously determined the trajectory (step 222). Thus, the coordinates of the trajectory can be transmitted to the guidance system. In some cases, because the trajectory coordinates are in reference to the 3D model generated from imaging data (step 208), the trajectory coordinates can be registered at 558 to the co-registered 3D model. In other cases, registration at step 558 does not manipulate the shape of the 3D model generated from the imaging data. In this scenario, registration at step 558 only moves the 3D model generated from the imaging data in space relative to the tracked optical marker, thus the coordinates of the trajectory do not need to be changed. Regardless of how or if the coordinates of the trajectory have changed (e.g., to adjust to a specific coordinate system), the guidance system can realize the specific trajectory.

In one implementation, the guidance system (e.g., the computing device) can cause the mounting unit to move the orientation and position of the projector, such that it is aligned with the subject at the specific trajectory. In other implementations, the guidance system (e.g., the computing device can move the optical components (e.g., mirrors, lenses, etc.) to adjust the angle the image (or laser) is emitted out relative to the mounting structure. In some cases, an accelerometer can be in communication with the guidance system to provide feedback, with regard to maintaining the specific orientation of the guidance system relative to the subject. Referring back to FIG. 31, the guidance system 536 is implemented to use the projector 542 to emit a light pattern to align with the previously determined trajectory.

In another implementation, the guidance system (e.g., the computing device) includes the augmented reality headset 518. The augmented reality headset 518 can project, to the surgeon, a 3D image scene, which is determined by the guidance system (e.g., the guidance system 536). Then, because the augmented reality headset 518 can be tracked relative to the guidance system 536 (e.g., by coordinate system transformations), the surgeon can augmented view the trajectory. FIG. 32 shows this specific implementation.

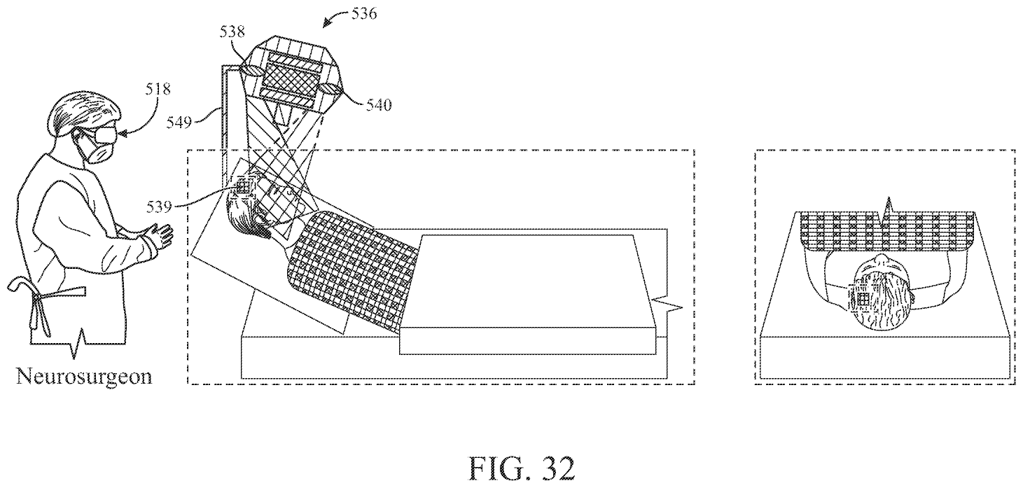

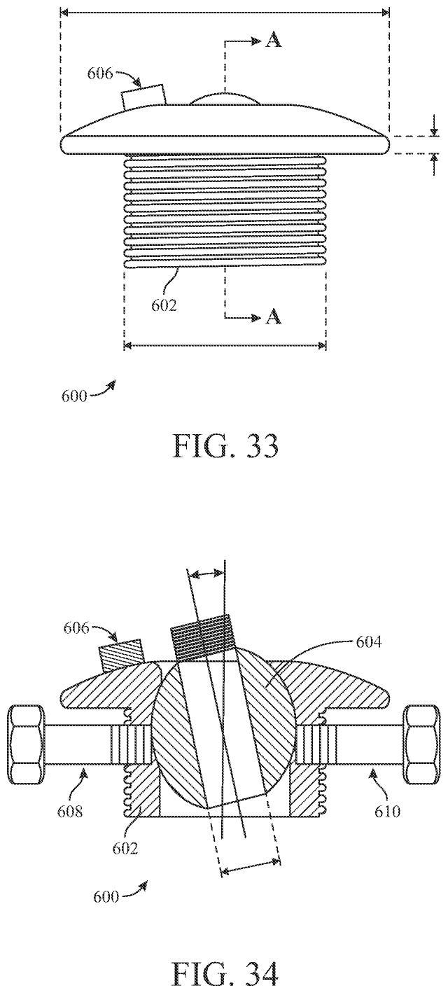

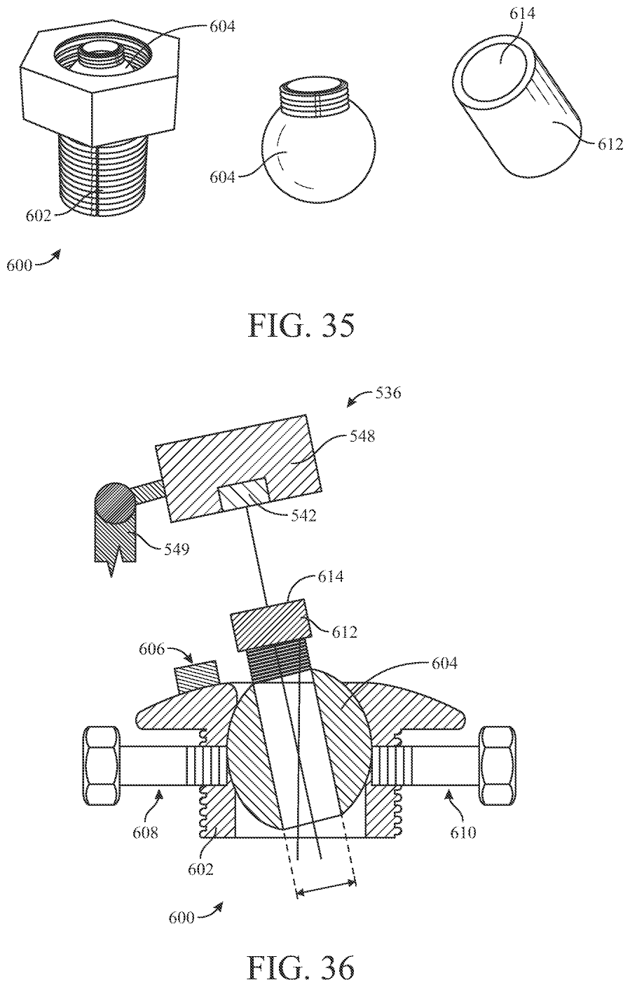

Referring back to FIG. 28, while the determined trajectory is projected (at step 564) the surgeon can install an orient the pivotable cranial screw at step 566. FIGS. 33-35 show an example of a pivotable cranial screw 600. The cranial screw 600 includes a main body 602, a pivotable member 604, locking members 606, 608, 610, and a cap 612. The pivotable member 604 is received within the main body 602, includes a bore, and can adjust its orientation relative to the main body 602. The locking members 606, 608, 610 allow for the pivotable member 604 to be fixed relative to the main body 602. The locking members 606, 608, 610 are illustrated as being fasteners (e.g., bolts) which are advanced (tightened) to abut against the pivotable member 604 to lock the pivotable member 604 in place. In alternative non-limiting examples, however, other locking mechanisms known in the art can be used.

FIG. 36 shows an example of an implementation of step 566. In this implementation the guidance system utilizes a laser (the projector 542) to emit a light pattern at the subject's head. The light pattern aligns with the predetermined trajectory. More specifically, in some cases, if the patient moves their head after the guidance system illuminates the trajectory (and location on the head), the guidance system can move (or move optical components) to ensure that the illuminated trajectory remains aligned with the predetermined trajectory. The light pattern not only shows the orientation, but also the entry location on the head of the subject. Thus, the surgeon should excise the skin and implant the main body 602 into the subject's skull at the illuminated entry location.

Once the main body 602 have been implanted into the skull of the patient at the illuminated target site, the pivotable member 604 can be oriented to align with the trajectory. In some cases, the cap 612 can be threadingly engaged with the pivotal member 604 to help view the illuminated trajectory. More specifically, the cap 612 can have a surface 614 that can help view the illuminated trajectory. For example, when the illumination pattern (trajectory) is centrally located on the cap 612, then the surgeon knows that the orientation of the pivotable member 604 is aligned with the predetermined trajectory. One or more of the locking members 606, 608, 610 can then be advanced to lock the pivotable member 604 in the correct orientation. Once the pivotable member 604 is oriented as desired the cap 612 can be removed. In some cases, the surface 614 can be reflective, such that absence of the illumination pattern on the cap 612 indicates to the surgeon that the pivotable member 604 is in the correct orientation.

In the other implementation where the guidance system includes an augmented reality headset 518, the predetermined trajectory is displayed to the user in a 3D image scene. In some cases, the augmented reality headset 518 can include a glove that is an interfacing component, which can be tracked relative to the subject's head. Thus, while grasping the pivotable cranial screw 600 with the glove, the main body 602 can be placed at the predetermined trajectory, which is displayed to the user. Then, the main body 602 can be implanted at that location (once the location is found the augmented reality headset 518 can be removed to implant the pivotable cranial screw 600). Once implanted, the pivotable member 604 can be grasped with the glove (while the user wears the augmented reality headset 518) and the pivotable member 604 can be oriented to align with the predetermined trajectory displayed to the user. Once sufficiently aligned, one or more of the locking members 606, 608, 610 can be advanced to lock the pivotable member 604 in the correct orientation.

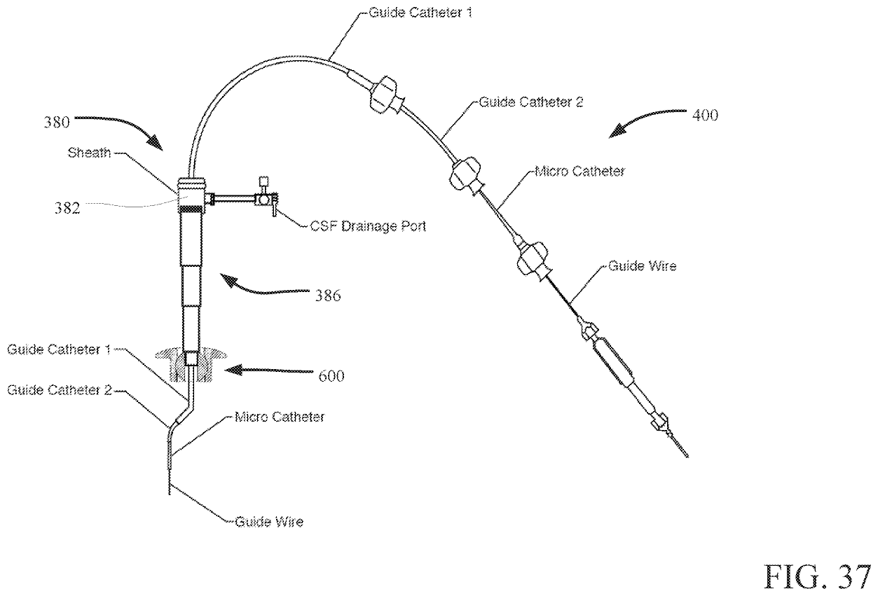

After the pivotable cranial screw 600 has been locked in the desired orientation, the cap 612 can be removed. Then, as shown in FIG. 37, the telescoping adapter 386 can be coupled to the cranial screw 600 and the telescoping adapter 386 can be coupled to the body 382 of the sheath system 380. More specifically, the threads 399 of the telescoping member 386 can be threadingly engaged with the threads 616 to secure the telescoping member 386 to the pivotable cranial screw 600. Additionally, as detailed above, the threads 398 of the telescoping member 386 can threadingly engage the threads of the circular aperture 383 of the body 382. FIG. 37 also shows the surgical system 400 deployed. First, a catheter (e.g., the catheter 381) is inserted through the conduit 388, into the telescoping adapter 386, through the cranial screw 600, and into the ventricle of the subject. Then, after the catheter is secured, other components within the surgical system 400 can be deployed through the catheter (e.g., the catheter 381).

In some non-limiting examples, although the telescoping adapter 386 has been described as being able to axially extend or retract, in some non-limiting examples, the telescoping adapter 386 can be replaced with another adapter. This adapter can be a monolithic piece with a cylindrical bore having two different cross-sections, a first cross section situated at one end of the bore and a second cross-section situated on the opposing end of the bore. The first portion of the bore having the first cross-section can be removably coupled to a surface of the cranial screw (e.g., the cranial screw 364, or the cranial screw 600), and the second portion having the second cross-section can be removably coupled to a surface of a sheath (e.g., the body 382 of the sheath system 380). In another example, this adapter can be a monolithic piece with a bore directed through the entire axial dimension of the adapter. In this case, a first end of the adapter can be removably coupled to a cranial screw, and the second opposing end of the adapter can be removably coupled to the sheath (e.g., the body 382 of the sheath system 380). In particular, for example, a portion of the first end of the adapter can have threads within the bore to threadingly engage a threads of the cranial screw, and a portion of the second end of the adapter can have threads (either in the bore, or on an exterior surface of the adapter) to threadingly engage threads of the sheath (e.g., a protrusion that threadingly engages the bore, or a cavity that threadingly engages the exterior surface of the adapter). In some non-limiting examples, the sheath can be integrally formed with (or coupled, such as not removably coupled) to an adapter.

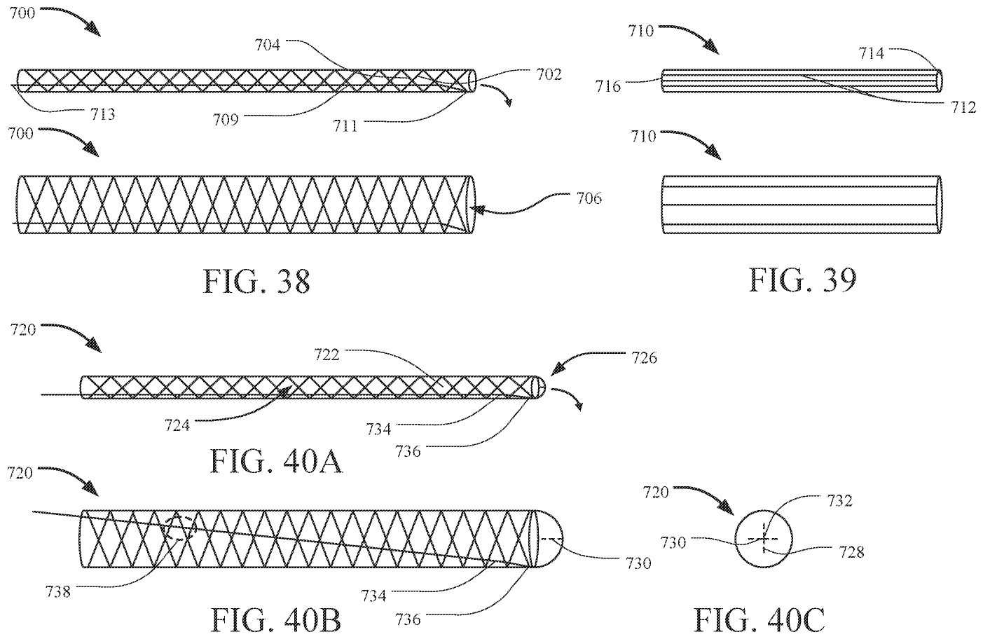

FIG. 38 shows one illustration of a surgical scaffold 700 in a compressed state and another illustration of the surgical scaffold 700 in an expanded state (e.g., after deployment). The surgical scaffold 700 includes support members 702, 704 that are joined together at particular locations, and which are compressed (e.g., folded, bent, etc.) to define the compressed configuration of the surgical scaffold 700. As shown, the support members 702, 704 are joined together to form a tessellated pattern, which is illustrated as being parallelograms. However, in alternative configurations the shapes that form the tessellated pattern can be different than the illustrated parallelograms (e.g., triangles, hexagons, etc.). The support members 702, 704 (and the surgical scaffold 700) are formed out of a resilient material (e.g., spring steel, nickel-titanium alloys, etc.) such that when the surgical scaffold 700 is compressed (e.g., folded) into the compressed configuration, upon removal of the compressive force, the surgical scaffold 700 will spontaneously revert back to its expanded state.

In the illustrated non-limiting example of FIG. 38, the surgical scaffold 700 is shown as being cylindrical in shape, however, in other configurations the overall shape of the surgical scaffold 700 can be different (e.g., a rectangular prism, an octagonal prism, etc.). In some cases, the regions between adjacent support members that define an individual shape of the tessellated pattern can be voids (e.g., apertures). However, in other configurations, these regions can rather include a flexible material (e.g., a flexible plastic, or other biocompatible material), or other folded material (that is biocompatible). In this way, when the support members 702, 704 expand, these regions also expand (e.g., unfold, or the material expanding). Thus, the surgical scaffold 700 can define an internal volume 706 that is fluidically isolated from the exterior environment that surrounds the surgical scaffold 700, which can allow for substantially easy guidance of other instruments through the surgical scaffold 700 (e.g., and prevent instruments from contacting exterior regions, such as non-targeted regions of the brain).

As shown, the upper illustration of the surgical scaffold 700 of FIG. 38 is in a compressed stated, and the lower illustration of the surgical scaffold 700 of FIG. 38 is in an expanded state. The surgical scaffold 700 can be expanded from the compressed state, in a variety of ways. For example, when the surgical scaffold 700 is compressed in the compressed configuration a sheath can be placed around (e.g., coaxially around) the compressed surgical scaffold 700 to maintain the surgical scaffold 700 in the compressed state. Then, as desired, the sheath can be removed (e.g., retracted, cut, etc.) to deploy and expand the surgical scaffold 700 in the expanded state. Although this is one way in which the surgical scaffold 700 can be expanded, other configurations are contemplated. For example, a retaining member (e.g., a wire lock) can be secured around the compressed surgical scaffold 700 to maintain the compressed configuration of the surgical scaffold. Then, as desired, the retaining member can be removed (e.g., unlocked) to allow the compressed surgical scaffold 700 to expand into the expanded configuration.

As shown, the surgical scaffold 700 includes a wire 709 coupled to a distal end of the surgical scaffold 700 at a securement location 711 and extending along the entire axial length of the surgical scaffold 700 and through a proximal end of the surgical scaffold 700. The wire 709 can be implemented in different ways to provide the necessary flexibility and rigidity to manipulate a distal end (or in other words the distal tip) of the surgical scaffold 700. For example, as described below, when tension is provided (at an angle) to the proximal end 713 of the wire that extends out of the proximal end of the surgical scaffold 700, the distal tip of the surgical scaffold 700 can deflect towards the proximal end of the surgical scaffold 700. In particular, the deflection of the distal tip of the surgical scaffold 700 is situated along a plane that is perpendicular to and extends along the axial axis of the surgical scaffold 700 (e.g., where the axial axis is defined along the longitudinal length of the surgical scaffold 700). Thus, the wire 709 can be formed out of flexible yet rigid materials, such as metals (e.g., stainless steel), fabric polymers (e.g., nylon), etc. While the securement location 711 is illustrated as a point on an interior surface of the surgical scaffold 700, in other configurations the securement location 711 can be an area that spans a longitudinal extent of the surgical scaffold 700. Additionally, in some cases, the securement location 711 can be located on an exterior surface of the surgical scaffold 700.

In the illustrated non-limiting example of FIG. 38, the surgical scaffold 700 is expandable for the entire axial length of the surgical scaffold 700. In some cases, the axial length of the surgical scaffold can be the length of a currently available EVD catheter. In some non-limiting examples, the diameter of the surgical scaffold 700 in the compressed state is the diameter of a currently available EVD catheter. For example, the diameter of the surgical scaffold 700 in the compressed state can be about (e.g., deviating by about 20%) 3.5 mm. In some non-limiting examples, the diameter of the surgical scaffold 700 in the expanded state can be about 8-10 mm. In some cases, the ratio of the expanded diameter to the compressed diameter of the surgical scaffold 700 can be in a range from about 2 to 2.5.

FIG. 39 shows one illustration of another surgical scaffold 710 in a compressed state and another illustration of the surgical scaffold 710 in an expanded state (e.g., after deployment). The surgical scaffold 710 functions in a similar manner to the surgical scaffold, but is structured differently. For example, the surgical scaffold 710 includes longitudinal beams 712 that extend along an axial direction and are coupled on one end to a flexible ring 714 and on an opposing end to a flexible ring 716. The longitudinal beams 712 are illustrated as being substantially parallel (e.g., deviating by less than 20%) to the axial axis of the surgical scaffold 710, although other geometric orientations of the beams 712 are possible. The longitudinal beams 712 can be formed out of rigid materials, such as metals, plastics, etc., to maintain the shape of the surgical scaffold 710.

In some cases, such as in the illustrated non-limiting example, the flexible members 714, 716 are ring shaped, while in other cases, the flexible members 714, 716 include a plurality of bends that collectively follow a bulk ring shape. Regardless, the flexible members 714, 716 retract in the compressed state, and expand from the compressed state, into the expanded state. The flexible members 714, 716 can be formed of resilient (e.g., spring-like) material that can be compressed into a compressed state by a compressive force, and can spontaneously expand into the expanded state upon removal of the compressive force. Although the flexible members 714, 716 are illustrated as being positioned on opposing ends of the surgical scaffold 710, in other configurations, there can be more or fewer flexible members located at similar or different positions. For example, in some configurations, the surgical scaffold 710 can include a single centrally located (e.g., central relative to the axial length) flexible member.

Similarly to the surgical scaffold 700, the surgical scaffold 710 can be expanded from the compressed state and to the expanded state in a number of different ways. For example, a sheath can be placed coaxially over the compressed surgical scaffold 710 to maintain the compressed state of the surgical scaffold 710. Then, as desired, the sheath can be removed to selectively expand the compressed surgical scaffold 710 into an expanded surgical scaffold 710. In particular, the flexible members 714, 716 are allowed to expand, which also expands the spacing between adjacent beams 712. In some non-limiting examples, and similarly to the surgical scaffold 700, the regions between adjacent beams 712 can either be voids (e.g., apertures), or can be flexible or folded materials, either of which can selectively expand and retract along with the surgical scaffold 710. Similarly to surgical scaffold 700, the surgical scaffold 710 can also include a wire (not shown).

In the illustrated non-limiting example of FIG. 39, the surgical scaffold 710 is expandable for the entire axial length of the surgical scaffold 710. In some cases, the axial length of the surgical scaffold can be the length of a currently available EVD catheter. In some non-limiting examples, the diameter of the surgical scaffold 710 in the compressed state is the diameter of a currently available EVD catheter. For example, the diameter of the surgical scaffold 700 in the compressed state can be about (e.g., deviating by about 20%) 3.5 mm. In some non-limiting examples, the diameter of the surgical scaffold 710 in the expanded state can be about 8-10 mm. In some cases, the ratio of the expanded diameter to the compressed diameter of the surgical scaffold 710 can be in a range from about 2 to 2.5.

In some non-limiting examples, the surgical scaffolds 700, 710 can form part of a catheter, and in particular a ventriculostomy catheter. For example, either of the surgical scaffolds 700, 710 can interface with a catheter. As a specific example, either of the surgical scaffolds 700, 710 can be coupled to an interior surface of a catheter, can be coupled to an exterior surface of a catheter, and can be interfaced within a wall of a catheter (e.g., one layer of the catheter and another layer of the catheter are sandwiched together to encapsulate a scaffold).

FIG. 40A-40C shows an example of an expandable catheter 720 in the compressed (or contracted) state (FIG. 40A), and in the expanded state (FIGS. 40C, 40C). The expandable catheter 720 includes tube 722, and an expandable scaffold 724. The tube 722 generally defines the interior volume of the catheter, and can be formed of different biocompatible materials. The expandable scaffold 724 can be coupled to the interior surface of the tube 722, the exterior surface of the tube 722, or integrated within the wall of the tube 722. The expandable scaffold 724 can be implemented in different ways, and can be the surgical scaffolds 700, 710. For example, in the illustrated non-limiting example, the expandable scaffold 724 is implemented as the surgical scaffold 700 and is coupled to the exterior surface of the tube 722.

As shown, the expandable catheter 720 includes a tip portion 726 coupled to a distal end of the tube 722. The tip portion 726 can be formed of the same material as the tube 722, or can be formed of other flexible materials. The tip portion 726 can include two slits 728, 730 that overlap each other at a central location 732. Although two slits 728, 730 are illustrated, in other configurations, additional numbers (or fewer numbers) of slits can be utilized. For example, four slits that overlap at the central location 732 could be utilized on the tip portion 726, which may allow for easier advancement of instruments through the expandable catheter 720. The flaps that are defined between adjacent sides of a portion of a slit can be moved (or retracted) so as to allow for the advancement of an instrument through the distal end of the expandable catheter 724. In this way, the slits 728, 730 both allow for the advancement of instruments, while providing a reasonable seal when instruments are removed.

In some non-limiting examples, the diameter of the expandable catheter 720 in the compressed state is the diameter of a currently available EVD catheter. For example, the diameter of the expandable catheter 720 in the compressed state can be about (e.g., deviating by about 20%) 3.5 mm. In some non-limiting examples, the diameter of the expandable catheter 720 in the expanded state can be about 8-10 mm. In some cases, the ratio of the expanded diameter to the compressed diameter of the expandable catheter 720 can be in a range from about 2 to 2.5. In some embodiments, the expandable catheter 720 can include holes similar to those of the catheter 381, which can be located on a distal end of the expandable catheter 720. In some cases, these holes can facilitate the drainage of CSF fluid from the ventricle.

Similarly to surgical scaffolds 700, 710, the expandable catheter 720 can also include a wire 734 that is coupled to a portion of a distal end of the expandable catheter 720 at a securement location 736. The wire 734 extends along the entire axial extent of the expandable catheter 720 and further extends out of a proximal end of the expandable catheter 720. Although the expandable catheter 720 is shown being coupled to an interior surface of the expandable catheter 720, in other cases, the expandable catheter 720 can be coupled to an exterior surface of the expandable catheter, or sandwiched between an exterior and interior surface of the expandable catheter 720. In some cases, the wire 734 can be received through a sleeve (not shown) coupled to an exterior surface of the expandable catheter 720. In this way, the wire 734 can be received through the sleeve and extend past the proximal end of the expandable catheter 724, such that the wire 734 itself does not undesirably contact instruments received through the interior volume defined by the expandable catheter 720. In some cases, rather than the wire 734 extending through the proximal end of the expandable catheter 720, the wire 734 can be introduced through a hole 738 of the expandable catheter 720 at a predetermined location relative to the tip portion 726 (e.g., or the distal end) of the expandable catheter 720. In this way, the wire 734 can be manipulated more easily.

Similarly to the surgical scaffolds 700, 710, the expandable catheter 720 can also be expanded from the contracted (or compressed) configuration in a number of ways. For example, a sheath can be coaxially placed over the expandable catheter 720 while the expandable catheter is in the compressed configuration. Then, as desired, the sheath can be removed to selectively expand the expandable catheter 720 from the compressed configuration and into the expanded configuration. In particular, the expandable scaffold 724 of the expandable catheter 720 expands the tube 722 (and in some cases the tip portion 726) by either expanding the walls of the tube 722 or unfolding the walls of the tube 722. Similarly, the tip portion 726 can also be expanded by expanding the walls of the tip portion 726 or unfolding the walls of the tip portion 726 (via the expandable scaffold 724). Once expanded, the internal volume of the expandable catheter (or other expandable structure) defines a surgical corridor, which can facilitate the relatively easy insertion of instruments to the target region through the expandable catheter. Similarly, the tube (and in some cases the tip portion) can substantially block undesirable fluids exterior to the expandable catheter from entering the interior volume of the catheter, and can guide instruments to the target location. In some non-limiting examples, the circumferential expansion (or radial expansion) of the surgical scaffolds and the expandable catheter can cause atraumatic dissection of the tissue (e.g., of the brain parenchyma). In other words, when the tissue retracts from removal of the surgical scaffolds or the expandable catheter, the tissue that was dissected is not harmed.

In some non-limiting examples, the surgical scaffolds 700, 710, and the expandable catheter 720 (e.g., surgical scaffold 724). the can be formed out of selectively retractable and expandable materials, based on the temperature of the expandable material. For example, components of the surgical scaffolds 700, 710, and the expandable catheter 720 that provide rigidity to these (e.g., the support members) can be formed out of super elastic materials such as nitinol that has a transition temperature near the temperature of the human body (e.g., 37° C.). In this way, below the transition temperature the scaffolds and expandable catheter can be configured in the compressed state, and after a certain time such that the super elastic materials reach or exceed the transition temperature, the surgical scaffold and expandable catheter can spontaneously expand to the expanded configuration. In some cases, a cooling system or heating system can be placed in thermal communication with these components so as to selectively expand or retract these devices. In some cases, the heating or cooling systems can be controllable by a computing device.

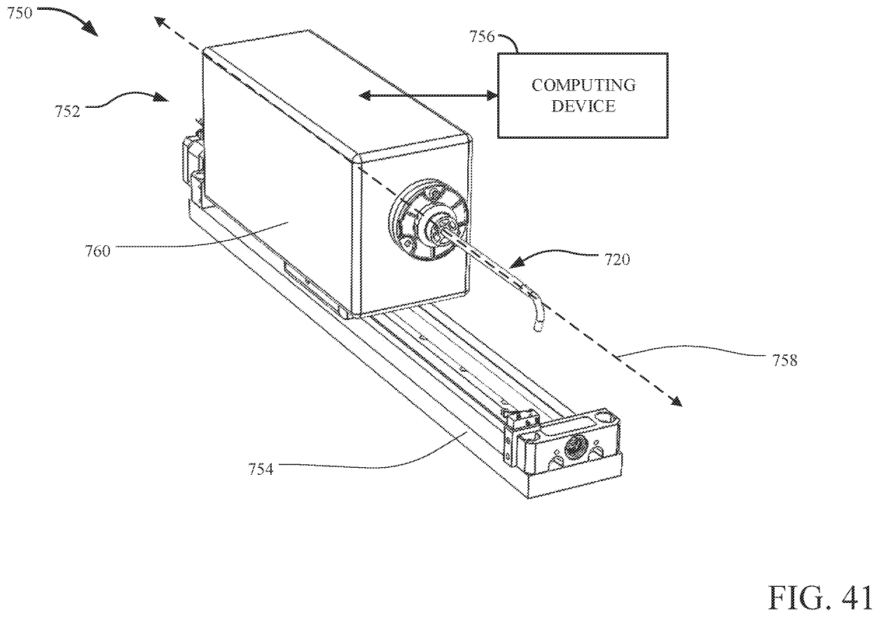

FIG. 41 shows an isometric view of an example of a robotic system 750 for deployment of a surgical scaffold for ventricle procedures (e.g., ventriculostomy procedures). The robotic system 750 includes a robotic assembly 752, a rail 754, and a computing device 756. The robotic assembly 752 is configured to translate along the rail 754 so as to bring the robotic assembly 752 closer (or farther) from a target location on a subject. In some cases, the rail 754 (or a component of the robotic assembly 752 that engages with the rail 754) can include a locking device (e.g., a brake) that is actuatable by the computing device 756. In this way, after the desired position has been reached, the computing device 756 can actuate the locking device (such as an actuator) to lock the position of the robotic assembly 752 along the rail 754. The computing device 756 is in communication with the robotic assembly 752 and other computing devices. The computing device 756 can be implemented in similar ways as the other computing devices (or systems) described above. In some non-limiting examples, the computing device 756 can relate the coordinate system of the robotic assembly 752 with an imaging system that images a subject. In this way, the positioning of the components of the robotic system 750 can be related to the position of the subject, and in particular a located target point on the subject.

As shown, the expandable catheter 720 with a coaxial sheath (not shown) disposed coaxially over the expandable catheter 720 is interfaced with the robotic system 750 to be deployed. In particular, the expandable catheter 720 partially extends along an axial axis 758 of a housing 760 of the robotic assembly 752. As described below, the robotic assembly 752 is configured to orient the distal tip 726 of the expandable catheter 720 along a predetermined 3D line and with the distal tip 726 oriented along the predetermined 3D line, advance the expandable catheter 720 (and coaxial sheath) along the 3D line. Although the robotic system 750 is described with respect to the expandable catheter 720, in other configurations the robotic system 750 can deploy other surgical scaffolds, catheters, guidewires, etc.

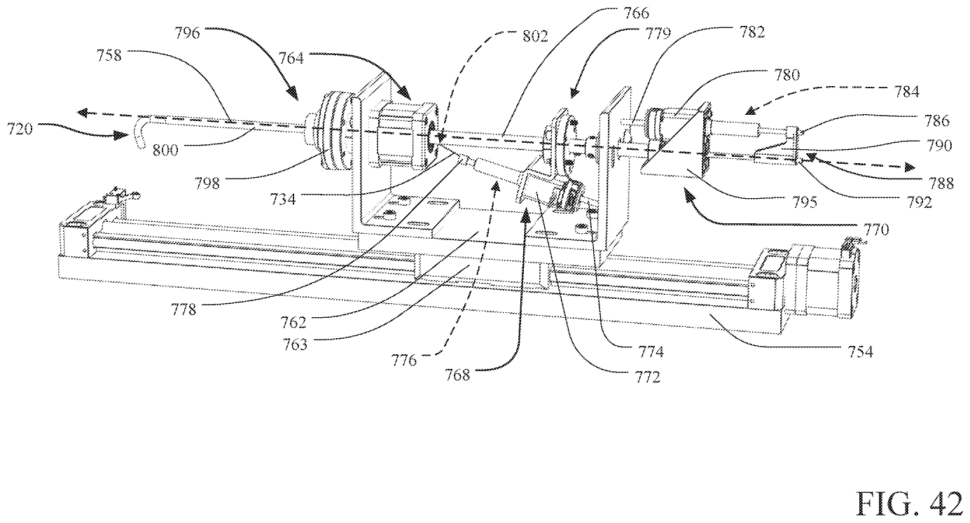

FIG. 42 shows another isometric view of the robotic system 750 with a portion of the housing 760 of the robotic assembly 752 removed for visual clarity. The housing 760 includes an outer shell (removed from the view of FIG. 42) and a support 762 that secures the components within the robotic assembly 752. The support 762 also includes an extension 763 that downwardly extends from the support 762 and includes a slot that surrounds and engages with the rail 754 to translate the support 762 along the rail 754. The robotic assembly 752 also includes a motor 764, a rotatable shaft 766 that has a bore therethrough, a linear actuator 768, and an advancer 770. In some non-limiting examples, the motor 764 is configured to rotate the rotatable shaft 766 about the axial axis 758 of the housing 760. For example, in some cases, the rotatable shaft 766 can threadingly engage a gear on the motor 764, which thereby rotates the rotatable shaft 766 about the axial axis 758 (e.g., when the gear rotates). As shown, the rotatable shaft 766 extends through the proximal end of the support 762 along the axial axis 758 and engages with the advancer 770. The distal end of the rotatable shaft 766 engages with the motor 764 that is mounted to the distal end of the support 762. In some configurations, the motor 764 is an electric motor and is actuatable by the computing device 756 (or others). In some cases, the rotatable shaft 766 or the motor 764 can have rotational sensors (e.g., encoders) so that the rotational position of the rotatable shaft 766 can be sensed and transmitted to the computing device 756.

The linear actuator 768 includes a motor 772, a lead screw 774, and a nut 776 coupled to a shaft 778. The nut 776 is blocked from translating (e.g., by being contacting by the housing of the linear actuator 768) and threadingly engages with the lead screw 774. As shown, an end of the wire 734 is coupled to the shaft 778, which is angled relative to the axial axis 758. Thus, when activated, the motor 772 rotates the lead screw 774 thereby translating the nut 776 and the shaft and thereby applying tension to the wire 734. In some configurations, the linear actuator 768 can be replaced with the motor 772 itself. In this way, the motor 772 when activated, rotates a gear (or a gear) that is engaged with the wire 734 so that the wire 734 can be wound around the gear (or the shaft) and thereby generating tension in the wire 734. Activation of the motor 772 in the opposing direction (in either configuration) can remove tension from the wire 734 by retreating the wire 734 back towards the distal end of the expandable catheter 720.

As shown, the linear actuator 768 is coupled to the shaft 766 via an extension 779. The extension 779 includes a bent flange that is coupled to the and extends from the shaft 766, which secures the motor 772. Thus, as the extension 779 rotates, the linear actuator 768 also rotates. In this way, as described below, the deflected tip of the expandable catheter 720 can be rotated around the axial axis 758, allowing the deflected tip of the expandable catheter 720 to align with nearly all 3D lines (e.g., nearly any predetermined trajectory that is a 3D line).

The advancer 770 is configured to translate a portion of the expandable catheter 720 (including the coaxial sheath) along the axial axis 758. In some configurations, such as in the illustrated non-limiting example, the advancer 770 can be implemented as a linear actuator that includes a motor 780, a lead screw 782, a nut 784 coupled to a shaft 786, and a plunger assembly 788. The plunger assembly 788 includes a support 790, and a shaft 792 that extends along the axial axis 758. The support 790 couples together the shafts 786, 792 so that they translate together. Similarly to the linear actuator 768, the nut 784 of the advancer 770 implemented as the linear actuator is blocked from rotating and thus translates along the lead screw 782 which extends in a direction that is parallel (and offset) to the axial axis 758. The advancer 770 also includes an engager 794 that releasably secures an end of the expandable catheter 720 (or other instrument, such as a surgical scaffold as described above) at one end and which is coupled to the shaft 792 on the other end. Thus, activation of the motor 780 (e.g., via the computing device 756) translates the plunger assembly 788 thereby advancing the expandable catheter 720 along the axial axis 758.

As shown, the advancer 770 is positioned rewardly along the axial axis 758 and relative to the motor 764, the linear actuator 768, and the expandable catheter 720. In some non-limiting examples, the advancer 770 also includes a housing 795 that is secures the appropriate components of the advancer 770 to the housing 760 (e.g., the motor 780). All the motors 764, 772, 780 can be controllable by the computing device 756 and are all configured to rotate in two rotational directions relative to their respective rotational axis. In other words, the motors 764, 772, 780, can rotate forwards and backwards. In some cases, similarly to the motor 764, the motors 772, 780 can include rotational sensors (or position sensors for the shafts) to determine a relative position of the respective shafts. In this way, the computing device 756 can determine how far the expandable catheter 720 has been advanced along the axial axis 758 (e.g., via the position of one of the shafts 786, 792) and how much tension has been applied to the wire 734 (e.g., via the position of the shaft 778). In some non-limiting examples, the motors 764, 772, 780 can be implemented as electric motors.

The robotic assembly 752 also includes a fixed head 796 that is coupled to the housing 760, and more specifically, the distal end of the support 762. The fixed head 796 includes a disc assembly 798 having a bore therethrough, and a shaft 800 having a bore therethrough coupled to the disc assembly 798. In particular, one end of the disc assembly 798 is coupled to the support 762 at one end and coupled to the shaft 800 at another end. Thus, the shaft 800 is fixed relative to the support 762, which provides a fixed point for the distal tip of the expandable catheter 720 to be deflected about. In some non-limiting examples, respective bores are located through respective ends of the support 762 along the axial axis 758. This allows the expandable catheter 720 to extend through the bore of the shaft 766, through the distal side of the support 762, and through the fixed head 796 (e.g., through the bore of the disc assembly 798 and through the bore of the shaft 800).

As shown, the shaft 766 includes a hole 802 that is directed through the entire thickness of the shaft 766. In this way, and as illustrated, the wire 734 can be threaded through the hole 802 and secured to the linear actuator 768 to provide tension to the wire 734.

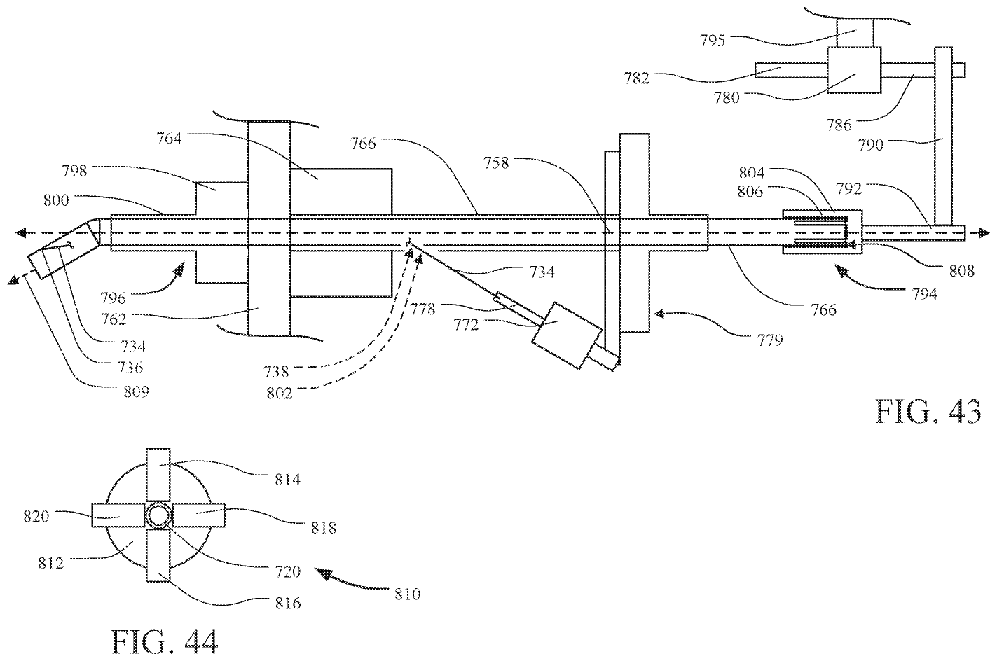

FIG. 43 shows a schematic illustration of a simplified cross-sectional view of the robotic system 750 taken along the axial axis 758 of FIG. 42. As shown, the engager 794 includes cylinders 804, 806 that are positioned coaxially relative to one another and relative to the axial axis 758. In particular, the cylinder 806 having a smaller dimeter than the cylinder 804 is nested within a bore of the cylinder 804 and is coupled to the cylinder 804. A slot 808 is radially situated between the cylinders 804, 806 and receives the proximal end of the expandable catheter 720 (e.g., with the coaxial sheath). In particular, the proximal end of the expandable catheter 720 is slid onto the cylinder 806 and is thus received within the slot 808. In some cases, the slot 808 is sides so that the cylinder 806 contacts the interior surface of the expandable catheter 720 while the cylinder 804 contacts the exterior surface of the expandable catheter 720. In some non-limiting examples, the diameter of the cylinder 806 is slightly lager (e.g., 5% greater than) the diameter of the expandable catheter 720 so that when the proximal end of the expandable catheter 720 is slid onto the cylinder 806, the proximal end of the expandable catheter 720 is slightly stretched and retracts radially around the cylinder 806 thereby providing a tight interface relative to the engager 794.

In some non-limiting examples, the cylinder 804 can be removed where the engager 794 only includes the cylinder 806, which may make installing of the expandable catheter 720 easier. In this case, for example, the cylinder 806 can take other shapes that substantially corresponds to the shape of the inner surface of the proximal end of the expandable catheter 720, such as prisms (e.g., octagonal prisms, hexagonal prisms, etc.). Additionally, although the engager 794 has been described with cylinders 804, 806, in other non-limiting examples, the cylinders 804, 806 can have other shapes (e.g., prims), but where the slot 808 has a shape that substantially corresponds to the shape of the inner surface of the proximal end of the expandable catheter 720.

As shown, the proximal end of the wire 734 has been received through the hole 738 of the expandable catheter 720 and through the hole 802 of the shaft 766, which is secured to the shaft 778. In the illustrated non-limiting example of FIG. 43, the expandable catheter 720 extends from its proximal end that is engaged with the engager 794 through the bore of the shaft 766 and through the bores of the head assembly 796 until the distal end of the expandable catheter 720 extends out of the bore of the shaft 800 a predetermined amount. The motor 772 is activated (e.g., by the computing device 756) and retracts the shaft 778 a particular amount thereby applying an amount of tension to the wire 734 that corresponds to the particular amount of retraction of the shaft 778. As tension is applied to the wire 734, the wire 734 pulls and thereby deflects the distal end of the expandable catheter 720. In particular, one side of the expandable catheter 720 abuts against the surface of the shaft 800, and the opposing side of the catheter 720 is pulled via the securement location 73. Because these are separated by a distance that is perpendicular to the axial axis 758 and extends along the plane defined by the view in FIG. 42, the distal end of the expandable catheter 720 deflects towards the housing 460. As such, varying the tension in the wire 734 varies the amount of deflection of the distal end of the expandable catheter 720, which corresponds to the amount of extension of the shaft 778 (e.g., and corresponding the amount of rotation of the motor 772). Thus, the computing device 456 can cause the motor 772 to rotate a specific amount, which causes the distal end of the expandable catheter 720 to deflect a corresponding amount. In this way, the computing device 456 having received a desired trajectory (e.g., an ideal trajectory) that is defined by a 3D line, can cause the motor 772 to deflect the distal end of the expandable catheter 720 a desired amount that corresponds to the 3D line.

As shown in FIG. 43, the motor 764 is engaged with the shaft 766 an is configured to rotate the shaft 766, and thereby rotate the expandable catheter 720 that is situated within the bore of the shaft 766. In some non-limiting examples, the computing device 456 can rotate the shaft 766 to a position that corresponds to the 3D line. In particular, for example, with the distal end of the expandable catheter 720 deflected, the shaft 766 can be rotated thereby rotating the expandable catheter 720 until the deflected distal end of the expandable catheter 720 aligns with the 3D line. In some cases, the computing device 456 can rotate the shaft 766 prior to the deflection of the distal end of the expandable catheter 720. Thus, the motors 764, 772 are configured to collectively align the distal end of the expandable catheter 720 along the 3D line.

In some non-limiting examples, once the deflected distal end of the expandable catheter 720 is aligned with the 3D line that was received (or retrieved) by the computing device 456, the expandable catheter 720 can be extended. For example, the computing device 756 can cause the motor 780 to advance the proximal end of the expandable catheter 720 along the axial axis 758. As the motor 780 advances the proximal end of the expandable catheter 720 and with the tension continually applied by the motor 772, the distal end of the catheter 720 advances along a 3D line 809 defined by the previous deflection of the distal end of the catheter 720.

FIG. 44 shows a schematic illustration of another advancer 810 that can be replaced with the advancer 770 to advance the expandable catheter 720. The advancer 810 includes a support 812 and wheels 814, 816, 818, 820 coupled thereto. The wheels 814, 816, 818, 820 are mounted to surround a bore through the support 812 in which the expandable catheter 720 is received through. In particular, the wheels 814, 816, 818, 820 are mounted on the support 812 at substantial right angles to each other (e.g., deviating by less than 5%). For example, the wheels 814, 816 are positioned on opposing sides of the bore of the support 812, and the wheels 818, 820 are positioned on opposing sides of the bore of the support 812. The wheels 814, 816, 818, 820 are rotatable by respective motors (not shown), that are in communication with and controllable by the computing device 756. The wheels 814, 816, 818, 820 can be rotated to advance or retract the expandable catheter 720. For example, each wheel 814, 816, 818, 820 contacts a corresponding circumferential extent of the expandable catheter 720 to extend or retract the expandable catheter 720. In some cases, the wheels 814, 816 can be rotated in opposing rotational directions and the wheels 818, 820 can be also rotated in opposing rotational directions that can collectively advance the expandable catheter 720 (e.g., along the axial axis 758) or retract the expandable catheter 720 (e.g., if all the motors rotational directions are revered). In some non-limiting examples, these motors can also be electrical motors.

In some non-limiting examples, the advancer 810 can be positioned at a similar location as the advancer 770. In other configurations, the advancer 810 can be positioned exterior to the housing 760 (and support 762). For example, the advancer 810 can be mounted to the shaft 800. In some cases, slots can be directed through the shaft 800 (e.g., if the advancer 810 is not positioned near the end of the shaft 800) so that each of the wheels 814, 816, 818, 820 can still contact the respective circumferential extent of the expandable catheter 720. Although the advancer 810 has been described with the expandable catheter 720, in other configurations, the advancer 810 can advance other instruments (e.g., surgical scaffolds, guidewires, etc.).

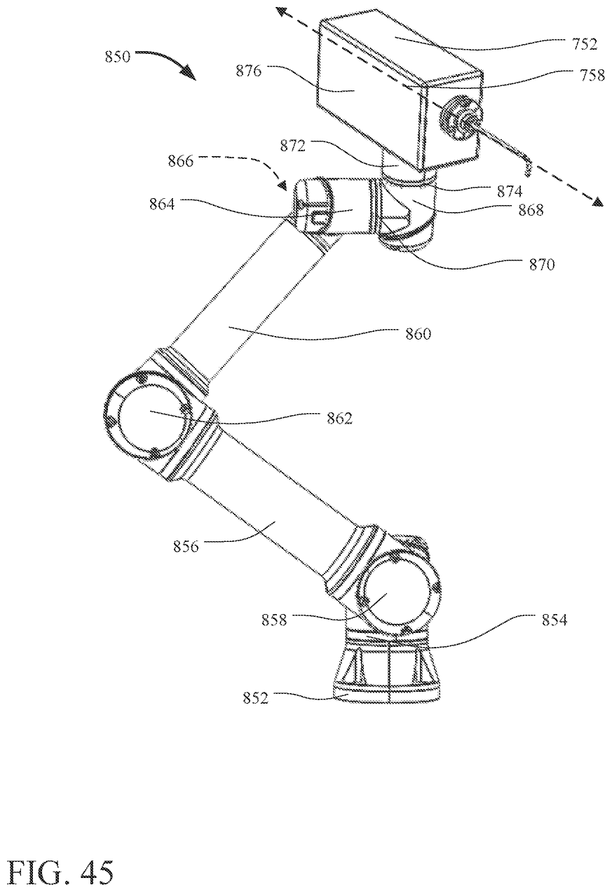

FIG. 45 shows a schematic illustration of a robot arm 850. The robot arm 850 is implemented as a multi-axis robot having at least three degrees of freedom and an end effector. However, in the illustrated non-limiting example the robot arm 850 is a six axis robot arm having six degrees of freedom. For example, the robot arm 850 includes a base 852, a rotatable axis 854, an arm 856 coupled at a rotatable axis 858, an arm 860 coupled at a rotatable axis 862, an arm 864 coupled at a rotatable axis 866, an arm 868 coupled at a rotatable axis 870, and an arm 872 coupled at a rotatable axis 874 at one end and an end effector 876 coupled at an opposing end. In this configuration, the end effector 876 of the robotic arm 850 is the robotic assembly 752. Each axis 854, 858, 862, 866, 870, 874 includes motors (not shown), and positional sensors (not shown) such as toque sensors that can determine the current position of each of the axes axis 854, 858, 862, 866, 870, 874. The robotic arm 850 is in communication and controllable by a computing device (e.g., the computing device 756), and thus the computing device can determine the current location of the end effector of the robot arm 750 and cause the robot arm 750 to move to a particular location (e.g., the entry point of a subject). In some cases, the coordinate system of the robot arm 850 can be related to the coordinate system of the robotic assembly 750. Additionally, the coordinate system of the robot arm 850 and the coordinate system of the robotic assembly 750 can be related to the coordinate system of an imaging system. Thus, the computing device can cause the end effector 876 of the robot arm 850 to move to a particular location as sensed by the imaging system (e.g., a desired target location on a subject), and move the robot arm to adjust the orientation of the end effector 876 along a desired predetermined orientation.

In some non-limiting examples, the computing device can receive a desired 3D line that intersects the target location on the subject (e.g., the final trajectory from a trajectory determining system). Then, the computing device can cause the robot arm 850 to move the end effector 876, which is the robotic assembly 752, and in particular the shaft 800 of the fixed head 796 so that the shaft 800 aligns with the 3D line and an end of the shaft 800 is situated near the target location on the subject. In some cases, computing device 756 can cause the robot arm 850 to advance the shaft 800 until the shaft 800 extends slightly past the target location but remains aligned with the 3D line. Then, the computing device can cause the advancer 770 (or the advancer 810) to advance the expandable catheter 720 along the 3D line until the expandable catheter 720 has been extended a predetermined amount (e.g., as sensed by the motor 780), or until the expandable catheter 720 has been extended a desired amount as determined by an imaging system. In some non-limiting examples, the end effector 876 can simply be an actuator (in communication with the computing device) and having a housing that defines an axial axis (e.g., similar to the axial axis 758). In this way, the actuator engages with and can extend and retreat the expandable catheter 720 (or other instrument) along the axial axis of the housing.

In some non-limiting examples, the robot arm 850 having additional rotational axes is desirable at least because the robot arm 850 can realize more positions than a robot arm having a lower number of rotational axes. In some cases, because the coordinate systems are registered between an imaging system and the robot arm 850, the robot arm 850 can compensate for patient movement (e.g., that is detected by the imaging system). In this way, the head of the subject is not necessarily required to be secured relative to a support structure (e.g., a patient bed, an operating table, etc.).

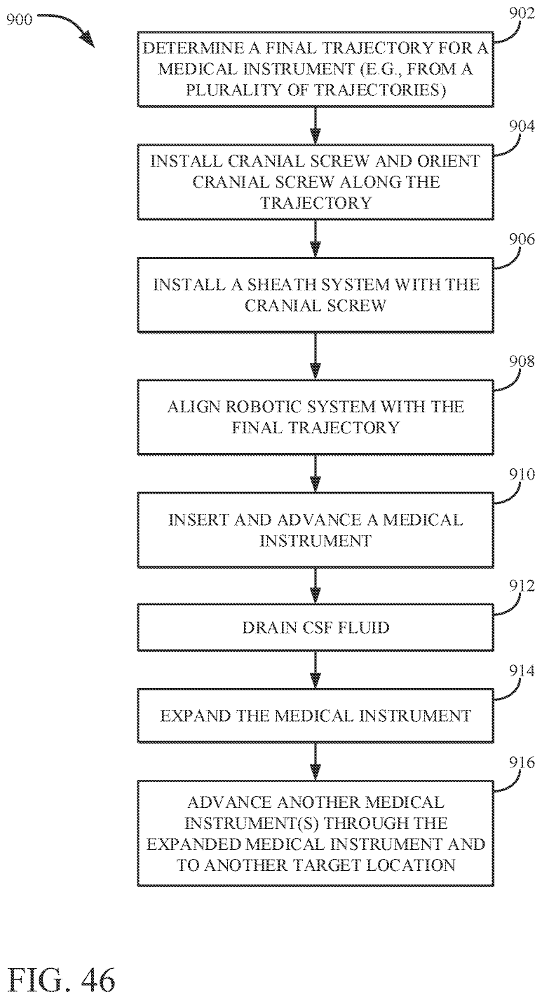

FIG. 46 shows an example of a flowchart of a process 900 for implementing a neurosurgical procedure, parts or all of which (as appropriate) can be implemented on one or more computing devices (e.g., the computing device 756).

At 902, process 900 can include a computing device (e.g., the computing device 756) determining a final trajectory for a medical instrument (e.g., an expandable catheter, a surgical scaffold, etc.). In some cases, this can include a computing device implementing all (or some) of the (appropriate) steps of the determine trajectory process 204 of FIGS. 2 and 3. For example, the computing device can select a final trajectory from a plurality of trajectories that do not intersect any vascular structure of a 3D vasculature model of the subject. In some non-limiting examples, the final trajectory is a 3D line with a target location. In particular, the 3D line can be defined between the target location on the head of a patient (e.g., the skull) and an end point within the desired ventricle of the subject. In some cases, the coordinates that define the 3D line and the slope of the 3D line can be stored in memory (of the computing device) or can be transmitted to a different computing device (e.g., a server).

At 904, process 900 can include a computing device aiding with installing a cranial screw and orienting the cranial screw along the trajectory. In some cases, this can include a computing device implementing some (or all) of the (appropriate) steps of the processes for guiding the installment of the cranial screw via the guidance system 206. For example, this can include the computing device implementing the appropriate steps for creating the 3D custom mask. For example, the computing device can generate a 3D model of a mask of a subject that includes an anatomical feature that corresponds to the head of the subject (e.g., the mask contouring to the shape of the nose of the subject, to the shape of a brow ridge of a subject, to the shape of the curvature of a portion of a head of the subject, etc.), and a bore that intersects with the final trajectory being the 3D line of the subject (e.g., in some cases the 3D line or an extension along the 3D line intersects the center of the bore of the 3D mask). In some configurations, a computing device can 3D print the 3D model of the custom mask of the subject (e.g., via the 3D printing system 308), or form the custom mask from the 3D model (e.g., via a milling machine, such as a computer numeric control mill).

In some non-limiting examples, after the custom mask is formed, a practitioner can place the custom mask on the subject. In some cases, a mask attachment (e.g., the mask attachment 328) can be fitted on the custom mask, which also has a bore that aligns with the bore of the custom mask. Once the mask (and other components of the mask) have been installed on the subject (e.g., the anatomical features of the custom mask interfacing with the corresponding anatomical features of the subject's head), the skin of the scalp contained within the bore of the custom mask can be resected. Then, a drill can drill a hole through the skull at a location within the bore of the custom mask (e.g., center of the bore). Once a hole has been drilled through the skull at this location, a cranial screw (e.g., the cranial screw 364) can be engaged with the hole and installed accordingly.

As another example, the processes for guiding the instalment of a cranial screw via the guidance system can include a computing device implementing some (or all) appropriate steps of the process 550. For example, the computing device can cause a projector (or a laser) to project (or emit) an illumination pattern on the target location on the head of the subject. The illumination of the pattern (e.g., an illuminated circle) can indicate to the practitioner the location of the scalp to resect. For example, while the illumination pattern illuminates the target location on the head of the subject, the practitioner can shave the hair of the subject (as needed), and create cuts through skin that start at the scalp and radially emanate away from the target location. Then, again with the illumination pattern illuminated, the practitioner can drill (using a drill) a hole through the skull normal to the illuminated target point. In some cases, the practitioner can even drill (using a drill) a hole that is oriented along the illuminated pattern, as the computing device can cause the illuminated pattern to extend along the 3D line.

In some non-limiting examples, once the hole is created through the skill, a cranial screw (e.g., the main body 602 of the pivotable cranial screw 600) can be inserted into and engaged with the walls of the skull that define the hole. Then, in some cases, such as when the cranial screw is implemented as a pivotable cranial screw, with the illumination pattern illuminating along the 3D line, the pivotable member of the cranial screw can be oriented until the pivotable member aligns with the illumination pattern. Then, the pivotable member can be locked (e.g., by a user advancing a fastener to contact and thereby lock the pivotable member).

At 906, process 900 can include installing a sheath system with the cranial screw. In some cases, this can include coupling an adapter (e.g., the telescopic adapter 386) with the cranial screw, and coupling the adapter to a sheath system.

At 908, process 900 can include a computing system aligning a robotic system with the final trajectory. In some cases, such as when implemented with the robotic system 750, the computing device can register the robotic system 750 with another imaging system such as, for example, the guidance system 536, which can include placing an optical marker (e.g., the optical marker 560) or other imaging fiducial on a portion of the robotic system 750 (e.g., the housing 760). In this way, the coordinate systems of the imaging system and the robotic system 750 can be related and utilized together by the computing device. In some non-limiting examples, the computing device via the guidance system (or other imaging system) can utilize a current (or previous) placement of an optical marker or other imaging fiducial placed on the head of the subject (e.g., to relate the position of the head of the subject with the robotic system 750). In some cases, the robotic system 750 can be mounted on a table that the subject is positioned on. For example, the rail 754 of the robotic system 750 can be secured relative to the subject accordingly. Then, if the robotic system 750 is situated too far from the target location on the subject's head (e.g., defined previously and also indicated by the cranial screw), the robotic assembly 752 can be moved closer to the target location by translating the robotic assembly 752 along the rail 754 and towards the patient. Then, once the position of the robotic assembly 752 relative to the subject (which can have their head fixed, by a head fixation device) is at the desired location, a computing device can cause this position to lock (e.g., by activating a brake).

In some non-limiting examples, once the position of the robotic assembly 752 relative to the subject is at the desired location, a medical instrument to be deployed can be interfaced with the robotic assembly 752. For example, the medical instrument can be inserted through a shaft of the robotic assembly 752 (e.g., the shaft 766) until the medical instrument extends out of the housing a particular amount (e.g., extending out of the fixed head 769 a particular amount). A proximal end of the medical instrument can be engaged with a engager of an advancer (e.g., the engager 794 of the advancer 770). In some non-limiting examples, once the medical instrument has been interfaced with the robotic assembly 752, a computing device can cause the motors (e.g., the motors 764, 772) to collectively align the distal end of the medical instrument along the final trajectory (e.g., the 3D line or an extension of the 3D line that extends along the 3D line).

In some cases, such as when implemented with the robotic arm 850, the computing device can register the robotic arm 850 with another imaging system, such as, for example, the guidance system 536, which can include placing an optical marker (e.g., the optical marker 560) or other imaging fiducial on a portion of the robotic arm 850 (e.g., the end effector 876). In this way, the coordinate systems of the imaging system and the robot arm 850 can be related and utilized together by the computing device. In some non-limiting examples, the computing device via the guidance system (or other imaging system) can utilize a current (or previous) placement of an optical marker or other imaging fiducial placed on the head of the subject (e.g., to relate the position of the head of the subject with the robot arm 850). In some non-limiting examples, such as when the computing device has registered the coordinate system of the robot arm 850 to the other appropriate systems, the computing device can cause the robot arm 850 to align its axial axis that is defined at the end effector of the robot arm 850 and which defines a movement direction of the medical instrument interfaced with the end effector along the along the final trajectory (e.g., the 3D line or an extension of the 3D line that extends along the 3D line). In some cases, a medical instrument can be interfaced with the robot arm 850 before or after aligning the axial axis of the robot arm 850 with the final trajectory, which at least partially extends along the axial axis of the robot arm 850.

At 910, process 900 can include a computing device inserting and advancing a medical instrument that is interfaced with the robotic system. For example, the computing device can activate a motor to cause the medical instrument to advance along the final trajectory. As a more specific example, such as when implemented with the robotic system 750, the computing device can cause the motor to maintain tension on the wire of the medical instrument while causing another motor (e.g., of the advancer) to advance the medical instrument along the 3D line (e.g., the final trajectory) until reaching the target location within the ventricle of the subject. As another specific example, such as when implemented with the robot arm 850, the robotic arm 850 can activate a motor to cause the medical device to advance along the 3D line (e.g., the final trajectory) until reaching the target location within the ventricle of the subject. In some cases, the computing device can, based on the length of the final trajectory (e.g., the 3D line), determine the required extension length for the medical instrument. Thus, the computing device can cause the motor to advance the medical device until the motor extends the medical device the required extension length, and accordingly stop the extension of the medical device when reaching this required extension length (e.g., by stopping the motor). In some non-limiting examples, the final position of the medical instrument can be verified by imaging the subject.

At 912, process 900 can include a computing device draining CSF fluid via the medical instrument, such as when the medial instrument is implemented as a catheter. For example, the computing device can cause a pump that is in fluid communication with a port (e.g., the side port 384 of the sheath system 380) to pump CSF fluid from the ventricle, through the medical instrument (e.g., the catheter), out the port, and into a reservoir (or other collection device).

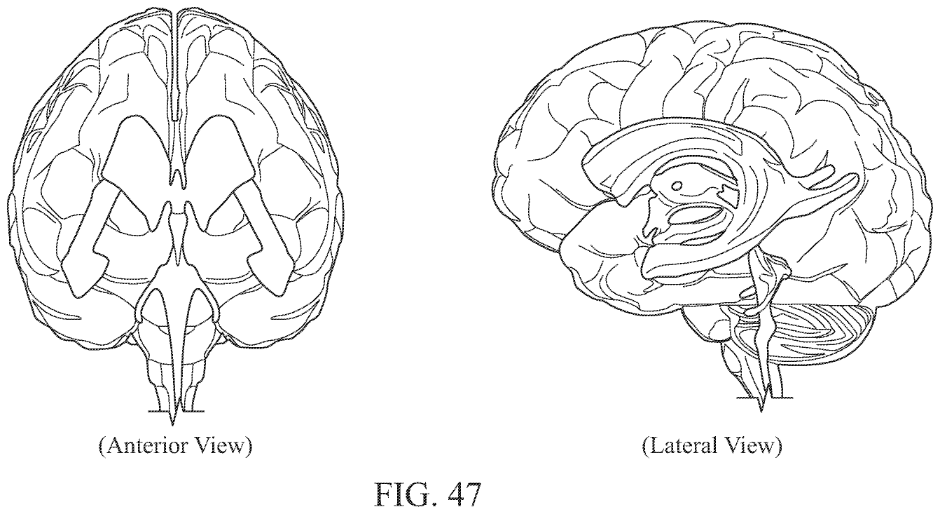

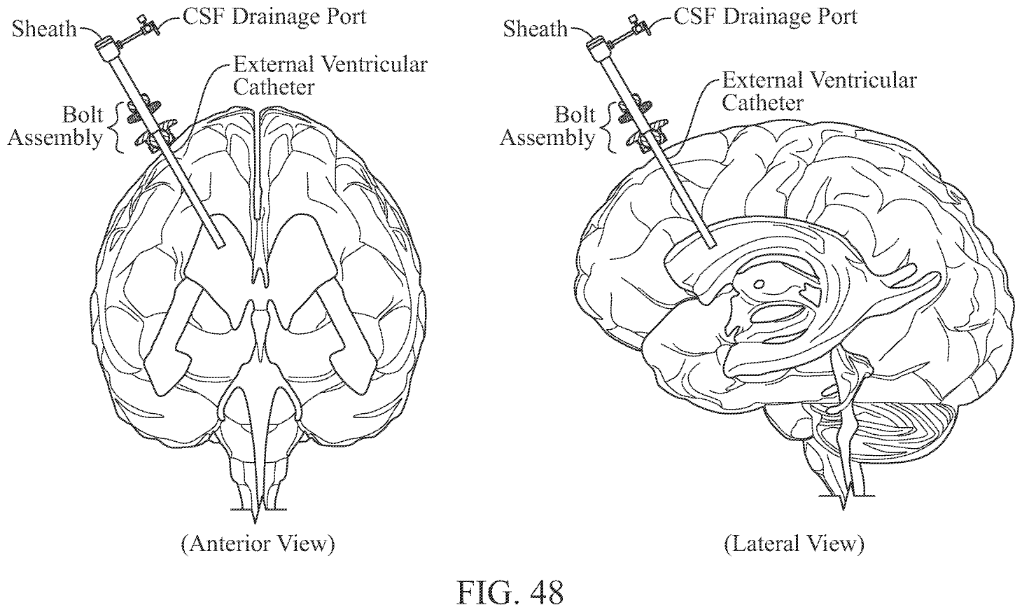

FIG. 47 shows an illustration of an anterior view and an illustration of lateral view of a brain of a subject prior to deployment of a surgical systems. FIG. 48 shows an illustration of an expandable catheter that is an external ventricular catheter, inserted through the top of the sheath, through an adapter, through the cranial bolt assembly, through the subjects brain and into the frontal horn of the ventricle. As shown, this catheter extends along the final trajectory (e.g., the 3D line).

Referring back to FIG. 46, at 914, process 900 can include a computing device expanding the medical instrument. In some cases, the medical instrument has been deployed with a coaxial sheath disposed around the medical instrument to maintain the medical instrument in a compressed state. In some cases, once the medical instrument is in the desired position, the coaxial sheath can be installed with the advancer (e.g., the coaxial sheath engaging with the advancer 770). In this way, the motor of the respective robotic system can be activated in an opposite direction to retreat the coaxial sheath away from the subject. As the coaxial sheath is removed from engagement with the medical instrument, the medical instrument expands radially to a final expanded configuration. Thus, the medical instrument in the expanded state can provide an internal volume that can be defined as a surgical corridor to easily and quickly receive other medical instruments (e.g., surgical instruments) for treating a disease state of a ventricle, such as a hemorrhage. In some non-limiting examples, the practitioner can remove the coaxial sheath or remove another restraining device of the medical instrument to expand the medical instrument to the final expanded state.

In some non-limiting examples, the computing device can include a radial actuator that can be inserted, through the expandable catheter to mechanically contact the radial walls of the expandable catheter to radially expand the expandable catheter. In some non-limiting examples, the radial actuator is hydraulically actuated, electrically actuated, etc. The radial actuator can have an axial hole that is formed after expansion of the expandable catheter (or other expandable instrument) so that other medical instruments can be received through the expanded catheter. In some cases, once the procedure is completed a computing device can cause the radial actuator to retract thereby causing the expandable catheter to retract. In this way, once the procedure is done the radial actuator can be removed, and the expandable catheter can be retracted to a smaller diameter to be retreated out of the subject.

In some non-limiting examples, a cooling or heating device (controllable by the computing device) can be in (or brought into) thermal communication with the expandable catheter or surgical scaffold. In this way, the computing device can cause the cooling device or heating device to maintain a desired confirmation of the expandable catheter or surgical scaffold, such as the compressed (or retracted configuration) and the radially expanded configuration.

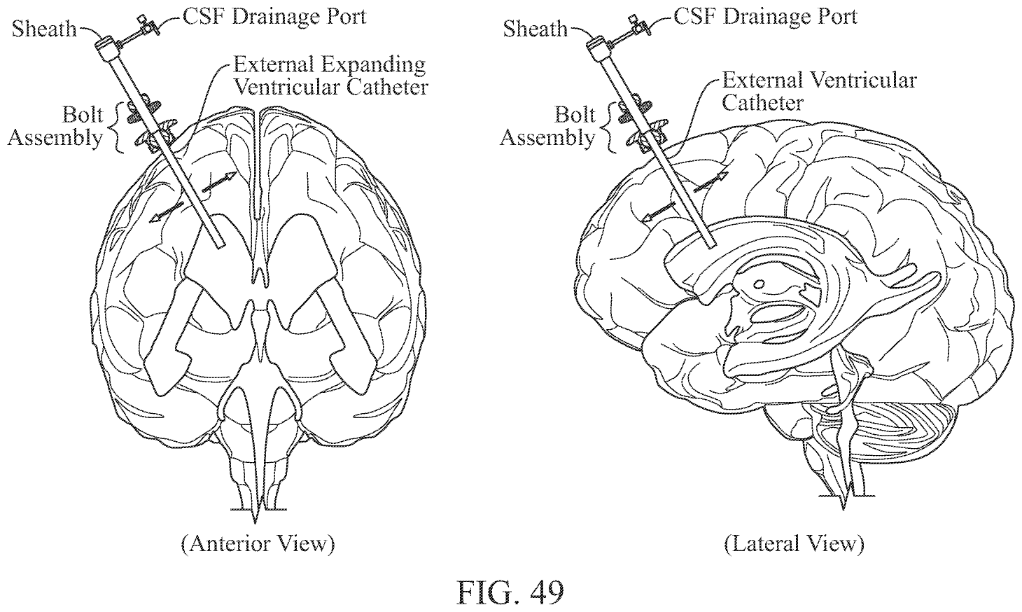

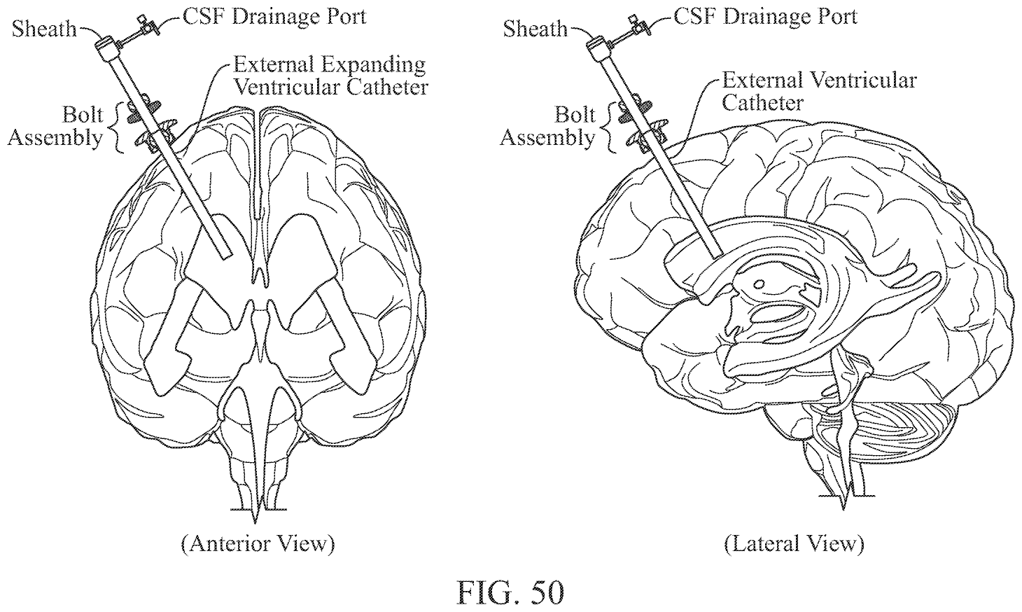

FIG. 49 shows an illustration of the expandable catheter of FIG. 48 prior to expansion, in the compressed state. FIG. 50 shows an illustration of the expandable catheter after expansion, in the expanded state. As shown, the expandable catheter expands radially about the final trajectory (e.g., the 3D line).

Referring back to FIG. 46, at 916, process 900 can include a computing device advancing another medical instrument through the expanded medical instrument to another target location. In some cases, the expanded medical instrument can be removed from engagement with the respective robotic system. Then, another medical instrument can be installed with the respective robotic system (as described above), and the computing device can cause the another medical instrument to advance through the expanded medical instrument. In other cases, the practitioner can advance another medical instrument through the expanded medical instrument. In some non-limiting examples, the medical instrument can be another expandable catheter, a surgical scaffold, etc.

In some non-limiting examples, the another medical instrument can be a surgical scaffold in a compressed (or collapsed state), such as constrained by a coaxial sheath, which is smaller in diameter in the collapsed state as the diameter of the expandable medical instrument in the expanded state. In this case, for example, the surgical scaffold can be engaged with the robot system (e.g., the robotic system 750), aligned and advanced within the expanded medical instrument using a similar procedure above. Once the surgical scaffold reaches the end of the expanded medical instrument, the orientation of the surgical scaffold can be changed to reach a particular target region within the ventricle. In particular, the computing device can adjust the tension on the wire of the surgical scaffold thereby adjusting the orientation of the distal end of the surgical scaffold so that the surgical scaffold can extend along non-linear paths that deviate from the final trajectory (e.g., the 3D line). Thus, the computing device by determining the target location within the ventricle and how far the surgical scaffold has extended, can adjust the tension of the wire, and cause the advancer to advance the surgical scaffold, until the distal end of the surgical scaffold has reached its target location within the ventricle. Once the surgical scaffold has reached its desired location, the surgical scaffold can be expanded from the compressed state to the expanded state (e.g., by the computing device causing a coaxial sheath surrounding the surgical scaffold to be removed and retreated away from the subject). In some cases, the diameter of the expanded surgical scaffold can be slightly larger (e.g., 5% larger) than the diameter of the expandable catheter. In this way, the radial expansion of the surgical scaffold causes the surgical scaffold to abut against the expanded catheter, creating a relatively fixed interface between the surgical scaffold and the expandable catheter.

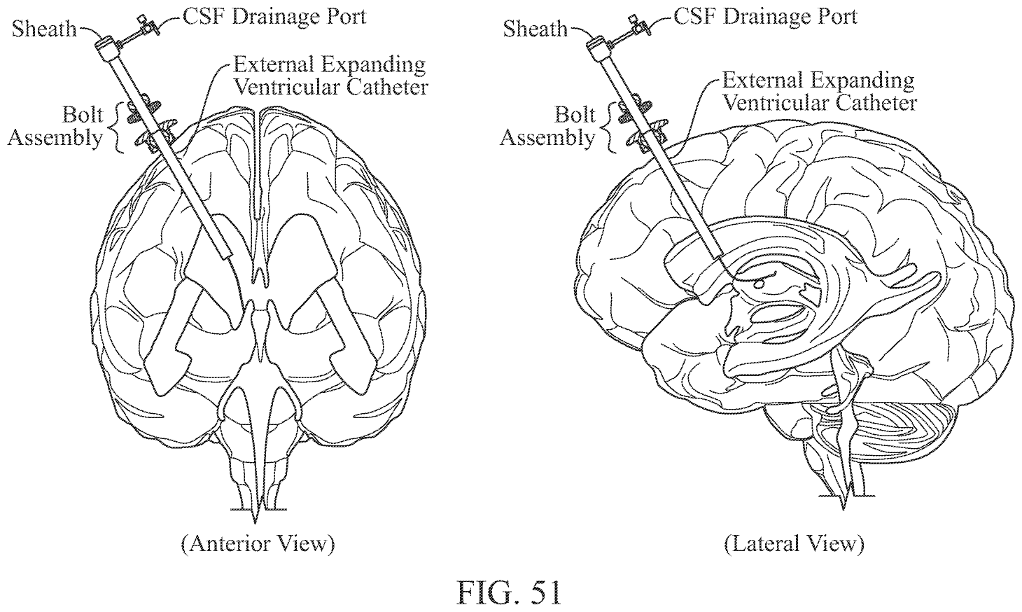

FIG. 51 shows an illustration of the expandable catheter of FIGS. 48-50 with a surgical scaffold that is positioned within the expandable catheter. As shown, a distal end of the surgical scaffold extends out from the distal end of the expandable catheter along a non-linear path. In some non-limiting examples, and as described above, the surgical scaffold (acting as a guidewire in the un-deployed or compressed state as the compressed configuration is compact, firm, and steerable), is computer guided to a desired location within the ventricle via a robotic system (e.g., via “flexible sterotaxy”). In some cases, the robotic system, using computer guidance, navigates the surgical scaffold (e.g., configured as a “guidewire”) to the ideal location on the ventricular surface, which gives access to the medial aspect of the surgical target located at or near the ventricular surface in the brain parenchyma. This navigation is done in a non-linear trajectory following the space provided for by the walls of the ventricle (or an area in the sub-arachnoid space in a different instance). This computer guided, robotically delivered, non-linear trajectory is follows the previously termed “flexible stereotaxy”. Once in place the surgical scaffold (e.g., the “guidewire”) can expand into its expanded form.

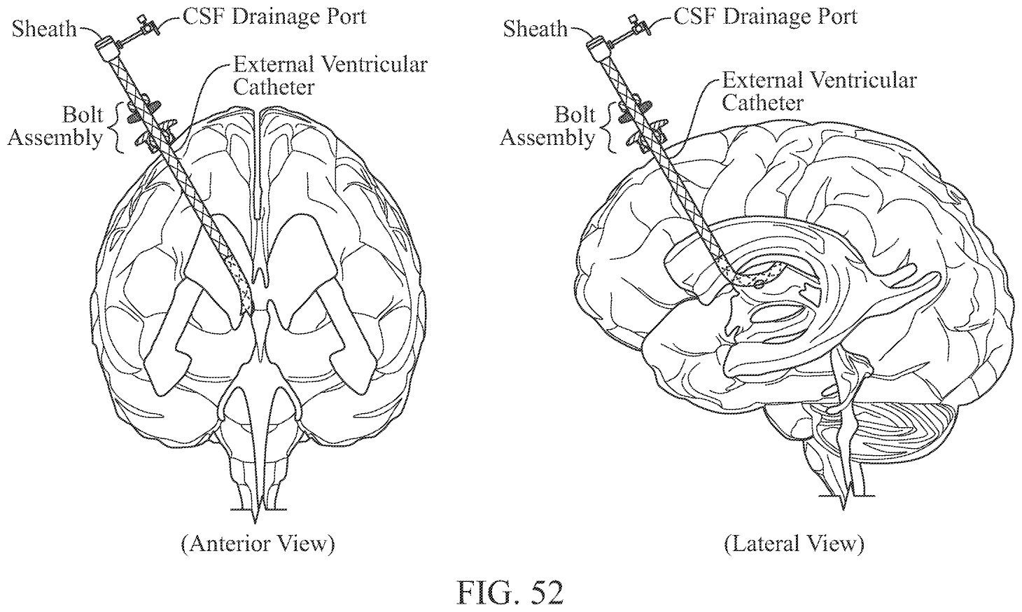

FIG. 52 shows the surgical scaffold situated within the expandable catheter of FIGS. 48-51 in an expanded configuration. The surgical scaffold was previously maneuvered via a robotic system along a trajectory that was computer planned and robotically deployed (e.g., instituted through “flexible stereotaxy”). Once in place the surgical scaffold is expanded, it becomes rigid, which can allow for the subsequent instruments to be passed therethrough without risk of injury to any of the brain tissue outside of the deployed stent. This “structured surgical corridor” that includes the expandable catheter and the surgical scaffold now extends from the surface of the brain, though a non-linear trajectory utilizing the previously placed ventricular catheter and the space within the ventricles themselves, to reach a surgical target on or near the surface of the ventricle.

Referring back to FIG. 46, at 916 process 900 can include advancing yet another medical instrument through the expandable catheter (and other deployed instruments). For example, once the surgical scaffold and the expandable catheter have been deployed and expanded to define a non-linear trajectory, another medical instrument can be inserted through the surgical scaffold (and the expandable catheter). In some non-limiting examples, the medical instrument is flexible, so as to be maneuvered within the surgical scaffold, and in particular being maneuvered along the non-linear trajectory defined by the surgical scaffold. In some cases, this medical instrument can be an (ultrasonic) aspirator. Once the (ultrasonic) aspirator reaches the particular location (e.g., past the distal end of the surgical scaffold), the computing device can cause the ultrasonic aspirator to draw fluid back up the ultrasonic aspirator.

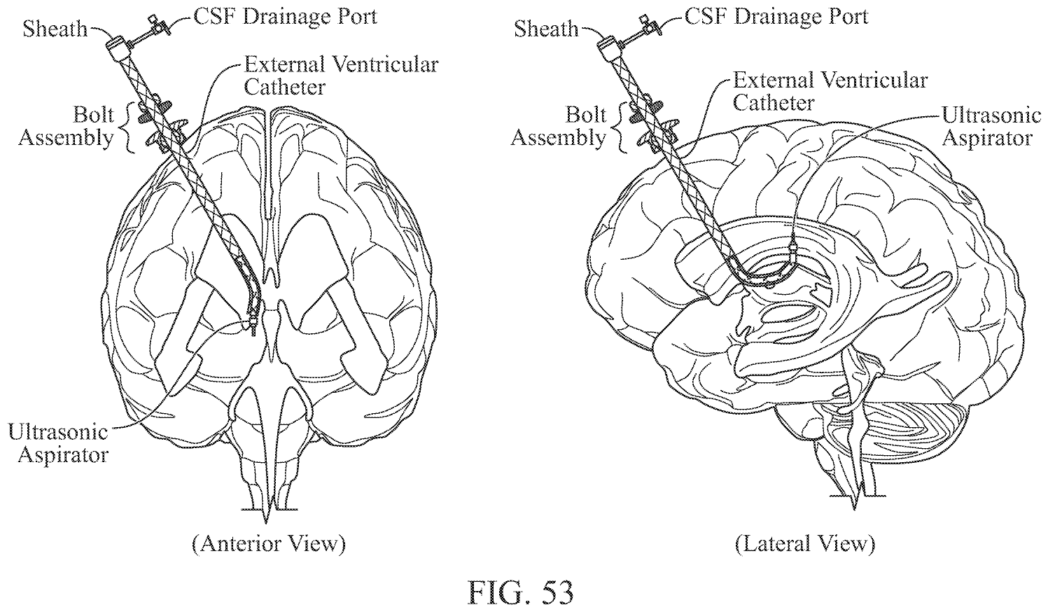

FIG. 53 shows the surgical scaffold situated within the expandable catheter of FIGS. 48-51 in an expanded configuration, with an ultrasonic aspirator deployed through the surgical scaffold and extending beyond the distal end of the surgical scaffold.

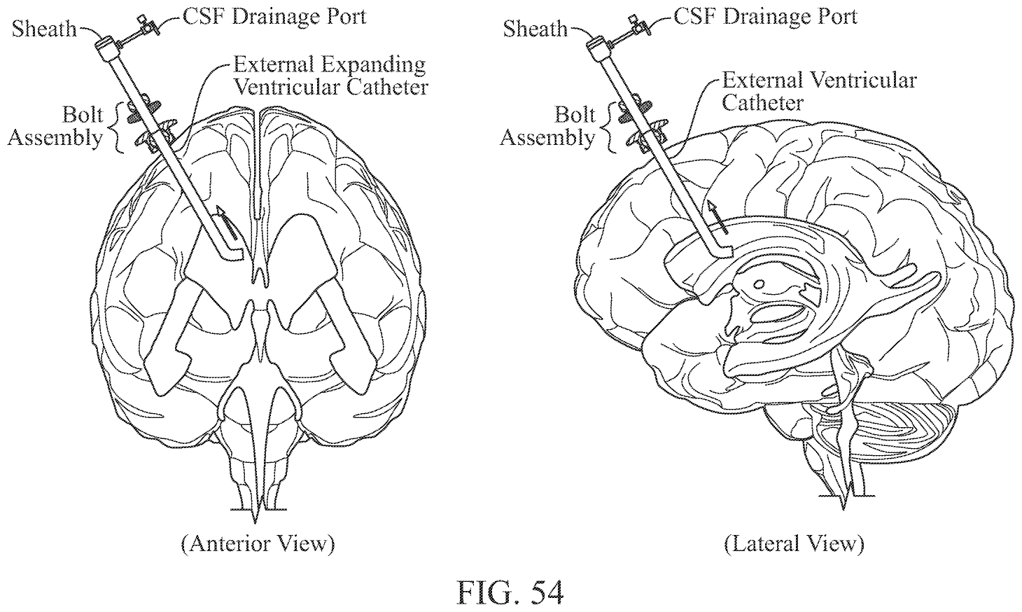

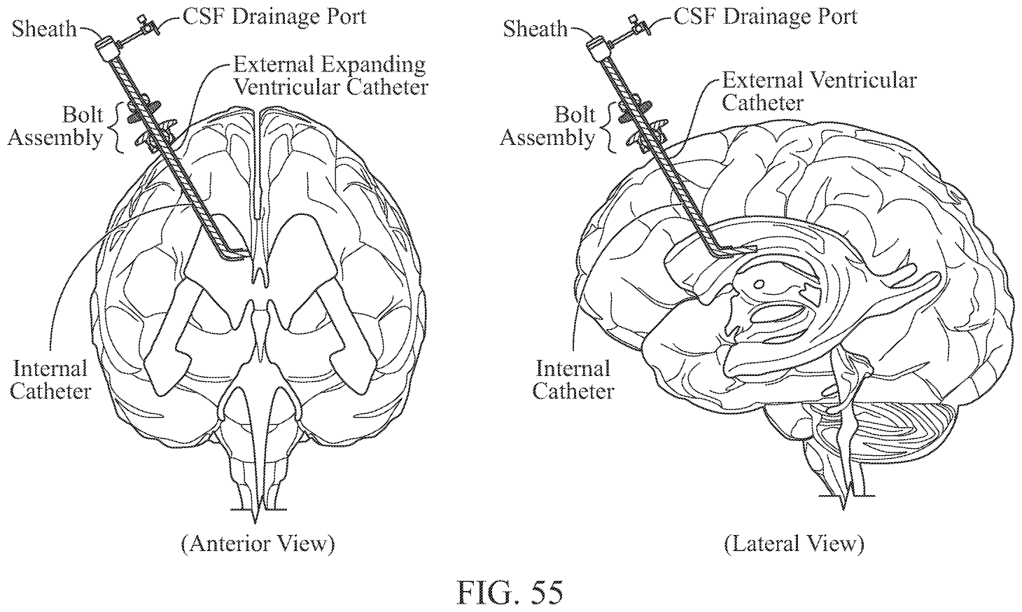

FIG. 54 shows an illustration of another expandable catheter that is also an external ventricular catheter, inserted through the top of the sheath, through an adapter, through the cranial bolt assembly, through the subjects brain and into the frontal horn of the ventricle. As shown, this catheter extends along the final trajectory (e.g., the 3D line). In particular, FIG. 54 demonstrates another example through which “flexible stereotaxy” can be carried out as well as a separate non-limiting example of how to create an “artificial surgical corridor”. In this non-limiting example, an expandable that is a ventricular catheter has at least two unique properties: First is the capacity demonstrated above of the radial or circumferential expansion. In this non-limiting example the expandable catheter will not only have the capacity for circumferential expansion but also has the capacity for guided navigation based on multiple computer/robotic controllable and trackable internal joints. For example, the expandable catheter in the compressed configuration initially has an outer diameter of 3.5 mm, but the outer diameter expands to 8-10 mm in the expanded configuration. This capacity to expand in its dimension allows for the gentle retraction of brain tissue away from the ventricular catheter, which can reduce any damage created by the passage of the originally placed ventricular catheter as it would have the standard dimensions of existing ventricular catheters (e.g., having an average diameter of 3.5 mm). The purpose of this expansion is to allow for the introduction of instruments through the catheter itself for subsequent surgery. This catheter also has the capacity to be steerable though computer guidance−as described above−allowing for the creation of the catheter to follow a non-linear path within the space of the ventricle (see, e.g., the curved distal tip of the expandable catheter of FIG. 54). This capacity allows for “flexible stereotaxy”. This catheter can be driven completely by computer guidance and robotic assistance so that full control of all degrees of freedom will be controlled by the computing device.

In some non-limiting examples, once the robotic guidance has been completed, the robot can be made to become rigid by locking all its joints used for steering of the catheter to the surgical target. Once these joints are locked via the computer control, the catheter will become an “structured surgical corridor” through with (surgical) instruments can be delivered to the target surgical site without risk to the surrounding tissue. These instruments can include other catheters, ultrasonic aspirators, lasers, tissue vaporizers, coagulation devices, hemostatic devices, cutting devices, etc.

FIG. 55 shows an example of a second instrument (which is a catheter) that is deployed through the expandable catheter that is in an expanded configuration. In particular, FIG. 55 shows the catheter extending out of the distal end of the expandable catheter. In some non-limiting examples, the a second instrument can be ultrasonic aspirators, lasers, tissue vaporizers, coagulation devices, hemostatic devices, cutting devices, etc. In some non-limiting examples, it can be appreciated that the components described above be formed out of non-metallic materials, so as to allow for the utilization of CT or MRI usage after installment.

### EXAMPLES

The following examples have been presented in order to further illustrate aspects of the disclosure, and are not meant to limit the scope of the disclosure in any way.

### Part VI: Trial of Software and Device in Phantoms—Cadavers

In this description, we have described the novel computer program to develop automated and ideal ventriculostomy trajectories for placement of the device as well as the 3D-printing of a guidance mask to translate the created trajectory to the patients in real time. In this section we describe our experience reducing these concepts to practice by performing 24 ventricular catheter placement in 6 separate phantoms-cadavers.

### Details of Procedure Testing on Phantom Models

The phantoms utilized are cadaver heads secured under Institutional Review Board (“IRB”) guidance for this purpose. A total of 6 cadaver heads were utilized and 3 placements were performed into the frontal horn of the lateral ventricle on each side. Therefore 6 placements were made per cadaver head therefor totaling 36 total placements.

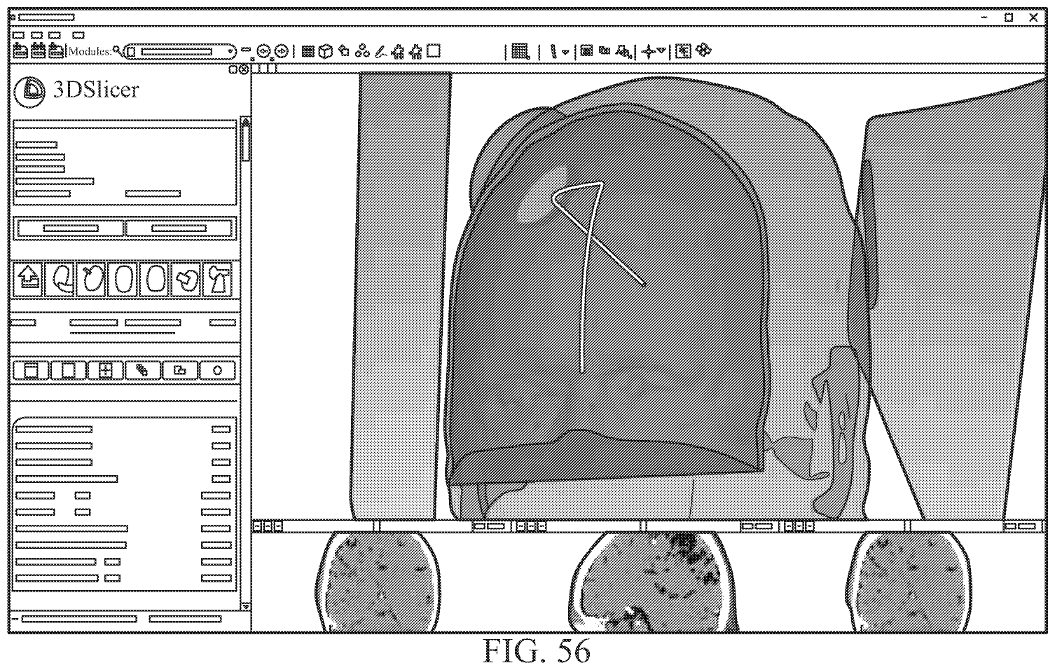

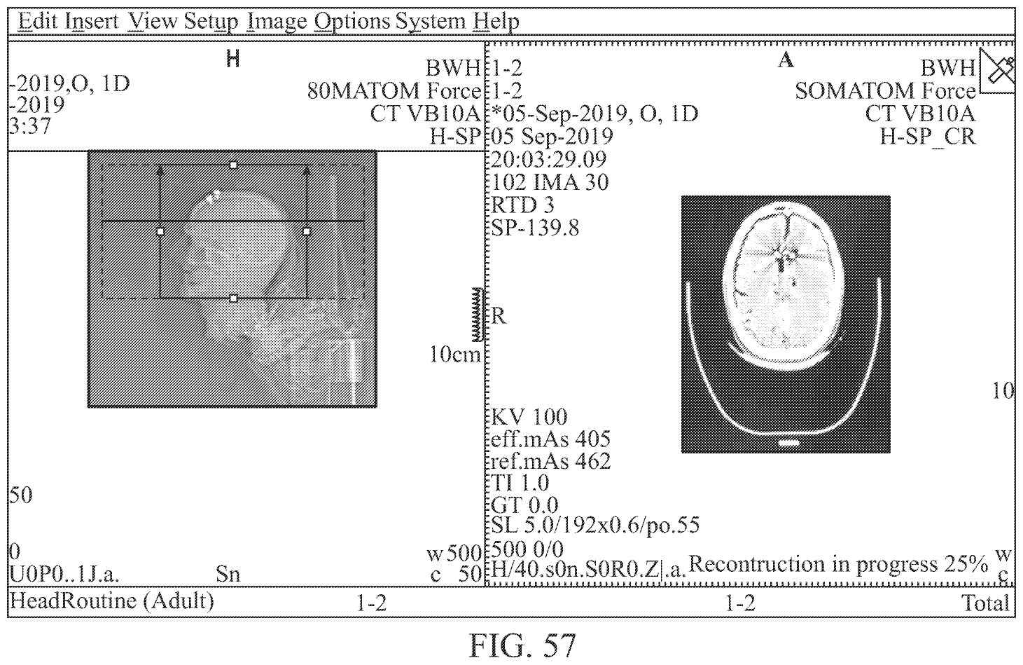

Each cadaver head was placed in a clinical CT scanner at the Brigham & Women's Hospital following IRB overview. Each scan was then processed by the software previously described above (e.g., see FIG. 56). Using the DICOM images and the computer software developed we created an ideal and separate trajectories into the frontal horn of the lateral ventricle of each side with the skull entry point being in the vicinity of Kocher's point (approximately 10 centimeters back from the Nasion in the midline and 3 centimeters lateral to the midline). A 3D printed mask was created for the trajectory and placed on the cadaver head. The procedure performer drilled a hole in the cadaver skull using the guidance of the 3D-printed mask-guide. The skull screw was placed in the bone and the mask and guide system was removed. An EVD catheter and stylet were advanced through the guide, into the hole through the cadaver brain and ventricle until the pre-planned depth is reached. The stylet was removed, and a post-procedure CT was obtained to confirm accurate ventricular placement (e.g., see FIG. 57).

The results of our work demonstrate that we had accurate placement of the ventricular catheter into the selected site in the ventricle through the pre-created trajectory in all of the cadaver experiments performed. The accuracy was measured by calculating the distance between the distal point of the catheter in the ventricle and the ideal trajectory terminus, which averaged 2.2 mm over 36 procedures. This demonstrates greater accuracy than any other reported guidance system to date.

The workflow was described above but will be repeated here. Immediately after the patient completes CT imaging, the software will calculate the recommended entry site, trajectory angle, and catheter depth for the patient and the 3D model will be digitally constructed and then printed in real time. The 3D printed frame will then be transported to the patient's bedside. The procedure performer (neurosurgeon/neurosurgical trainee) will complete the initial skin prep prior to placing the printed guide over the patient's eyes and fastening an adjustable elastic strap to secure placement. The skin is re-prepped through the opening in the mask, and the patient is covered in sterile drapes. A sterile mask platform dimensioned according to the final trajectory is fastened to the mask. A scalpel incision is made in the skin at the trajectory-driven entry site. Then, the bore (or the port) is used to guide a drill to create the cranial burr hole. Once sufficiently drilled, the cranial screw is placed in the cranial burr hole, and the hollow core will serve as the entry passage for the EVD system. After the cranial screw has been implanted, the custom mask can be removed and other components of the securement system can be installed. To ensure that the catheter maintains the intended trajectory angle and depth, a guide wire housed within the catheter during placement provides rigidity. To ensure the catheter is inserted to the correct depth, a stopper device intersects with the bolt to prevent users from too shallow or too deep of an insertion.

The present disclosure has described one or more preferred non-limiting examples, and it should be appreciated that many equivalents, alternatives, variations, and modifications, aside from those expressly stated, are possible and within the scope of the disclosure.

It is to be understood that the disclosure is not limited in its application to the details of construction and the arrangement of components set forth in the following description or illustrated in the following drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

1. A trajectory determination system for guiding a medical instrument, the system comprising:
a computing device, the computing device having a first processor configured to:
receive three-dimensional imaging data acquired by an imaging system, the three-dimensional imaging data being from a head of a subject;
determine vascular structures from the three-dimensional imaging data;
generate a three-dimensional model of the head of the subject from the three-dimensional imaging data, the three-dimensional model of the head including a three-dimensional model of the vascular structures and a three-dimensional model of a portion of a frontal horn of the subject;
determine an entry point on the three-dimensional model of the head of the subject;
determine a plurality of trajectories for the medical instrument, wherein each trajectory within the plurality of trajectories intersects the three-dimensional model of the portion of the frontal horn and does not intersect the three-dimensional model of the vascular structures, and wherein each trajectory within the plurality of trajectories is linear; and
select a final trajectory for the medical instrument from the plurality of trajectories.

2. The trajectory determination system of claim 1, wherein the three-dimensional imaging data includes a plurality of images of the head of the subject, and
wherein a machine learning model identifies the vascular structures from the plurality of images; and
wherein the first processor is configured to generate the three-dimensional model of the vascular structures from the identification of the vascular structures by the machine learning model.

3. The trajectory determination system of claim 2, wherein the first processor is further configured to:
refine the plurality of trajectories to determine a final trajectory being a single trajectory within the plurality of trajectories.

4. The trajectory determination system of claim 3, wherein the first processor is further configured to:
determine for each given trajectory, the closest distance between the given trajectory and the three-dimensional model of the vascular structures; and
determine the final trajectory by identifying the trajectory with the smallest closest distance between the given trajectory and the three-dimensional model of the vascular structures.

5-12. (canceled)

13. The trajectory determination system of claim 1, further comprising:
an external ventricular device including a catheter, the catheter being configured to be received through a hole of a cranial screw.

14. The trajectory determination system of claim 13, further comprising:
a surgical system including a guidewire, the guidewire being configured to be received through the catheter of the external ventricular device.

15. The trajectory determination system of claim 1, further comprising:
a guidance system comprising:
a second processor in communication with the first processor;
a first camera and a second camera, the first camera and the second camera being in communication with the second processor;
a projector in communication with the second processor;
a mounting unit coupled to the first camera and the second camera; and
wherein the second processor is configured to:
receive the final trajectory from the computing device;
cause the first camera to acquire a first image of the head of the subject, and the second camera to acquire a second image of the head of the subject;
construct a three-dimensional surface map of the head of the subject from the first image and the second image; and
register the three-dimensional surface map relative to the three-dimensional model of the head of the subject to track the guidance system relative to the subject.

16. The trajectory determination system of claim 15, wherein the projector is coupled to the mounting unit, and wherein the second processor is further configured to:
cause the first camera to acquire a third image of the head of the subject and the second camera to acquire a fourth image of the head of the subject when the subject has been draped and an optical marker has been placed on the head of the subject, the third image and the fourth image containing the optical marker;
track the position of the guidance system relative to the subject based on the location of the optical marker within the third and fourth images.

17. The trajectory determination system of claim 16, wherein the second processor is further configured to:
cause the projector to emit an illumination pattern towards the subject's head, the illumination pattern aligning with the final trajectory.

18. The trajectory determination system of claim 17, wherein the processor is further configured to:
adjust an angle the illumination pattern is emitted from the guidance system, such that the illumination pattern remains aligned with the final trajectory.

19. The trajectory determination system of claim 16, further comprising:
an augmented reality headset in communication with the second processor of the guidance system, the augmented reality headset being configured to:
project a 3D image scene to a user wearing the augmented reality headset, the 3D image scene including the three-dimensional model of the head of the subject and the final trajectory displayed relative to the three-dimensional model of the head;
track the position of the augmented reality headset relative to the guidance system; and
adjust the projection of the 3D image scene to the user, based on tracked position of the augmented reality headset relative to the guidance system.

20. The trajectory determination system of claim 18, further comprising:
a pivotable screw, the pivotable screw having a main body and a pivotable member received within the main body, the pivotable member being configured to adjust an orientation of the pivotable member relative to the main body, and
wherein the pivotable screw is configured to receive a catheter of an external ventricular device.

21. The trajectory determination system of claim 20, wherein the pivotable screw includes a locking member, the locking member being configured to fix the orientation of the pivotable member relative to the main body.

22. The trajectory determination system of claim 1, wherein the first processor of the computing device is further configured to:
display the final trajectory and the three-dimensional model of the head of the subject on a display in communication with the computing device.

23-39. (canceled)

40. A computer-implemented method for conducting a neurosurgical procedure, the method comprising:
receiving, using one or more computing devices, a final trajectory that is a 3D line;
projecting, using the one or more computing device, an illumination pattern that extends along the 3D line;
aligning, using the one or more computing devices, a portion of an medical instrument so that the portion of the medical instrument aligns with the 3D line; and
advancing, using the one or more computing devices, the medical instrument to extend farther along the 3D line and into a ventricle of a subject.
